SEQUENCE LISTING (44 sequences)

SEQ ID NO: 1 - DNA, 5437 bp, Homo sapiens
  ref <308>: DATABASE ACCESSION NUMBER: GenBank Accession No.: NM_002522
agtgggggcctgatagcgcggcggtgtggaccgcgcggccgaagagcgcggcgcccagagcgcgggccgctcgcggagccacagcccgagccgggtcccagccggagccgagccccagccgagccgagccgggcccggagcgcccggtgcccgcagccatgccggccggccgcgccgcgcgcacctgtgcgctgctcgccctctgcctcctgggcgccggggcccaggatttcgggccgacgcgcttcatctgcacctcggtgcccgtggacgccgacatgtgcgccgcgtccgtggccgccggcggcgccgaggagctccggagcagcgtgctgcagctccgcgagacggtgctgcagcagaaggagaccatcctgagccagaaggagaccatccgcgagctgaccgccaagctgggccgctgcgagagccagagcacgctggaccccggagccggcgaggcccgggcgggcggcggccgcaagcagcccggctcgggcaagaacaccatgggcgacctgtcccggacaccggccgccgagacgctcagccaactcgggcaaactttgcaatcgctcaaaacccgcctggagaacctcgagcagtacagccgcctcaattcctccagccagaccaacagcctcaaggatctgctgcagagcaagatcgatgagctggagaggcaggtgctgtcccgggtgaacaccctggaggagggcaaggggggccccaggaacgacaccgaggagagggtcaagatcgagaccgccctgacctccctgcaccagcggatcagcgagctcgagaaaggtcagaaagacaaccgccctggagacaagttccagctcacattcccactgcggaccaactatatgtatgccaaggtgaagaagagcctgccagagatgtacgccttcactgtctgcatgtggctcaagtccagcgccacgccaggtgtgggcacgcccttctcctacgctgtgcccggccaggccaacgagctggtcctcattgagtggggcaacaaccccatggagatcctcatcaatgacaaggtggccaagttgccttttgtcatcaatgatggcaagtggcaccacatctgtgtcacctggaccacccgggacggggtctgggaggcctaccaggatggcacgcagggtggcagtggcgagaacttggcgccctatcaccccatcaagccccagggcgtgctggtgctgggccaggagcaggacactctgggtggtgggtttgatgccacccaggcatttgtgggtgagctggcccacttcaacatctgggaccgcaagctgacccccggggaggtgtacaacctggccacctgcagcaccaaggctctgtccggcaatgtcatcgcctgggctgaatcccacatcgagatctacggaggggccaccaagtggaccttcgaggcctgtcgccagatcaactgagcacggcaggccaggctgagcccgcccgccctcgccccctgcttgtgcggcgatgatctgttttgtgcgtctcttctctcccttttccccaggaatgaaccgaggccgtcgcccctgcacacgcacacgcacacagcctggttttgtcctcatgcacacgaagcagcccctgctcccatctgtccctgaggaagccccacttctctgtaggagcccggactctctcaggcatgccccattcacagctgaagtgggtgctgcaacgtcttgaacaaggcagaagttggtgagaggatctgtgtgtgcgtgtctacatgtgtgtgtctacgtgtgtgcgtgcgtggctgggggaggccttttctttgaggacgtacctcatttccttctttcttctggctttggaaaaatctcatgatgaaaattcatatttgccaactttgttagctgcgtgcgtgctttggggttggtgcaacctcagtacacgcatttgtctttgtttgcaaacctttctcagagcgacatatctttatattgatgtaataaatgtcttttagtggtttgtcaaaggccgggggcgggggctctctacagagaatttttattttgtaatagaagtgaactgtctctgaagggtgaaggcaggccgtcctgggatggtaccctgtgctctcccgtggaggagaggggatggctgaggacactggcccttaccccagggccagacagcatccatccctgctgtttgcatctgagagcagcatggggcctgggaggtcggcctgtgtgcccagctcagctagctctgccccaggacggccctgccctcgaccttcccacctcctcagatcctgcaaggctggggtctgcccctcccttctcacctctggagctgtgctgcactgcttcagcccagagggccctgagagaggagcgtgccacccacagcccgggaagccgggccccagcacccctctcctttggcctccggcagtgcagaccagaggggaccttttaaggaaagaagccgtgtttcgatgaagacctggccacatggggccactgggacttcaacccagcccatcggtgggaaggtcctttttgggggactttgacagccatatccctcccagcacaccaggcgccaggtgagctggttcagacccctccaggggtactccagagacctcacgtgtggagccaggcctggccagggcaggggcctgaaacccactcctccatctcatggggctcacggcctacagcagcccacaagctgccactggccggcgacactgacacctgagcagtgtccagaacctttttgcctttttttgttccccgtgaaaagcaacatggacatttccttctagtccttccaaggaggggagagaagtgtatgtgcatttgtgtgtgtgtgtgtgtgttgtgtgtgtgtgtgcgctaagtgagaaagagagcaggctcgggaggccctgcccagggtaggaggagcttcctgctttgcaccatctggtggtcgcacgccctgagggcaccccgactctgtctccaggagtctcatcagcaaaccgctgacaagtctttctagaaattctactgcactgcctggctcagctgcagctgcagacatttctgcaggaggagcaggtgtttctgtcttctgttccttctagggccacctgtccccttaaacacaggtccacgttgtgtcaagaacctagtgcatctgtgtgtgtctgtcagtgtctctgtgtcagtgttctcgtgggtgtctgcacggtacccggccgccgttctgcaatgcatcactcccgcagaggggggtgcagatcaggcgccgtgctgcgcgttgttgttcaacagtggctttttcttagataatcgtgcttcctcagcgcccgtcgggttgtggcatccttggatctgcagggatcttctccgtttgcatgttcctcggggtggcgtgttccttgctccctgggtccgacatgtgttcccgcacctgcatggactgccccggttctgtgttgtgtgccgagtgccgcccagtgttctgtgaccacccgtgtagctactgaaaatggctgggtaagcaagtcaagggtgttggaggaggtcaagagagagctcagtttccctctccccctccccaaacacaccaagaagcatttttaacgtgtaggttgagaacaagcctaaaggattcccacagctgggagccagcaagagagcttggagtcgcctctctagaccagatctagccccaccctcactccagccatctcggagcccttgtgtaggcaacgcccggtgcgggctgtgtggggtgctcccctgccagcacctccggccagccccgcccctgccgatctactggaccgcagaccaccttctgcccccgtgggccaggtgggagctgtccgttcaggaccatgagccatcctctgccctgactagcgaggggcagagcacaccccagtgcttacgcctccacccctgcagcctcctggcccgctcaccttcctcacccctcctctgacccacccatggtgccagggccgaagctgacctttagctccctcctgccccttgctagggtctgagccaagcccctcgactccctcactgtgttgacacttggcactttgctggccccgagaaaggtcgatgacacagccgcaaatctaatccacgtagttcccatttactccttaatctgattgatgttccctcttgcactgaataatacatgcctctctcaggtaagccattttataaaacaagaagataaaaagcactgttgaggcagtgtttgcttttgccgagctggtgtccgacagctccctgggtgtccggggtgggagagctgttgacagaagctctccgggccctcaggggcttagatcccacttgagtcgtaagccttcttgcttttgataacacagtattatttctcttactgtagaagaaaaagtttattaccaaacaagagtatttttatgaaagaaaaggacaaacctataaattaactcaacctatatctcccttgaaaatactttcaggctccaccaaaacgtagaactgaaagcatgtattttggaagaaagagatacattttgtatgctttcttttccttttgtagattcccagtttattttctaagactgcaaagatcactttgtcaccagccctgggacctgagaccaagggggtgtcttgtgggcagtgagggggtgaggagaggctggcatgaggttcagtcattccagtgagctccaaagaggggccacctgttctcaaaagcatgttggggaccaggaggtaaaactggccatttatggtgaacctgtgtcttggagctgacttactaagtggaatgagccgaggatttgaatatcagttctaaccttgatagaagaaccttgggttacatgtggttcacattaagaggatagaatcctttggaatcttatggcaaccaaatgtggcttgacgaagtcgtggtttcatctcttaaacacagtgtgtaaatttattcaactaacgatgggaaatgtattacttctgtacacagtggactgaagtgcaatttgttgaaagggaacaagtcattgaagagaaaaaaaaagcccaatacttagagtcccaattttgtctcatttgccaaaaaaaaaaaaaaaaaaaaagcaaaccccctatggttgatattgttataatgtatatactgtataatatgaaagagaatcgatgtatctcactttttcattatttgctaaccaaagctgtacatttttcatatgatctgcagccttttgggtatcaaatgggtcaaaaccatgggacctgccacctcccatcagcaattctggaaatgcactatttctactggtattcttgctttttttttttttttcattttcttgctgaaatgacatgaattgttgagtttatttttacacagtaaagagtggagaaagact

SEQ ID NO: 2 - protein, 432 aa, Homo sapiens
  ref <308>: DATABASE ACCESSION NUMBER: GenBank Accession No. NP_002513
MPAGRAARTCALLALCLLGAGAQDFGPTRFICTSVPVDADMCAASVAAGGAEELRSSVLQLRETVLQQKETILSQKETIRELTAKLGRCESQSTLDPGAGEARAGGGRKQPGSGKNTMGDLSRTPAAETLSQLGQTLQSLKTRLENLEQYSRLNSSSQTNSLKDLLQSKIDELERQVLSRVNTLEEGKGGPRNDTEERVKIETALTSLHQRISELEKGQKDNRPGDKFQLTFPLRTNYMYAKVKKSLPEMYAFTVCMWLKSSATPGVGTPFSYAVPGQANELVLIEWGNNPMEILINDKVAKLPFVINDGKWHHICVTWTTRDGVWEAYQDGTQGGSGENLAPYHPIKPQGVLVLGQEQDTLGGGFDATQAFVGELAHFNIWDRKLTPGEVYNLATCSTKALSGNVIAWAESHIEIYGGATKWTFEACRQIN

SEQ ID NO: 3 - DNA, 5284 bp, Mus musculus
  ref <308>: DATABASE ACCESSION NUMBER: GenBank Accession No. NM_008730
gctgacgtcggcggcgccgaggcccctcccgcggctgcataaaaggcgcgggctccgcagcgcaggcggcagtgggggccgaggagcgcggccgagaagactgtgcggcgtagaagcgcggtgctagagcgcgggccgcctgcggagcgactgccccagccagcccgatcccggtctcagcccgatccccaccggagccgagcggccccggtgctccaagtgtccggagccatgctggccggccgcgccgcacgcacctgtgcgctgctcgccctctgcctcctgggcagtggggcccaagatttcgggccgacccgcttcatctgcacttcggtgccggtggatgcagacatgtgtgccgcgtccgtggctgcgggcggcgcggaggagcttcggagcaacgtgctgcagctccgcgagaccgtgctgcagcagaaggagaccatcctcagccagaaggagaccatcagggagctgaccaccaagcttggccgctgcgagagccagagcaccctggactcgggtcccggcgaggccaggtcgggaggcggccgcaagcagcctggctcgggcaagaacacgatgggcgacctgtcccggacgccggccgcggagacgctcagccaactcgggcaaactttgcaatctctcaaaacccgactggagaacctcgagcagtacagccgcctcaattcttccagccaaaccaacagcctcaaggatctgctgcagagcaagatcgatgacctggagcggcaggtgctgtctcgggtgaacactctggaggaggggaaagggggccccaagaacgacacagaggaaagggccaagatcgagagcgccttgacctccctacaccaacggatcagcgagctggagaaaggtcagaaagacaatcgccccggagacaagtttcagctgacattcccactgcggaccaactacatgtatgccaaggtgaagaagagcctgccagagatgtatgccttcaccgtgtgcatgtggctcaagtccagtgcggcgcctggagtgggcacacccttctcttatgctgtgcctgggcaggccaacgaactggtcctcatcgagtggggcaacaaccccatggagatactcattaatgacaaggtggccaagctgccgtttgtaatcaacgatggcaaatggcatcacatctgtgtcacctggaccacccgggatggggtctgggaggcctatcaggatggcacccagggtggcaatggagagaacttggcaccctatcaccccatcaaaccacagggcgtgctggtgctgggtcaggaacaggacactctaggtggagggtttgatgccacccaagcatttgtgggtgagctagcccatttcaacatctgggaccgcaagctgacccccggggaggtgtacaacctggccacctgcagcagcaaggctctctctggcaatgtcatcgcctgggctgagtcccagatcgagatctttggtggagccaccaagtggacattcgaggcttgtcgccagatcaactgaaggcttcaggctagggctgagcccccagccccagcgcctgcccaccctgcttgtgcggtgacccatcgtctctcttctcccctccccccaggaatgactcaaggccatggcccctgcacacgcacacgcacacagcctggttgtcctccttgtgcacttgaagcagtcccggctcttctctggcccccaaggacacccccttctctctaggggccccactatatcctgggcatacctagctaaagcaggcagcatcagctttgacaatgcagaatttcgtgagaaagagagagagagagagacagagagagagagacagagagagagagcgcgagtgtgtgtgtagggaggctttcttctagaatgagttagtttgttcccttctttctgtgtgattgttgtaagtttcctgatgggtcggctgcctgtgtgcctcagggctggtgcggcttcctggaccacccatttgtccttgtttgcagacctctttgggagtgatgtaggtctctgttgacataagtcaacaatggtttgtcaaggttgggggggtgctttccacaggaatatttgggggggatgataagtggagtctctgaaattcaaaggctggttgtccagggatggcaccacctaccaatcagccccttcccacgtccccatcaggctgtctgctgtctacatctgagggtggcatgggctgagcccctcagcagctctaccccaggactgctcctcacctcacccttggccttcctgcctccttgtatcttatcaagggacctgttattccctttcagcttccaaagctacttcagcccagtggttctgagaggagagtgcccttccagagtcccaggacacagggccttggcacccacccttcttcctccctctggcaatgagagaccagaggcttcaagtgggggtctcacgtgctcggtctcagcgctagagaataccgagacctgatccacagtccgtgggaatggtctcccacggcatttttcacagccatactccctccccccagcgcacccaggcccgggtgagctagctcagacccctcaggggccaggggctcagaaacctctcatgtggagcctgctggccggggtgggtcctgccatccactcctgcatttcatggggcacaagcccagcagcagccccaacctgccaccggcctgagacactgagcccgtggcttttccttattgaaagtaggcatgagtacccccttctgggccttctgaggagggggacgaaatgcgtgtgtgcacatgtgtctgttgaatgctgaggaggctgtgtccaggcagggtggacccctgcattgttcccccccgtgttgtgggcacacacccagtgacacctggactatgtctccagtagcctgactagcaggaggctggcaagtctttctagaaacctacgcactgcctggctcgtctgcagctgcagacacttaattcctcaacaggagcaggttttccacctgctgttccttccaggggcacctgtctccttccacacaggtccatgttgtgtctgaaactagtgcatccgtgtctgtctgtgtgctgtctgtgtcactgttcttgtgggtgtctgcacagttccctggtcactgctctgtggtgcatcctctctgagggtgcggatcaggctgtgctccacccgttgtccagcagtggctttttcttagataattgtgcttcctcagcgcccgccgggttgtggcatccttggatctgcggggatcttctgtgtttgcatgttcctcagggtggtgtgtcccttgctcccttggtctgacgcgtgttcccttgcctgcatggacttctctggttctgtgtttgtgtgctgaatgccacccagtgttctgtgatcacccatgtagctactgaaaaatggctgggtaagcaagccaggggtgttggggaggtcaggagagagatcaacatgcctctcccctccccctccccagacatagcaagaagcatttctaacatctaggttgagaacaagcctgaatggtacccatggttgggagagagaacttgaacttcaaatcgactttccagaccagatcgagccccacccttacttttagccttctgggagccaccaccaggcgaggcccagagccggctatgcccggtgtactcctgccaacccctgccaccagccccagcccctgccaatgcggaccccagaccacttgcacccagacgggtggggtgggagctgtccgttcaggaccacaagccatcctctgccctggcttagagggcagagcacaccccaatgcttctgcctctgtcacttcatctctccactcctctcccttctcctctcccctgcggcacccacggtgccagggacagacgctgacatctagctcttgccctgccccttactggagtctgtgccaagccctcctctccgtcactgtgttcacacttggcactttgctggccctgagaaaggtaacgacagccgcaaatctaatccgcatatttcccatcgactccgtaatctgattgatgttccctcttgcactgaataatacatgcctctctcaggtaagccattttataaaataagagataaaacgcactgtcgagacagtgtttgcttctgccgatggtgtccgacagcaccctgggcattagggtggtagagctgccacggaggctcccaggccctaggggctgagagctcacttgaattgtaagccttcttgcttttgataacacagtattatttctcttactgtagaagaaaaagtttattaccaaacaagagtatttttatgaaagaataggacaaacctataaattatctcaacctatatctcccttgaaaatactttcaggctccgccaaaacctaggactgaaagcaagtattttggaagaaagagatacatttttgtatgctttcttttccttttgtagacgcccgacttattttctaagactgcaaagatcactttgtcaccagccctgggacctgaggccaaggggttgtcttgtgggcagtgagggggaaggacaggctggcctgaggttcacagtcattccagtgagctctgatgaggggccaccagatcttgagggtgtgtaagggaccaggaggttaaaaagaaaggcccctgatggtgggctctttgtgttggagctgaaaggttatatggaaggagataagaatttggataccagttctgagcttgatagaaggaattagggtcactctggctaacataggggtacagcatccttagtaacccgagcgtggctcgaagtggtcatgatttcatccaaaacacaatgtgtaaacttactcagctaacaatgggaaatgtattgcttctgtgcgcagtggacttacgtgcaatttgttcaaaagggaacaagtcattgaggagaaacaaaagcccaatacttagagtcccaattttgtcttatttgccaaaaaaaaagcaaccccccaagagttgatattgttacaatgtacatactgtaaaatatgaaagaatcgatgtatcttactttttcattatttgctaaccaaagctgtacatttttcatacaatccgcagccttttgagtatcaaaggggtaaaccacacccatcagccattctggaagtgcactatttatactggtatccttgctttttttttcccattttcttgctgagatgacatgaattgttaagtttatttttacacagtaaagagtgaagaaagac

SEQ ID NO: 4 - protein, 432 aa, Mus musculus
  ref <308>: DATABASE ACCESSION NUMBER: GenBank Accession No. NP_032756
MLAGRAARTCALLALCLLGSGAQDFGPTRFICTSVPVDADMCAASVAAGGAEELRSNVLQLRETVLQQKETILSQKETIRELTTKLGRCESQSTLDSGPGEARSGGGRKQPGSGKNTMGDLSRTPAAETLSQLGQTLQSLKTRLENLEQYSRLNSSSQTNSLKDLLQSKIDDLERQVLSRVNTLEEGKGGPKNDTEERAKIESALTSLHQRISELEKGQKDNRPGDKFQLTFPLRTNYMYAKVKKSLPEMYAFTVCMWLKSSAAPGVGTPFSYAVPGQANELVLIEWGNNPMEILINDKVAKLPFVINDGKWHHICVTWTTRDGVWEAYQDGTQGGNGENLAPYHPIKPQGVLVLGQEQDTLGGGFDATQAFVGELAHFNIWDRKLTPGEVYNLATCSSKALSGNVIAWAESQIEIFGGATKWTFEACRQIN

SEQ ID NO: 5 - DNA, 2024 bp, Homo sapiens
  ref <308>: DATABASE ACCESSION NUMBER: GenBank Accession No. NM_000567.2
aaggcaagagatctaggacttctagcccctgaactttcagccgaatacatcttttccaaaggagtgaattcaggcccttgtatcactggcagcaggacgtgaccatggagaagctgttgtgtttcttggtcttgaccagcctctctcatgcttttggccagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctgaggcccagctgtgggtcctgaaggtacctcccggttttttacaccgcatgggccccacgtctctgtctctggtacctcccgcttttttacactgcatggttcccacgtctctgtctctgggcctttgttcccctatatgcattgcaggcctgctccaccctcctcagcgcctgagaatggaggtaaagtgtctggtctgggagctcgttaactatgctgggaaacggtccaaaagaatcagaatttgaggtgttttgttttcatttttatttcaagttggacagatcttggagataatttcttacctcacatagatgagaaaactaacacccagaaaggagaaatgatgttataaaaaactcataaggcaagagctgagaaggaagcgctgatcttctatttaattccccacccatgacccccagaaagcaggagggcattgcccacattcacagggctcttcagtctcagaatcaggacactggccaggtgtctggtttgggtccagagtgctcatcatcatgtcatagaactgctgggcccaggtctcctgaaatgggaagcccagcaataccacgcagtccctccactttctcaaagcacactggaaaggccattagaattgccccagcagagcagatctgctttttttccagagcaaaatgaagcactaggtataaatatgttgttactgccaagaacttaaatgactggtttttgtttgcttgcagtgctttcttaattttatggctcttctgggaaactcctccccttttccacacgaaccttgtggggctgtgaattctttcttcatccccgcattcccaatatacccaggccacaagagtggacgtgaaccacagggtgtcctgtcagaggagcccatctcccatctccccagctccctatctggaggatagttggatagttacgtgttcctagcaggaccaactacagtcttcccaaggattgagttatggactttgggagtgagacatcttcttgctgctggatttccaagctgagaggacgtgaacctgggaccaccagtagccatcttgtttgccacatggagagagactgtgaggacagaagccaaactggaagtggaggagccaagggattgacaaacaacagagccttgaccacgtggagtctctgaatcagccttgtctggaaccagatctacacctggactgcccaggtctataagccaataaagcccctgtttacttgaaaaaaaaaa

SEQ ID NO: 6 - protein, 224 aa, Homo sapiens
  ref <308>: DATABASE ACCESSION NUMBER: GenBank. Accession No. CAA39671.1
MEKLLCFLVLTSLSHAFGQTDMSRKAFVFPKESDTSYVSLKAPLTKPLKAFTVCLHFYTELSSTRGTVFSRMPPRDKTMRFFIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 7 - DNA, 2738 bp, Homo sapiens
  ref <308>: DATABASE ACCESSION NUMBER: GenBank. Accession No. NM_002523.2
agagtgccgagcagcgcggtgggtgcggctgtgagacggcaggagacttctgccccgcggtgcacgcgaccctcgagacgacagcgcggctactgccagcagcgaaggcgcctcccgcggagcgccccgacggcgcccgctcgcccatgccgagctgagcgcggcagcggcggcgggatgctggcgctgctggccgccagcgtggcgctcgccgtggccgctggggcccaggacagcccggcgcccggtagccgcttcgtgtgcacggcactgcccccagaggcggtgcacgccggctgcccgctgcccgcgatgcccatgcagggcggcgcgcagagtcccgaggaggagctgagggccgcggtgctgcagctgcgcgagaccgtcgtgcagcagaaggagacgctgggcgcgcagcgcgaggccatccgcgagctcacgggcaagctagcgcgctgcgaggggctggcgggcggcaaggcgcgcggcgcgggggccacgggcaaggacactatgggcgacctgccgcgggaccccggccacgtcgtggagcagctcagccgctcgctgcagaccctcaaggaccgcctggagagcctcgagcaccagctcagagcaaacgtgtccaatgctgggctgcccggcgacttccgcgaggtgctccagcagcggctgggggagctggagaggcagcttctgcgcaaggtggcagagctggaggacgagaagtccctgctgcacaatgagacctcggctcaccggcagaagaccgagagcaccctgaacgcgctgctgcagagggtcaccgagctggagcgaggcaatagcgcctttaagtcaccagatgcgttcaaggtgtccctcccactccgcacaaactacctatacggcaagatcaagaagacgctgcctgagctgtacgccttcaccatctgcctgtggctgcggtccagcgcctcaccaggcattggcacccccttctcctatgcggtgccagggcaggccaacgagatcgtgctgatcgagtggggcaacaaccccatcgagctgctcatcaacgacaaggttgcgcagctgcccctgtttgtcagtgacggcaagtggcaccacatctgtgtcacctggacgacacgggatggcatgtgggaggcattccaggacggagagaagctgggcactggggagaacctggccccctggcaccccatcaagcccgggggcgtgctgatccttggacaagagcaggacaccgtggggggtaggtttgatgccactcaggcatttgtcggggagctcagccagttcaacatatgggaccgcgtccttcgcgcacaagaaattgtcaacatcgccaactgctccacaaacatgccgggcaacatcatcccgtgggtggacaataacgtcgatgtgttcggaggggcctccaagtggcccgtggagacgtgtgaggagcgtctccttgacttgtagccgccttctcctctgtccaggaggccgggatcaggctgttgccatggaagttcagggccatagactgccccacttaaactcttgtcagtctgggctcagggttcccagagctcattccccaggaatctctaagaccagggctggggcagtgtctgtcactggcttgtttgttccctaccaatattctgttgctgtttgaagtagtgccagggtcccctgggaagatgcccccaagacacctgccccaagtgggtggatatctgccttcctgctgcaagtggaggcaggtccagcagcccctcttcagagcccctgtaaatgctatcgcagcctgagtcctgccgccttccagttccttggtgtcccgtgcaccccttctgtctgtcccctttcatggctgtgcagccgtcccgctggagtggccatgtcccttgtgcattgagtgcatccccgctggtgactaagctcgcagcaagcggctaccccccgatctgcaaaagggcctctccctttgtgttctatacattgtgaatcttcccgtctgaagaacgcccagcctgcccagacaaagccccgccttccccaaagcagaggggctgtctgtgtctccagaaaggggacatcgggggggaggggggctcagaaaggagaagggctgtgatctccggtcccttcccccatcatccttccttagactgatgctttgactgaatcatcactagctatggcattaaaaggcctctcttctcatctggtgccaaaggttccgttgcagctttttacaaccatccggtgtggtttggaggatttgttttttttttttcccaacagaaaagaacagccattagaagaaggctcccattttctgatgttccgccccactgtgaagagtgtgctcgttttaaattcatgttgattcttgtaagcactggactgtcttcatcaagtatttccctacagaactcctcaagaaaacagagatcatttggctagagattgtctgagtgactccaagctactcactgtattggacgggagtagtaatttattttaaagataaagtgactaagtggggaaatttataaagctaaatattatatattttatttttcatacatgtttgaagtgcaaatctgtggatattccatttgtaggaccaagtcgacatgcccatcctgacattgtatgctacgagaactcttctgatgatggaatttcgattaaagtgcactgaaagatgaaaaaaaaaaaaaaaaaaaaaaaaa

SEQ ID NO: 8 - protein, 431 aa, Homo sapiens
  ref <308>: DATABASE ACCESSION NUMBER: GenBank. Accession No. NP_002514.1
MLALLAASVALAVAAGAQDSPAPGSRFVCTALPPEAVHAGCPLPAMPMQGGAQSPEEELRAAVLQLRETVVQQKETLGAQREAIRELTGKLARCEGLAGGKARGAGATGKDTMGDLPRDPGHVVEQLSRSLQTLKDRLESLEHQLRANVSNAGLPGDFREVLQQRLGELERQLLRKVAELEDEKSLLHNETSAHRQKTESTLNALLQRVTELERGNSAFKSPDAFKVSLPLRTNYLYGKIKKTLPELYAFTICLWLRSSASPGIGTPFSYAVPGQANEIVLIEWGNNPIELLINDKVAQLPLFVSDGKWHHICVTWTTRDGMWEAFQDGEKLGTGENLAPWHPIKPGGVLILGQEQDTVGGRFDATQAFVGELSQFNIWDRVLRAQEIVNIANCSTNMPGNIIPWVDNNVDVFGGASKWPVETCEERLLDL

SEQ ID NO: 9 - DNA, 5831 bp, Homo sapiens
  ref <308>: DATABASE ACCESSION NUMBER: GenBank. Accession No. NM_014293.3
cggccgcggcgacagctccagctccggctccggctccggctccggctccggctcccgcgcctgccccgctcggcccagcgcgcccgggctccgcgccccgaccccgccgccgcgcctgccgggggcctcgggcgcccccgccgcccgcctcacgctgaagttcctggccgtgctgctggccgcgggcatgctggcgttcctcggtgccgtcatctgcatcatcgccagcgtgcccctggcggccagcccggcgcgggcgctgcccggcggcgccgacaatgcttcggtcgcctcgggcgccgccgcgtccccgggcccgcagcggagcctgagcgcgctgcacggcgcgggcggttcagccgggccccccgcgctgcccggggcacccgcggccagcgcgcacccgctgccgcccgggcccctgttcagccgcttcctgtgcacgccgctggctgctgcctgcccgtcgggggcccagcagggggacgcggcgggcgctgcgccgggcgagcgcgaagagctgctgctgctgcagagcacggccgagcagctgcgccagacggcgctgcagcaggaggcgcgcatccgcgccgaccaggacaccatccgtgagctcaccggcaagctgggccgctgcgagagcggcctgccgcgcggcctccagggcgccgggccccgccgcgacaccatggccgacgggccctgggactcgcctgcgctcattctggagctggaggacgccgtgcgcgccctgcgggaccgcatcgaccgcctggagcaggagcttccagcccgtgtgaacctctcagctgccccagccccagtctctgctgtgcccaccggcctacactccaagatggaccagctggaggggcagctgctggcccaggtgctggcactggagaaggagcgtgtggccctcagccacagcagccgccggcagaggcaggaagtggaaaaggagttggacgtcctgcagggtcgtgtggctgagctggagcacgggtcctcagcctacagtcctccagatgccttcaagatcagcatccccatccgtaacaactacatgtacgcccgcgtgcggaaggctctgcccgagctctacgcattcaccgcctgcatgtggctgcggtccaggtccagcggcaccggccagggcacccccttctcctactcagtgcccgggcaggccaacgagattgtactgctagaggcgggccatgagcccatggagctgctgatcaacgacaaggtggcccagctgcccctgagcctgaaggacaatggctggcaccacatctgcatcgcctggaccacaagggatggcctatggtctgcctaccaggacggggagctgcagggctccggtgagaacctggctgcctggcaccccatcaagcctcatgggatccttatcttgggccaggagcaggataccctgggtggccggtttgatgccacccaggcctttgtcggtgacattgcccagtttaacctgtgggaccacgccctgacaccagcccaggtcctgggcattgccaactgcactgcgccactgctgggcaacgtccttccctgggaagacaagttggtggaggcctttgggggtgcaacaaaggctgccttcgatgtctgcaaggggagggccaaggcatgaggggccacctcatccagggcccctcccttgcctgccactttggggacttgaggggggtcatattccctcctcagcctgcccacgcactggccttccctcctgccccactcctggctgtgcctcccatttcccctcacctgtacccacacctccagaatgccctgccctgcgagtgtgtcccctgtccccacctgagtggggaggagcgtctcaagtgaacagtgggagcctgcccacctggcactgcactggagttgtctcttaccccaccctccctgcccatcaactgtatctgatttcactaattttgacagcacccccagtagggtaggattgtgtatgagggggaccccactatctcagtggtgggggtggccgcccgcccccttgtcccccatgcaacaggcccagtggcttccccttcagggccacaacaggctgtagaaggggatgacgaggacatcagaggttagacttaccctcctccctctttccaccagctgccagtcaagggcagtgggatctcgatggagcctccccccccccccacccatgcctccctcttcctcctctttcctcctctctttgtgtgtagcggtttgaatgttggttccatgcctggcccagccccacctcagtctccaggacattcctttcccagctccagcctggagggaaggggacaaagaccccaggaggccaaagggctgcagtcaccccttgtgctcacccatagtgatggccactggtatagtcatcgctctccctccatgccaaggacaggacttggaccgcttcagcctgggctgggagcagccctaaggtagaggcctcatggcccaggagaccccacctctggcagagccacattacctaccctgtgcatggtcctggggcagcaaggaagaagctcagagggtggggagaagcatgaagcagtgagcagagcactgggtgagagggagaagaccttggttcctagccagccctgctaatgtgctgtgtggccttctgtaagtccctgccctctctgggcctggccttcctcattcgtgagctgaggccctcgctttggtcatttgctctccagattgggtgtgagcttctctgtgattccaggtggatatgtggggaaagctctggtgaccctgggcttcgcaggggtagatcccaggactcggcagtggatgggatgcagccagtcatgggttagggtcagcagagactcagagtccagggcaaggttcaaggcagactaacctcatgcatggattgtaaaaaaccagctccctttggatcaacccagcctggcacccttgcctgtctgagagtgtctcaaagggctgatggcttcctggtccccttgagtcatcaccagcttccccaagagagtgtcagaatcttaagagctgagaggccgggcacggtggctcacgcctgtaatcccagcactttgggaggctgagacaggcagatcacttgaggtcaggagttcgaagtcagcctggccaacgtggtgaaaccccatcttcactaaaaatacaaaacttagctggttaggtggtgcatgcctgtagtcccagctactcgggaggccgaggcagaagaatctcttgaactgaggaggtggaggttgcagtgagccgagatcacgccattgcactccagcctgggcaacagagcaagactccatctcaaaaaaataataataatcttaaagatgagaaaagccaccccatctggcaccacagctgcatcttgcttgtgagaaatggggaagagttcagggaggacacgtgacctgcacaggatcacagagcatggggcagagccaggactagagctcagggcatctgactccctcttcagtgttcttccccctccatgttgcctgcccctgaagacctttgagttcagtctacacctaagcaggtagacatccgcgaggtcagatgctttccaacatgacacctgaacatcttcctttatgcaacacccaaacatcttggcatccccaccccaggaagtgcggggaggaggttatgatccctgggcgcttcggcagaatggagagctgaggtgtccctcccctgctagtcacctaccaggtgtctgagcagctgcatgctccctggctcaagtgggcactgtaccttttgcctgcctttttgttccctatctccactccctgaggccacttagcctgagacatgatgcaagagctgcaggccggggggctcagtgccatggaagctactccaagttgcattgcctcccgcgcccagatcctgctttccatttcgagaacataaatagattgcccagcccctccagtacaatcccactggaagaaaaggcaatggcgggcttcagccagacctgctgagacctaggttgccacggtaacagccaaagacatcaacccaagtgctgggtcaagtgtctcatcatactggcactgttgctggggtgacggcagaattcagaacttcaatttcagtgacgccaagcttgatgtgtttctgttattgttttgaagaaggtagctcttgtggaggacttgggagaaggatggggtcttaggaaggaggtgacagcacttgcatggtcacttgagcccacacacacgctcaaccccaagtcctttatgctttgtcacagtgaagatgagacctctgacgtccaagccttgttcctgtgctgcatcacccactcagccttccaaagggaacaggaacaaatttccccagcaccactgtttgggtcccgcttttcctatcttctgctgcccctgagcacatccaagcagacagggaaagaggagtcagacatggcccagtcacatcctgagctgctcctggctgataaccacgatggagcccgtgtttgtcctgccatctggcactgcactgagtgtggcacaggcaccgtcctgttgatctcacaacacagttctaagttaggacgttcttggctccgttagacaggtgaggaaactggggcacagagaggtgatgtcatctgcctggtgtcaatcagctagcaagtgatggagcccagatttcaaaccaaagggggttacgtccaggggctgagttcccactcacctgtgtagagtgccatctgggcaccattgctccagacgtgttccgacccctttcccagcccacagggcttgaagtgaaggaacagaggcagggggtgggccagccccagggccagggtccccttggtgaagccgtgccagggggctcagctgcttcagggaatgtgtccctcccaccatgggccagagcttcagcccttctttagctcagctagagttcacaggagagccaaaaaagaaaaggaagctgagcatctcccgagtcctgggcagggaaggggagggaaattgctgcttctccaactcttgcttggggccaagccctgcaccagttgcttcccagctgttatctgccagatcttcccatcttgtggcatgtggtgcccccaccaacatcccaaggggaccaatccccttgccaccactttgcatcacctgggaccacagatttggacaggaagggctctgagaagaggccaaagccctcattttacagatgaggaagctgaagcccggggaggggagcgaccctcaaggccacccagctggacacgggagacttgagcccagccttctgactgcattcagccctctctaggacgcagcagcctctccccagcactgagtcccccctcctttgtgtgtcccagcacccttggcctgagtaaacttggaaaggggctccctcccagagaagggactactctcttcacccctttattccagctgcctgccaccccagacccccacctcccaccctgacccccgacccctgggtggggaaggggctcacatgggcccaggctgagtgtgagtgagcatgtcaagttgtctgacactgtgacattagtgcaccctactgacaacccctccccagccttgcccctttctcctctccctgttttgtacataaattgacatgagctgcaacatgtgtgcgtgtgtgtgcgtgtgtgtgtgtgtgtatgtgtgtgtgatctgtgtcatggttttgttacctttttgtttttgtaaacttgaatgttcaaaataaacatgctgtttactctgagaaaaaaaaaaaaaaaa

SEQ ID NO: 10 - protein, 500 aa, Homo sapiens
  ref <308>: DATABASE ACCESSION NUMBER: GenBank. Accession No. NP_055108.2
MKFLAVLLAAGMLAFLGAVICIIASVPLAASPARALPGGADNASVASGAAASPGPQRSLSALHGAGGSAGPPALPGAPAASAHPLPPGPLFSRFLCTPLAAACPSGAQQGDAAGAAPGEREELLLLQSTAEQLRQTALQQEARIRADQDTIRELTGKLGRCESGLPRGLQGAGPRRDTMADGPWDSPALILELEDAVRALRDRIDRLEQELPARVNLSAAPAPVSAVPTGLHSKMDQLEGQLLAQVLALEKERVALSHSSRRQRQEVEKELDVLQGRVAELEHGSSAYSPPDAFKISIPIRNNYMYARVRKALPELYAFTACMWLRSRSSGTGQGTPFSYSVPGQANEIVLLEAGHEPMELLINDKVAQLPLSLKDNGWHHICIAWTTRDGLWSAYQDGELQGSGENLAAWHPIKPHGILILGQEQDTLGGRFDATQAFVGDIAQFNLWDHALTPAQVLGIANCTAPLLGNVLPWEDKLVEAFGGATKAAFDVCKGRAKA

SEQ ID NO: 11 - DNA, 1955 bp, Homo sapiens
  ref <308>: DATABASE ACCESSION NUMBER: GenBank. Accession No. NM_002852.3
attcatccccattcaggctttcctcagcatttattaaggactctctgctccagcctctcactctcactctcctccgctcaaactcagctcacttgagagtctcctcccgccagctgtggaaagaactttgcgtctctccagcaatgcatctccttgcgattctgttttgtgctctctggtctgcagtgttggccgagaactcggatgattatgatctcatgtatgtgaatttggacaacgaaatagacaatggactccatcccactgaggaccccacgccgtgcgcctgcggtcaggagcactcggaatgggacaagctcttcatcatgctggagaactcgcagatgagagagcgcatgctgctgcaagccacggacgacgtcctgcggggcgagctgcagaggctgcgggaggagctgggccggctcgcggaaagcctggcgaggccgtgcgcgccgggggctcccgcagaggccaggctgaccagtgctctggacgagctgctgcaggcgacccgcgacgcgggccgcaggctggcgcgtatggagggcgcggaggcgcagcgcccagaggaggcggggcgcgccctggccgcggtgctagaggagctgcggcagacgcgagccgacctgcacgcggtgcagggctgggctgcccggagctggctgccggcaggttgtgaaacagctattttattcccaatgcgttccaagaagatttttggaagcgtgcatccagtgagaccaatgaggcttgagtcttttagtgcctgcatttgggtcaaagccacagatgtattaaacaaaaccatcctgttttcctatggcacaaagaggaatccatatgaaatccagctgtatctcagctaccaatccatagtgtttgtggtgggtggagaggagaacaaactggttgctgaagccatggtttccctgggaaggtggacccacctgtgcggcacctggaattcagaggaagggctcacatccttgtgggtaaatggtgaactggcggctaccactgttgagatggccacaggtcacattgttcctgagggaggaatcctgcagattggccaagaaaagaatggctgctgtgtgggtggtggctttgatgaaacattagccttctctgggagactcacaggcttcaatatctgggatagtgttcttagcaatgaagagataagagagaccggaggagcagagtcttgtcacatccgggggaatattgttgggtggggagtcacagagatccagccacatggaggagctcagtatgtttcataaatgttgtgaaactccacttgaagccaaagaaagaaactcacacttaaaacacatgccagttgggaaggtctgaaaactcagtgcataataggaacacttgagactaatgaaagagagagttgagaccaatctttatttgtactggccaaatactgaataaacagttgaaggaaagacattggaaaaagcttttgaggataatgttactagactttatgccatggtgctttcagtttaatgctgtgtctctgtcagataaactctcaaataattaaaaaggactgtattgttgaacagagggacaattgttttacttttctttggttaattttgttttggccagagatgaattttacattggaagaataacaaaataagatttgttgtccattgttcattgttattggtatgtaccttattacaaaaaaaagatgaaaacatatttatactacaaggtgacttaacaactataaatgtagtttatgtgttataatcgaatgtcacgtttttgagaagatagtcatataagttatattgcaaaagggatttgtattaatttaagactatttttgtaaagctctactgtaaataaaatattttataaaactagctcacgtcatttaattataaatttaagagatgttttggaaaaaaaaaaaaaaaaaa

SEQ ID NO: 12 - protein, 381 aa, Homo sapiens
  ref <308>: DATABASE ACCESSION NUMBER: GenBank. Accession No. NP_002843.2
MHLLAILFCALWSAVLAENSDDYDLMYVNLDNEIDNGLHPTEDPTPCACGQEHSEWDKLFIMLENSQMRERMLLQATDDVLRGELQRLREELGRLAESLARPCAPGAPAEARLTSALDELLQATRDAGRRLARMEGAEAQRPEEAGRALAAVLEELRQTRADLHAVQGWAARSWLPAGCETAILFPMRSKKIFGSVHPVRPMRLESFSACIWVKATDVLNKTILFSYGTKRNPYEIQLYLSYQSIVFVVGGEENKLVAEAMVSLGRWTHLCGTWNSEEGLTSLWVNGELAATTVEMATGHIVPEGGILQIGQEKNGCCVGGGFDETLAFSGRLTGFNIWDSVLSNEEIRETGGAESCHIRGNIVGWGVTEIQPHGGAQYVS

SEQ ID NO: 13 - DNA, 960 bp, Homo sapiens
  ref <308>: DATABASE ACCESSION NUMBER: GenBank. Accession No. NM_001639
gggcatgaatatcagacgctagggggacagccactgtgttgtctgctaccctcatcctggtcactgcttctgctataacagccctaggccaggaatatgaacaagccgctgctttggatctctgtcctcaccagcctcctggaagcctttgctcacacagacctcagtgggaaggtgtttgtatttcctagagaatctgttactgatcatgtaaacttgatcacaccgctggagaagcctctacagaactttaccttgtgttttcgagcctatagtgatctctctcgtgcctacagcctcttctcctacaatacccaaggcagggataatgagctactagtttataaagaaagagttggagagtatagtctatacattggaagacacaaagttacatccaaagttatcgaaaagttcccggctccagtgcacatctgtgtgagctgggagtcctcatcaggtattgctgaattttggatcaatgggacacctttggtgaaaaagggtctgcgacagggttactttgtagaagctcagcccaagattgtcctggggcaggaacaggattcctatgggggcaagtttgataggagccagtcctttgtgggagagattggggatttgtacatgtgggactctgtgctgcccccagaaaatatcctgtctgcctatcagggtacccctctccctgccaatatcctggactggcaggctctgaactatgaaatcagaggatatgtcatcatcaaacccttggtgtgggtctgaggtcttgactcaacgagagcacttgaaaatgaaatgactgtctaagagatctggtcaaagcaactggatactagatcttacatctgcagctctttcttctttgaatttcctatctgtatgtctgcctaattaaaaaaatatatattgtattatgctacctgcaaaaaaaaaaaaaaaaaaaaaaaaaaaaaa

SEQ ID NO: 14 - protein, 223 aa, Homo sapiens
  ref <308>: DATABASE ACCESSION NUMBER: GenBank. Accession No. NP_001630.1
MNKPLLWISVLTSLLEAFAHTDLSGKVFVFPRESVTDHVNLITPLEKPLQNFTLCFRAYSDLSRAYSLFSYNTQGRDNELLVYKERVGEYSLYIGRHKVTSKVIEKFPAPVHICVSWESSSGIAEFWINGTPLVKKGLRQGYFVEAQPKIVLGQEQDSYGGKFDRSQSFVGEIGDLYMWDSVLPPENILSAYQGTPLPANILDWQALNYEIRGYVIIKPLVWV

SEQ ID NO: 15 - DNA, 2174 bp, Homo sapiens
  ref <308>: DATABASE ACCESSION NUMBER: GenBank. Accession No. NM_003212
aatgatagagatattagggctagttaaccacagttttacaagactcctcttcccgcgtgtgggccattgtcatgctggtgggcgtcccgcccacctgaaaggtctccccgccccgactggggtttgttgttgaagaaggagaatccccggaaaggctgagtctccagctcaaggtcaaaacgtccaaggccgaaagccctccagtttcccctggacgccttgctcctgcttctgctacgaccttctggggaaaacgaatttctcattttcttcttaaattgccattttcgctttaggagatgaatgttttcctttggctgttttggcaatgactctgaattaaagcgatgctaacgcctcttttccccctaattgttaaaagctatggactgcaggaagatggcccgcttctcttacagtgtgatttggatcatggccatttctaaagtctttgaactgggattagttgccgggctgggccatcaggaatttgctcgtccatctcggggatacctggccttcagagatgacagcatttggccccaggaggagcctgcaattcggcctcggtcttcccagcgtgtgccgcccatggggatacagcacagtaaggagctaaacagaacctgctgcctgaatgggggaacctgcatgctggggtccttttgtgcctgccctccctccttctacggacggaactgtgagcacgatgtgcgcaaagagaactgtgggtctgtgccccatgacacctggctgcccaagaagtgttccctgtgtaaatgctggcacggtcagctccgctgctttcctcaggcatttctacccggctgtgatggccttgtgatggatgagcacctcgtggcttccaggactccagaactaccaccgtctgcacgtactaccacttttatgctagttggcatctgcctttctatacaaagctactattaatcgacattgacctatttccagaaatacaattttagatatcatgcaaatttcatgaccagtaaaggctgctgctacaatgtcctaactgaaagatgatcatttgtagttgccttaaaataatgaatacatttccaaaatggtctctaacatttccttacagaactacttcttacttctttgccctgccctctcccaaaaaactacttcttttttcaaaagaaagtcagccatatctccattgtgcctaagtccagtgtttctttttttttttttttttgagacggagtctcactctgtcacccaggctggactgcaatgacgcgatcttggttcactgcaacctccgcatccggggttcaagccattctcctgcctcagcctcccaagtaactgggattacaggcatgtgtcaccatgcccagctaatttttttgtatttttagtagagatgggggtttcaccatattggccagtctggtctcgaactcctgaccttgtgatccactcgcctcagcctctcgaagtgctgagattacacacgtgagcaactgtgcaaggcctggtgtttcttgatacatgtaattctaccaaggtcttcttaatatgttcttttaaatgattgaattatatgttcagattattggagactaattctaatgtggaccttagaatacagttttgagtagagttgatcaaaatcaattaaaatagtctctttaaaaggaaagaaaacatctttaaggggaggaaccagagtgctgaaggaatggaagtccatctgcgtgtgtgcagggagactgggtaggaaagaggaagcaaatagaagagagaggttgaaaaacaaaatgggttacttgattggtgattaggtggtggtagagaagcaagtaaaaaggctaaatggaagggcaagtttccatcatctatagaaagctatataagacaagaactcccctttttttcccaaaggcattataaaaagaatgaagcctccttagaaaaaaaattatacctcaatgtccccaacaagattgcttaataaattgtgtttcctccaagctattcaattcttttaactgttgtagaagacaaaatgttcacaatatatttagttgtaaaccaagtgatcaaactacatattgtaaagcccatttttaaaatacattgtatatatgtgtatgcacagtaaaaatggaaactatattgacctaaaaaaaaaaaaa

SEQ ID NO: 16 - protein, 188 aa, Homo sapiens
  ref <308>: DATABASE ACCESSION NUMBER: GenBank. Accession No. NP_003203
MDCRKMARFSYSVIWIMAISKVFELGLVAGLGHQEFARPSRGYLAFRDDSIWPQEEPAIRPRSSQRVPPMGIQHSKELNRTCCLNGGTCMLGSFCACPPSFYGRNCEHDVRKENCGSVPHDTWLPKKCSLCKCWHGQLRCFPQAFLPGCDGLVMDEHLVASRTPELPPSARTTTFMLVGICLSIQSYY

SEQ ID NO: 17 - DNA, 1784 bp, Homo sapiens
  ref <308>: DATABASE ACCESSION NUMBER: GenBank. Accession No. NM_001174136
cctggatgttcaagggaacaatgacagagtgtgatttggatcatggccatttctaaagtctttgaactgggattagttgccgggctgggccatcaggaatttgctcgtccatctcggggatacctggccttcagagatgacagcatttggccccaggaggagcctgcaattcggcctcggtcttcccagcgtgtgccgcccatggggatacagcacagtaaggagctaaacagaacctgctgcctgaatgggggaacctgcatgctggggtccttttgtgcctgccctccctccttctacggacggaactgtgagcacgatgtgcgcaaagagaactgtgggtctgtgccccatgacacctggctgcccaagaagtgttccctgtgtaaatgctggcacggtcagctccgctgctttcctcaggcatttctacccggctgtgatggccttgtgatggatgagcacctcgtggcttccaggactccagaactaccaccgtctgcacgtactaccacttttatgctagttggcatctgcctttctatacaaagctactattaatcgacattgacctatttccagaaatacaattttagatatcatgcaaatttcatgaccagtaaaggctgctgctacaatgtcctaactgaaagatgatcatttgtagttgccttaaaataatgaatacatttccaaaatggtctctaacatttccttacagaactacttcttacttctttgccctgccctctcccaaaaaactacttcttttttcaaaagaaagtcagccatatctccattgtgcctaagtccagtgtttctttttttttttttttttgagacggagtctcactctgtcacccaggctggactgcaatgacgcgatcttggttcactgcaacctccgcatccggggttcaagccattctcctgcctcagcctcccaagtaactgggattacaggcatgtgtcaccatgcccagctaatttttttgtatttttagtagagatgggggtttcaccatattggccagtctggtctcgaactcctgaccttgtgatccactcgcctcagcctctcgaagtgctgagattacacacgtgagcaactgtgcaaggcctggtgtttcttgatacatgtaattctaccaaggtcttcttaatatgttcttttaaatgattgaattatatgttcagattattggagactaattctaatgtggaccttagaatacagttttgagtagagttgatcaaaatcaattaaaatagtctctttaaaaggaaagaaaacatctttaaggggaggaaccagagtgctgaaggaatggaagtccatctgcgtgtgtgcagggagactgggtaggaaagaggaagcaaatagaagagagaggttgaaaaacaaaatgggttacttgattggtgattaggtggtggtagagaagcaagtaaaaaggctaaatggaagggcaagtttccatcatctatagaaagctatataagacaagaactcccctttttttcccaaaggcattataaaaagaatgaagcctccttagaaaaaaaattatacctcaatgtccccaacaagattgcttaataaattgtgtttcctccaagctattcaattcttttaactgttgtagaagacaaaatgttcacaatatatttagttgtaaaccaagtgatcaaactacatattgtaaagcccatttttaaaatacattgtatatatgtgtatgcacagtaaaaatggaaactatattgacctaaaaaaaaaaaaa

SEQ ID NO: 18 - protein, 172 aa, Homo sapiens
  ref <308>: DATABASE ACCESSION NUMBER: GenBank. Accession No. NP_001167607
MAISKVFELGLVAGLGHQEFARPSRGYLAFRDDSIWPQEEPAIRPRSSQRVPPMGIQHSKELNRTCCLNGGTCMLGSFCACPPSFYGRNCEHDVRKENCGSVPHDTWLPKKCSLCKCWHGQLRCFPQAFLPGCDGLVMDEHLVASRTPELPPSARTTTFMLVGICLSIQSYY

SEQ ID NO: 19 - DNA, 1948 bp, Mus musculus
  ref <308>: DATABASE ACCESSION NUMBER: GenBank. Accession No. NM_011562
acctgagggtcctactcaacaccttaagtttcttttaagggaggtatattcctccgaagtcctcaatcacaaaccgtttataggaagaagaggccccatcccctgccggtctacacggagatcttggctgctaacttcccacagactctccaggacgggggcctctctcatttggcatatctttctttttaatctactgttttcattttgtgaaattagcctttgggtgtttcgagaatggctttatgaactaaagccatctgctaatattgtgtttcttgtcttttcctccaacgtttttacgagccgtcgaagatggggtacttctcatccagtgtggttttgcttgtggccatttccagtgcgtttgaatttggacccgttgctgggagagaccttgccatcagagataacagcatttgggaccagaaagaacctgccgtacgcgatcggtctttccagtttgtgccttccgtggggatacagaacagtaagtcgcttaataaaacttgctgtctgaatggagggacttgcatcctggggtccttctgtgcctgccctccttccttctatggacgcaactgtgaacatgatgttcgcaaagagcactgtgggtctatcctccatggcacctggctgcccaagaagtgttccctgtgcagatgctggcacggccagctccactgtcttcctcagacctttctacctggctgtgatggtcacgtgatggaccaggacctcaaagcatccgggactccgtgtcaaacgccgtctgtgacgaccacttttatgctagctggcgcctgcctttttctagatatgaaagtataggtgtcatgtgaattccatgccagtgccatagcaaagatgtcattcatcttgatgctcacagtgaatccctaatgttacccctcaaaacactaactaggccttacctctgcacagcccctcctctttctggaaaactatggcgtgtgtgccaagcactgtaacagcgagttacattcctagcctaaaagctactctaagaatgtgctgtctgccatagcctgagtttcttgatagaagtaactctcttacttttcttcctaagacttttaaacagttctgaaggttattattaaatcccaatgtgcaactggaagtaaattcagagtagctgaaaacagctaaattatctttaagcagggaggtggtggtgtctaccttaaatcaggcaaaggcaggtatatttcttgagttccaggacagacaggcctacacagaaaccctgtctcggggagaaaaaagagagagagagaagaaaaactattatcttaaaagaaaattaatggaccaggcattgtggcagatggctttaatcccagccctcaggaggtagggataggaggatctcaagagttcaaggccagcctggtctatgtagcgaacccctgtctcaaaaaggaaaaagcatcctacgagggagttgaggaagtaatgagggtctgtgcagagggagcagcagatggggaagtgactaagactggggaaacagagtggattgtttgattgatgtttacggaggtactgggaatttaaaagctaagttcttcagccaagcacagtggtgcacactattaatgtcagctctcaggaggctacaacaggactactaaaagctcatgtccaacctgggctatgtacagagtaccaggccaacaaagactgaatggcaagaccctcacagaaaacccaaacacccgagtctcttttctgcaaaagtacgaaggctttctccaagctatatacaattattcaactgttgtggaggaaagaaagtgtttattattgtagtagtaaaccagattataaaaaccacgtattgtcaagtcagtttttataataattgttcatgaatatgcacagtaaaaatgggaactctgaaaaaaaaaaaaaaaaaaaaaaa

SEQ ID NO: 20 - protein, 171 aa, Mus musculus
  ref <308>: DATABASE ACCESSION NUMBER: GenBank. Accession No. NP_035692
MGYFSSSVVLLVAISSAFEFGPVAGRDLAIRDNSIWDQKEPAVRDRSFQFVPSVGIQNSKSLNKTCCLNGGTCILGSFCACPPSFYGRNCEHDVRKEHCGSILHGTWLPKKCSLCRCWHGQLHCLPQTFLPGCDGHVMDQDLKASGTPCQTPSVTTTFMLAGACLFLDMKV

SEQ ID NO: 21 - DNA, 6947 bp, Homo sapiens
  ref <308>: DATABASE ACCESSION NUMBER: GenBank. Accession No. NM_000280
ggtgcatttgcatgttgcggagtgattagtgggtttgaaaagggaaccgtggctcggcctcatttcccgctctggttcaggcgcaggaggaagtgttttgctggaggatgatgacagaggtcaggcttcgctaatgggccagtgaggagcggtggaggcgaggccgggcgccggcacacacacattaacacacttgagccatcaccaatcagcataggaatctgagaattgctctcacacaccaacccagcaacatccgtggagaaaactctcaccagcaactcctttaaaacaccgtcatttcaaaccattgtggtcttcaagcaacaacagcagcacaaaaaaccccaaccaaacaaaactcttgacagaagctgtgacaaccagaaaggatgcctcataaagggggaagactttaactaggggcgcgcagatgtgtgaggccttttattgtgagagtggacagacatccgagatttcagagccccatattcgagccccgtggaatcccgcggcccccagccagagccagcatgcagaacagtcacagcggagtgaatcagctcggtggtgtctttgtcaacgggcggccactgccggactccacccggcagaagattgtagagctagctcacagcggggcccggccgtgcgacatttcccgaattctgcaggtgtccaacggatgtgtgagtaaaattctgggcaggtattacgagactggctccatcagacccagggcaatcggtggtagtaaaccgagagtagcgactccagaagttgtaagcaaaatagcccagtataagcgggagtgcccgtccatctttgcttgggaaatccgagacagattactgtccgagggggtctgtaccaacgataacataccaagcgtgtcatcaataaacagagttcttcgcaacctggctagcgaaaagcaacagatgggcgcagacggcatgtatgataaactaaggatgttgaacgggcagaccggaagctggggcacccgccctggttggtatccggggacttcggtgccagggcaacctacgcaagatggctgccagcaacaggaaggagggggagagaataccaactccatcagttccaacggagaagattcagatgaggctcaaatgcgacttcagctgaagcggaagctgcaaagaaatagaacatcctttacccaagagcaaattgaggccctggagaaagagtttgagagaacccattatccagatgtgtttgcccgagaaagactagcagccaaaatagatctacctgaagcaagaatacaggtatggttttctaatcgaagggccaaatggagaagagaagaaaaactgaggaatcagagaagacaggccagcaacacacctagtcatattcctatcagcagtagtttcagcaccagtgtctaccaaccaattccacaacccaccacaccggtttcctccttcacatctggctccatgttgggccgaacagacacagccctcacaaacacctacagcgctctgccgcctatgcccagcttcaccatggcaaataacctgcctatgcaacccccagtccccagccagacctcctcatactcctgcatgctgcccaccagcccttcggtgaatgggcggagttatgatacctacacccccccacatatgcagacacacatgaacagtcagccaatgggcacctcgggcaccacttcaacaggactcatttcccctggtgtgtcagttccagttcaagttcccggaagtgaacctgatatgtctcaatactggccaagattacagtaaaaaaaaaaaaaaaaaaaaaaaggaaaggaaatattgtgttaattcagtcagtgactatggggacacaacagttgagctttcaggaaagaaagaaaaatggctgttagagccgcttcagttctacaattgtgtcctgtattgtaccactggggaaggaatggacttgaaacaaggacctttgtatacagaaggcacgatatcagttggaacaaatcttcattttggtatccaaacttttattcattttggtgtattatttgtaaatgggcatttgtatgttataatgaaaaaaagaacaatgtagactggatggatgtttgatctgtgttggtcatgaagttgttttttttttttttaaaaagaaaaccatgatcaacaagctttgccacgaatttaagagttttatcaagatatatcgaatacttctacccatctgttcatagtttatggactgatgttccaagtttgtatcattcctttgcatataattaaacctggaacaacatgcactagatttatgtcagaaatatctgttggttttccaaaggttgttaacagatgaagtttatgtgcaaaaaagggtaagatataaattcaaggaagaaaaaaagttgatagctaaaaggtagagtgtgtcttcgatataatccaatttgttttatgtcaaaatgtaagtatttgtcttccctagaaatcctcagaatgatttctataataaagttaatttcatttatatttgacaagaatatagatgttttatacacattttcatgcaatcatacgtttcttttttggccagcaaaagttaattgttcttagatatagttgtattactgttcacggtccaatcattttgtgcatctagagttcattcctaatcaattaaaagtgcttgcaagagttttaaacttaagtgttttgaagttgttcacaactacatatcaaaattaaccattgttgattgtaaaaaaccatgccaaagcctttgtatttcctttattatacagttttctttttaaccttatagtgtggtgttacaaattttatttccatgttagatcaacattctaaaccaatggttactttcacacacactctgttttacatcctgatgatccttaaaaaataatccttatagataccataaatcaaaaacgtgttagaaaaaaattccacttacagcagggtgtagatctgtgcccatttatacccacaacatatatacaaaatggtaacatttcccagttagccatttaattctaaagctcaaagtctagaaataatttaaaaatgcaacaagcgattagctaggaattgttttttgaattaggactggcattttcaatctgggcagatttccattgtcagcctatttcaacaatgatttcactgaagtatattcaaaagtagatttcttaaaggagactttctgaaagctgttgcctttttcaaataggccctctcccttttctgtctccctcccctttgcacaagaggcatcatttcccattgaaccactacagctgttcccatttgaatcttgctttctgtgcggttgtggatggttggagggtggaggggggatgttgcatgtcaaggaataatgagcacagacacatcaacagacaacaacaaagcagactgtgactggccggtgggaattaaaggccttcagtcattggcagcttaagccaaacattcccaaatctatgaagcagggcccattgttggtcagttgttatttgcaatgaagcacagttctgatcatgtttaaagtggaggcacgcagggcaggagtgcttgagcccaagcaaaggatggaaaaaaataagcctttgttgggtaaaaaaggactgtctgagactttcatttgttctgtgcaacatataagtcaatacagataagtcttcctctgcaaacttcactaaaaagcctgggggttctggcagtctagattaaaatgcttgcacatgcagaaacctctggggacaaagacacacttccactgaattatactctgctttaaaaaaatccccaaaagcaaatgatcagaaatgtagaaattaatggaaggatttaaacatgaccttctcgttcaatatctactgttttttagttaaggaattacttgtgaacagataattgagattcattgctccggcatgaaatatactaataattttattccaccagagttgctgcacatttggagacaccttcctaagttgcagtttttgtatgtgtgcatgtagttttgttcagtgtcagcctgcactgcacagcagcacatttctgcaggggagtgagcacacatacgcactgttggtacaattgccggtgcagacatttctacctcctgacattttgcagcctacattccctgagggctgtgtgctgagggaactgtcagagaagggctatgtgggagtgcatgccacagctgctggctggcttacttcttccttctcgctggctgtaatttccaccacggtcaggcagccagttccggcccacggttctgttgtgtagacagcagagactttggagacccggatgtcgcacgccaggtgcaagaggtgggaatgggagaaaaggagtgacgtgggagcggagggtctgtatgtgtgcacttgggcacgtatatgtgtgctctgaaggtcaggattgccagggcaaagtagcacagtctggtatagtctgaagaagcggctgctcagctgcagaagccctctggtccggcaggatgggaacggctgccttgccttctgcccacaccctagggacatgagctgtccttccaaacagagctccaggcactctcttggggacagcatggcaggctctgtgtggtagcagtgcctgggagttggccttttactcattgttgaaataatttttgtttattatttatttaacgatacatatatttatatatttatcaatggggtatctgcagggatgttttgacaccatcttccaggatggagattatttgtgaagacttcagtagaatcccaggactaaacgtctaaattttttctccaaacttgactgacttgggaaaaccaggtgaatagaataagagctgaatgttttaagtaataaacgttcaaactgctctaagtaaaaaaatgcattttactgcaatgaatttctagaatatttttcccccaaagctatgcctcctaacccttaaatggtgaacaactggtttcttgctacagctcactgccatttcttcttactatcatcactaggtttcctaagattcactcatacagtattatttgaagattcagctttgttctgtgaatgtcatcttaggattgtgtctatattcttttgcttatttctttttactctgggcctctcatactagtaagattttaaaaagccttttcttctctgtatgtttggctcaccaaggcgaaatatatattcttctctttttcatttctcaagaataaacctcatctgcttttttgtttttctgtgttttggcttggtactgaatgactcaactgctcggttttaaagttcaaagtgtaagtacttagggttagtactgcttatttcaataatgttgacggtgactatctttggaaagcagtaacatgctgtcttagaaatgacattaataatgggcttaaacaaatgaataggggggtccccccactctccttttgtatgcctatgtgtgtctgatttgttaaaagatggacagggaattgattgcagagtgtcgcttccttctaaagtagttttattttgtctactgttagtatttaaagatcctggaggtggacataaggaataaatggaagagaaaagtagatattgtatggtggctactaaaaggaaattcaaaaagtcttagaacccgagcacctgagcaaactgcagtagtcaaaatatttatctcatgttaaagaaaggcaaatctagtgtaagaaatgagtaccatatagggttttgaagttcatatactagaaacacttaaaagatatcatttcagatattacgtttggcattgttcttaagtatttatatctttgagtcaagctgataattaaaaaaaatctgttaatggagtgtatatttcataatgtatcaaaatggtgtctatacctaaggtagcattattgaagagagatatgtttatgtagtaagttattaacataatgagtaacaaataatgtttccagaagaaaggaaaacacattttcagagtgcgtttttatcagaggaagacaaaaatacacacccctctccagtagcttatttttacaaagccggcccagtgaattagaaaaacaaagcacttggatatgatttttggaaagcccaggtacacttattattcaaaatgcacttttactgagtttgaaaagtttcttttatatttaaaataagggttcaaatatgcatattcaatttttatagtagttatctatttgcaaagcatatattaactagtaattggctgttaattttatagacatggtagccagggaagtatatcaatgacctattaagtattttgacaagcaatttacatatctgatgacctcgtatctctttttcagcaagtcaaatgctatgtaattgttccattgtgtgttgtataaaatgaatcaacacggtaagaaaaaggttagagttattaaaataataaactgactaaaatactcatttgaatttattcagaatgttcataatgctttcaaaggacatagcagagcttttgtggagtatccgcacaacattatttattatctatggactaaatcaattttttgaagttgctttaaaatttaaaagcacctttgcttaatataaagccctttaattttaactgacagatcaattctgaaactttattttgaaaagaaaatggggaagaatctgtgtctttagaattaaaagaaatgaaaaaaataaacccgacattctaaaaaaatagaataagaaacctgatttttagtactaatgaaatagcgggtgacaaaatagttgtctttttgattttgatcacaaaaaataaactggtagtgacaggatatgatggagagatttgacatcctggcaaatcactgtcattgattcaattattctaattctgaataaaagctgtatacagtaaaa

SEQ ID NO: 22 - DNA, 4108 bp, Homo sapiens
  ref <308>: DATABASE ACCESSION NUMBER: GenBank Accession No.: NM_ 005986.2
ccggccgtctatgctccaggccctctcctcgcggtgccggtgaacccgccagccgccccgatgtacagcatgatgatggagaccgacctgcactcgcccggcggcgcccaggcccccacgaacctctcgggccccgccggggcgggcggcggcgggggcggaggcgggggcggcggcggcggcgggggcgccaaggccaaccaggaccgggtcaaacggcccatgaacgccttcatggtgtggtcccgcgggcagcggcgcaagatggcccaggagaaccccaagatgcacaactcggagatcagcaagcgcctgggggccgagtggaaggtcatgtccgaggccgagaagcggccgttcatcgacgaggccaagcggctgcgcgcgctgcacatgaaggagcacccggattacaagtaccggccgcgccgcaagaccaagacgctgctcaagaaggacaagtactcgctggccggcgggctcctggcggccggcgcgggtggcggcggcgcggctgtggccatgggcgtgggcgtgggcgtgggcgcggcggccgtgggccagcgcctggagagcccaggcggcgcggcgggcggcggctacgcgcacgtcaacggctgggccaacggcgcctaccccggctcggtggcggcggcggcggcggccgcggccatgatgcaggaggcgcagctggcctacgggcagcacccgggcgcgggcggcgcgcacccgcacgcgcaccccgcgcacccgcacccgcaccacccgcacgcgcacccgcacaacccgcagcccatgcaccgctacgacatgggcgcgctgcagtacagccccatctccaactcgcagggctacatgagcgcgtcgccctcgggctacggcggcctcccctacggcgccgcggccgccgccgccgccgctgcgggcggcgcgcaccagaactcggccgtggcggcggcggcggcggcggcggccgcgtcgtcgggcgccctgggcgcgctgggctctctggtgaagtcggagcccagcggcagcccgcccgccccagcgcactcgcgggcgccgtgccccggggacctgcgcgagatgatcagcatgtacttgcccgccggcgaggggggcgacccggcggcggcagcagcggccgcggcgcagagccggctgcactcgctgccgcagcactaccagggcgcgggcgcgggcgtgaacggcacggtgcccctgacgcacatctagcgccttcgggacgccggggactctgcggcggcgacccacgagctcgcggcccgcgcccggctcccgccccgccccggcgcggcgtggcttttgtacagacgttcccacattcttgtcaaaaggaaaatactggagacgaacgccgggtgacgcgtgtcccccactcaccttccccggagaccctggcgaccgccgggcgctgacaccagacttgggttttagactgaacttcggtgttttcttgagactttttgtacagtatttatcacctacggaggaagcggaaagcgttttctttgctcgaggggacaaaaaagtcaaaacgaggcgagaggcgaagcccacttttgtataccggccggcgcgctcactttcctccgcgttgcttccggacggcgccgaccgccggagcccaagtgacgcggagctcgtcgcatttgttataaatgtagtaaggcaggtccaagcacttacaagttttttgtagttgttaccgctcttttgggttggtttgttaatttatacaaagagattaccaccaccaccccctccttcagacggcggagttatattctgggttttgtaaaactttatgtatctgagcatttccatttttttttttgggttttgtattatttcttgtaaatgcattgtgaaaaattttattttcggcgttgcaatgcggggaggagaagtcagattatgtacatagttttctaaaaagcctttcttctaaaaacgaaaaaagaccccccacccaaaatgtttcgagtcaacaaatttaagagacagagcccattttctccataaatttgtaacatgctatttttatgtgcatgttttatgagttcaaaatgcaatgaggaaatctgacagggaaattatctgtatgaactaaaagtaagggaaccccggggaatgggaggacaggatttttcaaggaacctttttcaatgaaagagaaggaagttaaaacctataggttattttgtagagctgagtgttaatacgggccgagaaataaaagtatcttctgctccggctgtttcactgcggacggctggggctgctgcgcgttaccttgctgcaagcggggcgccttccacctggctgggggtctgcgccacagtttggtccagaggagggaggaggaagggaagaccccagtggtgggaccctggaccaggccatggatgaaggacaaagaccagggcaggtcacgggtttcccaattccccagcaattaagatttcgagcagaatttatctaaatgtgtttcaaggaaacacaatcgctgaaccaaaacgtactgcagccgagccccctccgtccatcctctgcccctccccctggcttctttctcttgggaaaacgggcaaaataattgtgctggattctcacacacacagaaatatcgaccatcaccctcccccgcgtgaactgggatgcaagttgctaaccgatgtgaacgcaaaatgccttgttcattattcctgacgagatcttgaggttgtttgatgctttaaattttttaattatattattttctaggtgtttattggtacattgcagttttttttttgaaatttaaaaatttctgtaaaactttgtcttcaagtaatctgacagcattaaatattgcatttaaaaattatactgtagcaaatacatttaaaaattaatcacaacgttaagatgaaattatatttttggaaaaaaaaaacacttgaagcccagatggaaatacgtttatttcagcagccttaggtttcccctcgctttctcaacacccttccttgtcctggagtatggactgtccgtccaaaagtgagcctatgctataagtttaatgagaaccgaattcagcctgcattcgagaatagctttaagtataatgctgatctgacaattgacgtgtaatttgggaagtcattttgataattttgcttaaaccactcattcgttaaagtgattacaaaaaagttcaagaatgatgtccactgctttctaacaagataataaaccccccccctcttttctttttctttatttttatttcttttagctatttgatcctttctgaagcagttgtttctggaagagtctgtgcgcccatggatggctgagcaccactacgacttagtccgggataagggcctccccagtcctctccgggagatgatttgggaaattttataatgcttgttctgttaactcaccgggaccttgagggtccaatgggaccttgagggttttctctgaaatatacaaacttaaaggactctctctgaggttctttgactgacgtccactctcagtctggcccctgtgctcccctgtgtgtaccctggagtttctgtgtccaattgttggcatctaggtcttggctcaagattaggatgtgggccccactttagaggcacagactatgaaaagctgagttagtgcgcccgggacgccaggcaagcagcttttacagtttggcatcttattgcaggtgcttcgtgcacagtcagctgaaatagccaatgccaggtgctccaaccaccttatttccttgttttgttgattagaacaacacagaaaaaagcaaatataaatttttaatgactccatttaaaaatatcacagggtgggggcaaggaaattagctgagattcatctcaggattgagattctatccccccttccccgcccccagcagtgtcgctccaattcaaattagtggagaaaagattacagtaggccctgagccgactgtgaattcggtgcttggccaaggtaacactcatcgtattcacggagtgaaatactatatgatgatagttattatattatatgacgacttcattcacttcccaaatcacagggt

SEQ ID NO: 23 - DNA, 2098 bp, Homo sapiens
  ref <308>: DATABASE ACCESSION NUMBER: GenBank Accession No.: NM_024865.2
attataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtgaagatgagtgaaactgatattactcaatttcagtctggacactggctgaatccttcctctcccctcctcccatccctcataggatttttcttgtttggaaaccacgtgttctggtttccatgatgcccatccagtcaatctcatggagggtggagtatggttggagcctaatcagcgaggtttctttttttttttttttcctattggatcttcctggagaaaatactttttttttttttttttttgaaacggagtcttgctctgtcgcccaggctggagtgcagtggcgcggtcttggctcactgcaagctccgtctcccgggttcacgccattctcctgcctcagcctcccgagcagctgggactacaggcgcccgccacctcgcccggctaatattttgtatttttagtagagacggggtttcactgtgttagccaggatggtctcgatctcctgaccttgtgatccacccgcctcggcctccctaacagctgggatttacaggcgtgagccaccgcgccctgcctagaaaagacattttaataaccttggctgccgtctctggctatagataagtagatctaatactagtttggatatctttagggtttagaatctaacctcaagaataagaaatacaagtacaaattggtgatgaagatgtattcgtattgtttgggattgggaggctttgcttattttttaaaaactattgaggtaaagggttaagctgtaacatacttaattgatttcttaccgtttttggctctgttttgctatatcccctaatttgttggttgtgctaatctttgtagaaagaggtctcgtatttgctgcatcgtaatgacatgagtactgctttagttggtttaagttcaaatgaatgaaacaactatttttcctttagttgattttaccctgatttcaccgagtgtttcaatgagtaaatatacagcttaaacat

SEQ ID NO: 24 - DNA, 2949 bp, Homo sapiens
  ref <308>: DATABASE ACCESSION NUMBER: GenBank Accession No.: NM_004235.4
agtttcccgaccagagagaacgaacgtgtctgcgggcgcgcggggagcagaggcggtggcgggcggcggcggcaccgggagccgccgagtgaccctcccccgcccctctggccccccaccctcccacccgcccgtggcccgcgcccatggccgcgcgcgctccacacaactcaccggagtccgcgccttgcgccgccgaccagttcgcagctccgcgccacggcagccagtctcacctggcggcaccgcccgcccaccgccccggccacagcccctgcgcccacggcagcactcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctgaaaaaaaaaaaaaaaaaa

SEQ ID NO: 25 - DNA, 6055 bp, Homo sapiens
  ref <308>: DATABASE ACCESSION NUMBER: GenBank Accession No.: NM_002253
actgagtcccgggaccccgggagagcggtcaatgtgtggtcgctgcgtttcctctgcctgcgccgggcatcacttgcgcgccgcagaaagtccgtctggcagcctggatatcctctcctaccggcacccgcagacgcccctgcagccgcggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaaaaggaagcatccacacccccaactcctggacatcacatgagaggtgctgctcagattttcaagtgttgttctttccaccagcaggaagtagccgcatttgattttcatttcgacaacagaaaaaggacctcggactgcagggagccagtcttctaggcatatcctggaagaggcttgtgacccaagaatgtgtctgtgtcttctcccagtgttgacctgatcctctttttcattcatttaaaaagcatttatcatgccccctgctgcgggtctcaccatgggtttagaacaaagacgttcaagaaatggccccatcctcaaagaagtagcagtacctggggagctgacacttctgtaaaactagaagataaaccaggcaatgtaagtgttcgaggtgttgaagatgggaaggatttgcagggctgagtctatccaagaggctttgtttaggacgtgggtcccaagccaagccttaagtgtggaattcggattgatagaaaggaagactaacgttaccttgctttggagagtactggagcctgcaaatgcattgtgtttgctctggtggaggtgggcatggggtctgttctgaaatgtaaagggttcagacggggtttctggttttagaaggttgcgtgttcttcgagttgggctaaagtagagttcgttgtgctgtttctgactcctaatgagagttccttccagaccgttacgtgtctcctggccaagccccaggaaggaaatgatgcagctctggctccttgtctcccaggctgatcctttattcagaataccacaaagaaaggacattcagctcaaggctccctgccgtgttgaagagttctgactgcacaaaccagcttctggtttcttctggaatgaataccctcatatctgtcctgatgtgatatgtctgagactgaatgcgggaggttcaatgtgaagctgtgtgtggtgtcaaagtttcaggaaggattttacccttttgttcttccccctgtccccaacccactctcaccccgcaacccatcagtattttagttatttggcctctactccagtaaacctgattgggtttgttcactctctgaatgattattagccagacttcaaaattattttatagcccaaattataacatctattgtattatttagacttttaacatatagagctatttctactgatttttgcccttgttctgtcctttttttcaaaaaagaaaatgtgttttttgtttggtaccatagtgtgaaatgctgggaacaatgactataagacatgctatggcacatatatttatagtctgtttatgtagaaacaaatgtaatatattaaagccttatatataatgaactttgtactattcacattttgtatcagtattatgtagcataacaaaggtcataatgctttcagcaattgatgtcattttattaaagaacattgaaaaacttgaaggaatccctttgcaaggttgcattactgtacccatcatttctaaaatggaagagggggtggctgggcacagtggccgacacctaaaaacccagcactttggggggccaaggtgggaggatcgcttgagcccaggagttcaagaccagtctggccaacatggtcagattccatctcaaagaaaaaaggtaaaaataaaataaaatggagaagaaggaatcaga

SEQ ID NO: 26 - DNA, 5190 bp, Homo sapiens
  ref <308>: DATABASE ACCESSION NUMBER: GenBank Accession No.: NM_000222.2
tctgggggctcggctttgccgcgctcgctgcacttgggcgagagctggaacgtggaccagagctcggatcccatcgcagctaccgcgatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatctagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaacaaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttggatattcttgaaagtttatatttttataattttttcttacatcagatgtttctttgcagtggcttaatgtttgaaattattttgtggctttttttgtaaatattgaaatgtagcaataatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattctaataaaatgtacatatataaatcaaaaaaaaaaaaaaaa

SEQ ID NO: 27 - DNA, 2350 bp, Homo sapiens
  ref <308>: DATABASE ACCESSION NUMBER: GenBank Accession No.: NM_022454.3
gcagtgtcactaggccggctgggggccctgggtacgctgtagaccagaccgcgacaggccagaacacgggcggcggcttcgggccgggagacccgcgcagccctcggggcatctcagtgcctcactccccaccccctcccccgggtcgggggaggcggcgcgtccggcggagggttgaggggagcggggcaggcctggagcgccatgagcagcccggatgcgggatacgccagtgacgaccagagccagacccagagcgcgctgcccgcggtgatggccgggctgggcccctgcccctgggccgagtcgctgagccccatcggggacatgaaggtgaagggcgaggcgccggcgaacagcggagcaccggccggggccgcgggccgagccaagggcgagtcccgtatccggcggccgatgaacgctttcatggtgtgggctaaggacgagcgcaagcggctggcgcagcagaatccagacctgcacaacgccgagttgagcaagatgctgggcaagtcgtggaaggcgctgacgctggcggagaagcggcccttcgtggaggaggcagagcggctgcgcgtgcagcacatgcaggaccaccccaactacaagtaccggccgcggcggcgcaagcaggtgaagcggctgaagcgggtggagggcggcttcctgcacggcctggctgagccgcaggcggccgcgctgggccccgagggcggccgcgtggccatggacggcctgggcctccagttccccgagcagggcttccccgccggcccgccgctgctgcctccgcacatgggcggccactaccgcgactgccagagtctgggcgcgcctccgctcgacggctacccgttgcccacgcccgacacgtccccgctggacggcgtggaccccgacccggctttcttcgccgccccgatgcccggggactgcccggcggccggcacctacagctacgcgcaggtctcggactacgctggccccccggagcctcccgccggtcccatgcacccccgactcggcccagagcccgcgggtccctcgattccgggcctcctggcgccacccagcgcccttcacgtgtactacggcgcgatgggctcgcccggggcgggcggcgggcgcggcttccagatgcagccgcaacaccagcaccagcaccagcaccagcaccaccccccgggccccggacagccgtcgccccctccggaggcactgccctgccgggacggcacggaccccagtcagcccgccgagctcctcggggaggtggaccgcacggaatttgaacagtatctgcacttcgtgtgcaagcctgagatgggcctcccctaccaggggcatgactccggtgtgaatctccccgacagccacggggccatttcctcggtggtgtccgacgccagctccgcggtatattactgcaactatcctgacgtgtgacaggtccctgatccgccccagcctgcaggccagaagcagtgttacacacttcctggaggagctaaggaaatcctcagactcctgggtttttgttgttgctgttgttgttttttaaaaggtgtgttggcatataatttatggtaatttattttgtctgccacttgaacagtttgggggggtgaggtttcatttaaaatttgttcagagatttgtttcccatagttggattgtcaaaaccctatttccaagttcaagttaactagctttgaatgtgtcccaaaacagcttcctccatttcctgaaagtttattgatcaaagaaatgttgtcctgggtgtgttttttcaatcttctaaaaaataaaatctggaatcctgcttttttgctctactagtacctctgtcacactagtcttatcaaaaaccagttcttaagatcaatgttaagtttattagttaatgtaaatttctcatcctcgaaaagggtgaacataaatgcctttaaggagtatatctaaaaataaacattaggatatctaagtttgatgtaattgtttcaggaaggaaaaaagaaaagcattctggaatgagcctacttcaagtaatcttagtttctaaaactaacagttaatattttcaattccagtatatcactttaagtagaaggggatgtccaagtaattttggttttctaactgttgaatcataagcttgacctgcccccagaggctttttggatgtttttatctgtgttttgccatctctttacactcctcgacattcagtttaccttaatcttcacatttttacaccttgggaagtggcaagcatcgctgggtttaagataaaggagtcacaaaaactaatcaaaataaaatttgcattatgacaacttttaataca

SEQ ID NO: 28 - DNA, 3056 bp, Homo sapiens
  ref <308>: DATABASE ACCESSION NUMBER: GenBank Accession No.: NM_005900.2
ggcagctgaggagtggaggctgggcagctccgactccctgacgccagcgcgaccagatcaatccaggctccaggagaaagcaggcgggcgggcggagaaaggagaggccgagcggctcaacccgggccgaggctcggggagcggagagtggcgcagcgcccggccgtccggacccgggccgcgagaccccgctcgcccggccactcgtgctcccacacggacgggcgcgccgccaacccggtgctgactgggttacttttttaaacactaggaatggtaatttctactcttctggacttcaaactaagaagttaaagagacttctctgtaaataaacaaatctcttctgctgtccttttgcatttggagacagctttatttcaccatatccaaggagtataactagtgctgtcattatgaatgtgacaagtttattttcctttacaagtccagctgtgaagagacttcttgggtggaaacagggcgatgaagaagaaaaatgggcagagaaagctgttgatgctttggtgaaaaaactgaagaaaaagaaaggtgccatggaggaactggaaaaggccttgagctgcccagggcaaccgagtaactgtgtcaccattccccgctctctggatggcaggctgcaagtctcccaccggaagggactgcctcatgtcatttactgccgtgtgtggcgctggcccgatcttcagagccaccatgaactaaaaccactggaatgctgtgagtttccttttggttccaagcagaaggaggtctgcatcaatccctaccactataagagagtagaaagccctgtacttcctcctgtgctggttccaagacacagcgaatataatcctcagcacagcctcttagctcagttccgtaacttaggacaaaatgagcctcacatgccactcaacgccacttttccagattctttccagcaacccaacagccacccgtttcctcactctcccaatagcagttacccaaactctcctgggagcagcagcagcacctaccctcactctcccaccagctcagacccaggaagccctttccagatgccagctgatacgcccccacctgcttacctgcctcctgaagaccccatgacccaggatggctctcagccgatggacacaaacatgatggcgcctcccctgccctcagaaatcaacagaggagatgttcaggcggttgcttatgaggaaccaaaacactggtgctctattgtctactatgagctcaacaatcgtgtgggtgaagcgttccatgcctcctccacaagtgtgttggtggatggtttcactgatccttccaacaataagaaccgtttctgccttgggctgctctccaatgttaaccggaattccactattgaaaacaccaggcggcatattggaaaaggagttcatctttattatgttggaggggaggtgtatgccgaatgccttagtgacagtagcatctttgtgcaaagtcggaactgcaactaccatcatggatttcatcctactactgtttgcaagatccctagtgggtgtagtctgaaaatttttaacaaccaagaatttgctcagttattggcacagtctgtgaaccatggatttgagacagtctatgagcttacaaaaatgtgtactatacgtatgagctttgtgaagggctggggagcagaataccaccgccaggatgttactagcaccccctgctggattgagatacatctgcacggccccctccagtggctggataaagttcttactcaaatgggttcacctcataatcctatttcatctgtatcttaaatggccccaggcatctgcctctggaaaactattgagccttgcatgtacttgaaggatggatgagtcagacacgattgagaactgacaaaggagccttgataatacttgacctctgtgaccaactgttggattcagaaatttaaacaaaaaaaaaaaaaaacacacacaccttggtaacatactgttgatatcaagaacctgtttagtttacattgtaacattctattgtaaaatcaactaaaattcagacttttagcaggactttgtgtacagttaaaggagagatggccaagccagggacaaattgtctattagaaaacggtcctaagagattctttggtgtttggcactttaaggtcatcgttgggcagaagtttagcattaatagttgttctgaaacgtgttttatcaggtttagagcccatgttgagtcttcttttcatgggttttcataatattttaaaactatttgtttagcgatggttttgttcgtttaagtaaaggttaatcttgatgatatacataataatctttctaaaattgtatgctgaccatacttgctgtcagaataatgctaggcatatgctttttgctaaatatgtatgtacagagtatttggaagttaagaattgattagactagtgaatttaggagtatttgaggtgggtggggggaagagggaaatgacaactgcaaatgtagactatactgtaaaaattcagtttgttgctttaaagaaacaaactgatacctgaattttgctgtgtttccattttttagagatttttatcatttttttctctctcggcattcttttttctcatactcttcaaaaagcagttctgcagctggttaattcatgtaactgtgagagcaaatgaataattcctgctattctgaaattgcctacatgtttcaataccagttatatggagtgcttgaatttaataagcagtttttacggagtttacagtacagaaataggctttaattttcaagtgaattttttgccaaacttagtaactctgttaaatatttggaggatttaaagaacatcccagtttgaattcatttcaaactttttaaatttttttgtactatgtttggttttattttccttctgttaatcttttgtattcacttatgctctcgtacattgagtacttttattccaaaactagtgggttttctctactggaaattttcaataaacctgtcattattgcttactttgattaaaaa

SEQ ID NO: 29 - DNA, 10384 bp, Homo sapiens
  ref <308>: DATABASE ACCESSION NUMBER: GenBank Accession No.: NM_005901.4
gcgcccgggccgccggccgggcccgggcctgggggcggggcgggaagacggcggccgggagtgttttcagttccgcctccaatcgcccattcccctcttcccctcccagccccctccatcccatcggaagaggaaggaacaaaaggtcccggaccccccggatctgacggggcgggacctggcgccaccttgcaggttcgatacaagaggctgttttcctagcgtggcttgctgcctttggtaagaacatgtcgtccatcttgccattcacgccgccagttgtgaagagactgctgggatggaagaagtcagctggtgggtctggaggagcaggcggaggagagcagaatgggcaggaagaaaagtggtgtgagaaagcagtgaaaagtctggtgaagaagctaaagaaaacaggacgattagatgagcttgagaaagccatcaccactcaaaactgtaatactaaatgtgttaccataccaagcacttgctctgaaatttggggactgagtacaccaaatacgatagatcagtgggatacaacaggcctttacagcttctctgaacaaaccaggtctcttgatggtcgtctccaggtatcccatcgaaaaggattgccacatgttatatattgccgattatggcgctggcctgatcttcacagtcatcatgaactcaaggcaattgaaaactgcgaatatgcttttaatcttaaaaaggatgaagtatgtgtaaacccttaccactatcagagagttgagacaccagttttgcctccagtattagtgccccgacacaccgagatcctaacagaacttccgcctctggatgactatactcactccattccagaaaacactaacttcccagcaggaattgagccacagagtaattatattccagaaacgccacctcctggatatatcagtgaagatggagaaacaagtgaccaacagttgaatcaaagtatggacacaggctctccagcagaactatctcctactactctttcccctgttaatcatagcttggatttacagccagttacttactcagaacctgcattttggtgttcgatagcatattatgaattaaatcagagggttggagaaaccttccatgcatcacagccctcactcactgtagatggctttacagacccatcaaattcagagaggttctgcttaggtttactctccaatgttaaccgaaatgccacggtagaaatgacaagaaggcatataggaagaggagtgcgcttatactacataggtggggaagtttttgctgagtgcctaagtgatagtgcaatctttgtgcagagccccaattgtaatcagagatatggctggcaccctgcaacagtgtgtaaaattccaccaggctgtaatctgaagatcttcaacaaccaggaatttgctgctcttctggctcagtctgttaatcagggttttgaagccgtctatcagctaactagaatgtgcaccataagaatgagttttgtgaaagggtggggagcagaataccgaaggcagacggtaacaagtactccttgctggattgaacttcatctgaatggacctctacagtggttggacaaagtattaactcagatgggatccccttcagtgcgttgctcaagcatgtcataaagcttcaccaatcaagtcccatgaaaagacttaatgtaacaactcttctgtcatagcattgtgtgtggtccctatggactgtttactatccaaaagttcaagagagaaaacagcacttgaggtctcatcaattaaagcaccttgtggaatctgtttcctatatttgaatattagatgggaaaattagtgtctagaaatactctcccattaaagaggaagagaagattttaaagacttaatgatgtcttattgggcataaaactgagtgtcccaaaggtttattaataacagtagtagttatgtgtacaggtaatgtatcatgatccagtatcacagtattgtgctgtttatatacatttttagtttgcatagatgaggtgtgtgtgtgcgctgcttcttgatctaggcaaacctttataaagttgcagtacctaatctgttattcccacttctctgttatttttgtgtgtcttttttaatatataatatatatcaagattttcaaattatttagaagcagattttcctgtagaaaaactaatttttctgccttttaccaaaaataaactcttgggggaagaaaagtggattaacttttgaaatccttgaccttaatgtgttcagtggggcttaaacagtcattctttttgtggttttttgtttttttttgtttttttttttaactgctaaatcttattataaggaaaccatactgaaaacctttccaagcctcttttttccattcccatttttgtcctcataatcaaaacagcataacatgacatcatcaccagtaatagttgcattgatactgctggcaccagttaattctgggatacagtaagaattcatatggagaaagtccctttgtcttatgcccaaatttcaacaggaataattggcttgtataatctagcagtctgttgatttatccttccacctcataaaaaatgcataggtggcagtataattattttcagggatatgctagaattacttccacatatttatccctttttaaaaaagctaatctataaataccgtttttccaaaggtattttacaatatttcaacagcagaccttctgctcttcgagtagtttgatttggtttagtaaccagattgcattatgaaatgggccttttgtaaatgtaattgtttctgcaaaatacctagaaaagtgatgctgaggtaggatcagcagatatgggccatctgtttttaaagtatgttgtattcagtttataaattgattgttattctacacataattatgaattcagaattttaaaaattgggggaaaagccatttatttagcaagttttttagcttataagttacctgcagtctgagctgttcttaactgatcctggttttgtgattgacaatatttcatgctctgtagtgagaggagatttccgaaactctgttgctagttcattctgcagcaaataattattatgtctgatgttgactcattgcagtttaaacatttcttcttgtttgcatcttagtagaaatggaaaataaccactcctggtcgtcttttcataaattttcatatttttgaagctgtctttggtacttgttctttgaaatcatatccacctgtctctataggtatcattttcaatactttcaacatttggtggttttctattgggtactccccattttcctatatttgtgtgtatatgtatgtgttcatgtaaatttggtatagtaattttttattcattcaacaaatatttattgttcacctgtttgtaccaggaacttttcttagtctttgggtaaaggtgaacaagacaactacagttcctgcctttgctgagacagcagttacactaacccttaattatcttacttgtctatgaaggagataaacagggtactgtactggagaataacagatgggatgcttcaggtaggacatcaaggaaagcctctaaggaaaggatgcatgagctaacacctgacattaaagaagcaagccaagtgaggagccaggggagataagcattcctggcaaagagaatagcatcaaatgcaaaaaggttcacactaaaggaaactcctgattaggtattaatgctttatacagaaacctctatacaaatccaaacttgaagatcagaatggttctacagttcataacattttgaaggtggccttattttgtgatagtctgcttcatgtgattctcactaacatatctccttcctcaacctttgctgtaaaaatttcatttgcaccacatcagtactacttaatttaacaagcttttgttgtgtaagctctcactgttttagtgccctgctgcttgcttccagactttgtgctgtccagtaattatgtcttccactacccatcttgtgagcagagtaaatgtcctaggtaataccactatcaggcctgtaggagatactcagtggagcctctgcccttctttttcttacttgagaacttgtaatggtgttagggaacagttgtaggggcagaaaacaactctgaaagtggtagaaggtcctgatcttggtggttactcttgcattactgtgttaggtcaagcagtgcctactatgctgtttcagtagtggagcgcatctctacagttctgatgcgatttttctgtacagtatgaaattgggactcaactctttgaaaacacctattgagcagttatacctgttgagcagtttacttcctggttgtaattacatttgtgtgaatgtgtttgatgctttttaacgagatgatgttttttgtattttatctactgtggcctgattttttttttgttttctgcccctccccccatttataggtgtggttttcatttttctaagtgatagaatcccctctttgttgaatttttgtctttatttaaattagcaacattacttaggatttattcttcacaatactgttaattttctaggaatgatgacctgagaaccgaatggccatgctttctatcacatttctaagatgagtaatattttttccagtaggttccacagagacaccttgggggctggcttaggggaggctgttggagttctcactgacttagtggcatatttattctgtactgaagaactgcatggggtttcttttggaaagagtttcattgctttaaaaagaagctcagaaagtctttataaccactggtcaacgattagaaaaatataactggatttaggcctaccttctggaataccgctgattgtgctctttttatcctactttaaagaagctttcatgattagatttgagctatatcagttataccgattataccttataatacacattcagttagtaaacatttattgatgcctgttgtttgcccagccactgtgatggatattgaataataaaaagatgactaggacggggccctgacccttgagctgtgcttggtcttgtagaggttgtgttttttttcctcaggacctgtcactttggcagaaggaaatctgcctaatttttcttgaaagctaaattttctttgtaagtttttacaaattgtttaatacctagttgtattttttaccttaagccacattgagttttgcttgatttgtctgtcttttaaacactgtcaaatgctttcccttttgttaaaattattttaatttcactttttttgtgcccttgtcaatttaagactaagactttgaaggtaaaacaaacaaacaaacatcagtcttagtctcttgctagttgaaatcaaataaaagaaaatatatacccagttggtttctctacctcttaaaagcttcccatatatacctttaagatccttctcttttttctttaactactaaataggttcagcatttattcagtgttagataccctcttcgtctgagggtggcgtaggtttatgttgggatataaagtaacacaagacaatcttcactgtacataaaatatgtcttcatgtacagtctttactttaaaagctgaacattccaatttgcgccttccctcccaagcccctgcccaccaagtatctctttagatatctagtctgtggacatgaacaatgaatacttttttcttactctgatcgaaggcattgatacttagacatatcaaacatttcttcctttcatatgctttactttgctaaatctattatattcattgcctgaattttattcttcctttctacctgacaacacacatccaggtggtacttgctggttatcctctttcttgttagccttgttttttgttttttttttttttttttgagagggagtctcgctctgttgcccaacctggagtgcagtggtgcgatcttggttcactgcaagctccgcctcccgggttcacgccatgcttctgcctcagcctcccaagtagctgggactacaggcgcccaccaccacactcggctaattttttgtatttttagtagagacggggtttcaccgtgttggccaggatggtctcgatctcctgacctcgtgatctgtccacctcggcttcccaaagtgctgggattacaggcatgagccaccgcgcccagcctagccatatttttatctgcatatatcagaatgtttctctcctttgaacttattaacaaaaaaggaacatgcttttcatacctagagtcctaatttcttcatcatgaaggttgctattcaaattgatcaatcattttaattttacaaatggctcaaaaattctgttcagtaaatgtctttgtgactggcaaatggcataaattatgtttaagattatgaacttttctgacagttgcagccaatgttttccctacgataccagatttccatcttggggcatattggattgttgtatttaagacagtcagaataatgatagtgtgtggtctccagaggtagtcagaatcctgctattgagttctttttatatcttccttttcaattttttattaccattttgtttgtttagactacactttgtagggattgaggggcaaattatctcttggagtggaattcctgtgttttgagccttacaaccaggaaatatgagctatactagatagcctcatgatagcatttacgataagaacttatctcgtgtgttcatgtaattttttgagtaggaactgttttatcttgaatattgtagctaactatatatagcagaactgcctcagtctttttaagaaggaaataaataatatatgtgtatgaatttatatatacatatacactcatagacaaacttaacagttggggtcattctaacagttaaaacaattgttccattgtttaaatctcagatcctggtaaaatgttcttaatttgtctgtgtacattttcctttcatggacagaccattggagtacattaattttcttaatctgccatttggcagttcatttaatataccattttttggcaacttggtaactaagaatcacagccaaaatttgttaacatcaaagaaagctctgccatataccccgttactaaattattatacatccagcagattctgggatgtactaacttagggttaactttgttgttgttgataatactagattgctccctctttaattcttcttctggtgcaaggttgctgcttaagttaccctgggaaatactactacaaggtcaaattttctagtatcttacagcctgattgaaggtgattcagatctttgctcaatataaatggattttccaagattctctgggccatccttgacccacaggtgatctcgctggagtatattaacttaacttcagtgccagttggtttggtgccatgagatccataatgaatccagaacttcaccattgcttagatataagagtcccttggaagaataatgccactgatgatgggggtcagaaggtgtattaactcaacatagagggcttttagatttttcttcaaaaaaatttcgagaaaagtattcttttaccctccaaacagttaacagctcttagtttctccaaatatgctctttgatttacttatttttaattaaagatggtaatttattgaacaatgaaatccgtaatatattgatttaaggacaaaagtgaagttttagaattataaaagtacttaaatattatatattttccatttcataattgttttcctttctctgtggctttaaagtttttgactattttacaatgttaatcactaggtaacttgccatatttctggttctatattaagttctatcctttataatgctgttattataaagctggtttttagcatttgtctgtagcaatagaaattttactaagtctctgttctcccagtaagttttttcttttctcagtaagtccctaagaaaacatttgtttgccactcttactattcccaatcttggattgttcgagctgaaaaaaaatttgatgagaaacaggaggatccttttctggtgaatataggttcctgctttaagaatgtggaaatccattgctttatataactaatatacacacagattaattaaaattgtgagaaataattcacacatgacaagtaggtaacatgcatgagttttgaatttttttaaaaacccaactgtttgacaaaatatagaacccaaattggtactttcttagaccagtgtaacctcacacctcagttttgcttttccaaccctgacttgaaaggcatatttgtatctttttattagtgatagtgaagctgtgacactaaccttttatacaaaagagtaaagaaagaaaaactacagcgattaagatgagaacagttctgcagttgttgaactagatcacagcattgtaggcagaataaaaaatgttcatatctgagaatattcctttcgccatcttttcccaaggccagacctcctggtggagcacagttaaaagtaacattctgggcctttgtaatcggagggctgtgtctccagctggcagcctttgttttaatatataatgcaggactgtggaaaacagttggcatagaatattttcacctaaaaaagaaagaaaagacatacaaaactggattaattgcaaaaagagaatacagtaaaataccatataactggacaaagctagaagaacctttagaagatttgtctgaaaacagatttcaagagtgagcttttatacactgctcactaatttgcttgattactaccaactcttcttaaagttaacacgtttaaggtatttctggacttcctagccttttagcaagcttagaggaactagccattagctagtgatgtaaaaatattttggggactgatgcccttaaaggttatgcccttgaaagttcttaccttttctctagtgatattaaggaacgagtgggtagtgttctcagggtgaccagctgccctaaagtgcctgggattgagggtttccctggatgcgggactttccctggatacaaaacttttagcagagttttgtatatatgtggatttttctgataagtagcacatcagaggccttaaccactgcccaaaagcgattctccattgagagtacatatcttgaacttaagaaattcatttgctctgatttttaatcttgtaaagtttttgctaaactcaaaacaagtcccaggcacaccagaaggagctgaccaccttaggtgttcttgtgatttatccttacttccctatgttgtcatagttgcttctaaactcagctgcactatggctgtcaacatttctgatacttattgggatatgtgccatccagtcatttagtactttgaatggaacatgagatttataacacaggtaatagctgaaggtaccagtatggtggtgagactcacacttagtgatccagctaaggtaactgatgttataatggaacagagaagaggccaactagatagctaagttcttctgaacctatgtgtatatgtaagtacaaatcatgcgtccttatggggttaaacttaatctgaaatttacatttttcatagtaaaaggaaaccaattgttgcagatttcttttcttgtgaggaaatacatggcctttgatgctctggcgtctactgcatttcccagtctgttctgctcgagaagccagaatgtgttgttaacatttttccgtgaatgttgtgttaaaatgattaaatgcatcagccaatggcaagtgaaggaattgggtgtcctgatgcagactgagcagtttctctcaattgtagcctcatactcataaggtgcttaccagctagaacattgagcacgtgaggtgagattttttttctctgatggcattaactttgtaatgcaatatgatggatgcagaccctgttcttgtttccctctggaagtccttagtggctgcatccttggtgcactgtgatggagatattaaatgtgttctttgtgagctttcgttctatgattgtcaaaagtacgatgtggttccttttttatttttattaaacaatgagctgaggctttattacagctggttttcaagttaaaattgttgaatactgatgtctttctcccacctacaccaaatattttagtctatttaaagtacaaaaaaagttctgcttaagaaaacattgcttacatgtcctgtgatttctggtcaatttttatatatatttgtgtgcatcatctgtatgtgctttcactttttaccttgtttgctcttacctgtgttaacagccctgtcaccgttgaaaggtggacagttttcctagcattaaaagaaagccatttgagttgtttaccatgtta

SEQ ID NO: 30 - DNA, 6256 bp, Homo sapiens
  ref <308>: DATABASE ACCESSION NUMBER: GenBank Accession No.: NM_005902.3
gcggccgccgcctccgccccgcgttcggggccttcccgaccctgcactgctgccgtccgcccgcccggccgctcttctcttcgccgtgggagccgctccgggcgcagggccgcgcgccgagccccgcaggctgcagcgccgcggcccggcccggcgccccggcaacttcgccgagagttgaggcgaagtttgggcgaccgcggcaggccccggccgagctcccctctgcgcccccggcgtcccgtcgagcccagccccgccgggggcgctcctcgccgcccgcgcgccctccccagccatgtcgtccatcctgcctttcactcccccgatcgtgaagcgcctgctgggctggaagaagggcgagcagaacgggcaggaggagaaatggtgcgagaaggcggtcaagagcctggtcaagaaactcaagaagacggggcagctggacgagctggagaaggccatcaccacgcagaacgtcaacaccaagtgcatcaccatccccaggtccctggatggccggttgcaggtgtcccatcggaaggggctccctcatgtcatctactgccgcctgtggcgatggccagacctgcacagccaccacgagctacgggccatggagctgtgtgagttcgccttcaatatgaagaaggacgaggtctgcgtgaatccctaccactaccagagagtagagacaccagttctacctcctgtgttggtgccacgccacacagagatcccggccgagttccccccactggacgactacagccattccatccccgaaaacactaacttccccgcaggcatcgagccccagagcaatattccagagaccccaccccctggctacctgagtgaagatggagaaaccagtgaccaccagatgaaccacagcatggacgcaggttctccaaacctatccccgaatccgatgtccccagcacataataacttggacctgcagccagttacctactgcgagccggccttctggtgctccatctcctactacgagctgaaccagcgcgtcggggagacattccacgcctcgcagccatccatgactgtggatggcttcaccgacccctccaattcggagcgcttctgcctagggctgctctccaatgtcaacaggaatgcagcagtggagctgacacggagacacatcggaagaggcgtgcggctctactacatcggaggggaggtcttcgcagagtgcctcagtgacagcgctatttttgtccagtctcccaactgtaaccagcgctatggctggcacccggccaccgtctgcaagatcccaccaggatgcaacctgaagatcttcaacaaccaggagttcgctgccctcctggcccagtcggtcaaccagggctttgaggctgtctaccagttgacccgaatgtgcaccatccgcatgagcttcgtcaaaggctggggagcggagtacaggagacagactgtgaccagtaccccctgctggattgagctgcacctgaatgggcctttgcagtggcttgacaaggtcctcacccagatgggctccccaagcatccgctgttccagtgtgtcttagagacatcaagtatggtaggggagggcaggcttggggaaaatggccatgcaggaggtggagaaaattggaactctactcaacccattgttgtcaaggaagaagaaatctttctccctcaactgaaggggtgcacccacctgttttctgaaacacacgagcaaacccagaggtggatgttatgaacagctgtgtctgccaaacacatttaccctttggccccactttgaagggcaagaaatggcgtctgctctggtggcttaagtgagcagaacaggtagtattacaccaccggccccctccccccagactctttttttgagtgacagctttctgggatgtcacagtccaaccagaaacacccctctgtctaggactgcagtgtggagttcaccttggaagggcgttctaggtaggaagagcccgcagggccatgcagacctcatgcccagctctctgacgcttgtgacagtgcctcttccagtgaacattcccagcccagccccgccccgccccgccccaccactccagcagaccttgccccttgtgagctggatagacttgggatggggagggagggagttttgtctgtctccctcccctctcagaacatactgattgggaggtgcgtgttcagcagaacctgcacacaggacagcgggaaaaatcgatgagcgccacctctttaaaaactcacttacgtttgtcctttttcactttgaaaagttggaaggatctgctgaggcccagtgcatatgcaatgtatagtgtctattatcacattaatctcaaagagattcgaatgacggtaagtgttctcatgaagcaggaggcccttgtcgtgggatggcatttggtctcaggcagcaccacactgggtgcgtctccagtcatctgtaagagcttgctccagattctgatgcatacggctatattggtttatgtagtcagttgcattcattaaatcaactttatcatatgctcttttaaatgtttggtttatatattttctttaaaaatcctgggctggcacattgactgggaaacctgagtgagacccagcaactgcttctctcccttctctctcctgaggtgaagcttttccaggttttgttgaagagatacctgccagcacttctgcaagctgaaatttacagaagcaaattcaccagaagggaaacatctcaggccaacataggcaaatgaaaagggctattaaaatatttttacacctttgaaaattgcaggcttggtacaaagaggtctgtcatcttccccctgggatataagatgatctagctcccggtagaggatcaccggtgacaactatagcagttgtattgtgtaacaagtactgctcccagcagcaattagggagaaaactagtctaaattatttcaactggaaaaaagaaaaaagagtcctcttcttttcccagccttttgcagaacacagtagacagaactgccaccttcaattggtactttattctttgctgctgtttttgtataaaatgacctatcccacgtttttgcatgaatttatagcaggaaaaatcaagggatttcctatggaagtcctgctttattccaggtgaagggaaggaagtgtatatacttttggcaagtcatacagctcaaatgtgatgagatttctgatgttagagggagatggagaggcttcctgatgcctcatctgcagggtcctgtgcctctgaagttctagccatgaggtttccaggtaggacagctgctccccaagcctcctgaggacacaggaagagacggaaggagcaccttgacagacttgtgtgagtcttctcgaaggagggttgactcagaacccagagacaatacaaaacccctcacttcctctgagagggccaaatgctgtgagtctgaagtatgtgcctggtgtgaaatgatctatggcctgtttcttacacaggaagccccctgaacctcctgtacatgtgttcatgttcccagccagctctgagacccaggaaccaaatattccattttggcttctgctagagcagtcatggttcctctcctaaaagccatgggcagcagtttccgagggcctgcatgatccacctgctgcacgatcctatgagggcttcctgtggcacacagccctctgggtgcttgggaactagcttcaggcacagcctgattctggtgatccagtgatctatggaagtcgtgtcttactccaggtgaagggggaaaaaaaaagcctatactttggcaggttatgaactttgaatgtgatgaaatgacacgtttggctgcatttggatggtgtcttagaaccctcattgctcagacctgaaggctacttctaggagcatgaagtttgagttttgtgtttttccaaaggatacttccttggccctttttctttattgactagaccaccagaggaggatgtgtgggattgtaggcaaacccacctgtggcatcactgaaaataaatttgatcatacctaagaggttaggaaatggtgccattcccaccttagagtgctacataggtgctttgggcgtatgtaacattagtgtccttccttgaagccacaagctagttttcttagttttaaaatcctgttgtatgaatggcatttgtatattaaaacacttttttaaaggacagttgaaaagggcaagaggaaaccagggcagttctagaggagtgctggtgactggatagcagttttaagtggcgttcacctagtcaacacgaccgcgtgtgttgcccctgccctgggctccccgccatgacatcttcaccttgcagcttgtgctgagactgacccaagtgcagctagcactgggacacagatccttgtcttcagcaccttccaaggagccaacttttattccctttcctctctcccctccccacctcgcttcttcccaatttagtaacttagatgcttccagcacatacgtaggtagctaccccagccggtttggattacaggcctgtgctggaacatcatctcagttggccaccttcctggcaggctgtagacctgacattttgagacaagcctagagtcaggagcagggactttgactcttaggaagagcacacatgagggcaaggctgctggcagacgtctccattgtccttatgttgtctgtgttgtatttttttttttttattgaccatggtgattatttttttaaaccatcgttaatatactgaagtgagctatagcacatatcatgtgcttagtttgtttatttttctccatctccccttggcttcctagagtttggacatattccaggctaaatgcttttactcaagactacagaaaggtttgaagtagtgtgtgcatggcatgcacgtatgtaagtaatctggggaagaagcaaagatctgtttcattcttagcctcaggcctcatgagggtctccacagggccggagctcaggttacaccactccttcgtccttacaggagatgtagggagaagaatctgcaggctgcttgtaggactgttcaccaagggggataccagcagcaagagagtgcacccgtttagccctggaccctgtttcttactgtgtgacttggctagagttgggagttcccccaaaataaacgtgtccccattttaccagaaccaaacctcaacacagcgaagctgtactgtctttgtgtggcaaagatgttcccttgtaggcccctttcaggtaaccgtcttcacaatgtattttcatcacagtttaaggagcatcagccgcttctcaagtgggtagggaaagcagaaaaacgtacgcaagaggacatggatccaaaatgatgatgaagcatctcccatggggaggtgatggtggggagatgatgggctaaacaggcaacttttcaaaaacacagctatcatagaaaagaaacttgcctcatgtaaactggattgagaaattctcagtgattctgcaatggatttttttttaatgcagaagtaatgtatactctagtattctggtgtttttatatttatgtaataatttcttaaaaccattcagacagataactatttaattttttttaagaaagttggaaaggtctctcctcccaaggacagtggctggaagagttggggcacagccagttctgaatgttggtggagggtgtagtggctttttggctcagcatccagaaacaccaaaccaggctggctaaacaagtggccgcgtgtaaaaacagacagctctgagtcaaatctgggcccttccacaagggtcctctgaaccaagccccactcccttgctaggggtgaaagcattacagagagatggagccatctatccaagaagccttcactcaccttcactgctgctgttgcaactcggctgttctggactctgatgtgtgtggagggatggggaatagaacattgactgtgttgattaccttcactattcggccagcctgaccttttaataactttgtaaaaagcatgtatgtatttatagtgttttagatttttctaacttttatatcttaaaagcagagcacctgtttaagcattgtacccctattgttaaagatttgtgtcctctcattccctctcttcctcttgtaagtgcccttctaataaacttttcatggaaaagctcctgtgccaggagctcagtctgaaaaaaaaaaaaaaaaaaaaaaaaa

SEQ ID NO: 31 - DNA, 1335 bp, Homo sapiens
  ref <308>: DATABASE ACCESSION NUMBER: GenBank Accession No.: NM_001485.2
gacttttcgcctctcgctggcctctaccgagcgcgtctatgagcgcagcgttcccgccgtcgctgatgatgatgcagcgcccgctggggagtagcaccgccttcagcatagactcgctgatcggcagcccgccgcagcccagccccggccatttcgtctacaccggctaccccatgttcatgccctaccggccggtagtgctgccgccgccgccgccgccgccgcccgcgctgccccaggccgcgctgcagccagcgctgccgcccgcacaccctcaccaccagatccccagcctgcccacaggcttctgctccagcctggcgcagggcatggcgctcacctctacgctcatggccacgctccccggcggcttctccgcgtcgccccagcaccaggaggcggcagcggcccgcaagttcgcgccgcagccgctgcccggcggcggtaacttcgacaaggcggaggcgctgcaggctgacgcggaggacggcaaaggcttcctggccaaagagggctcgctgctcgccttctccgcggccgagacggtgcaggcttcgctcgtcggggctgtccgagggcaagggaaagacgagtcaaaggtggaagacgacccgaagggcaaggaggagagcttctcgctggagagcgatgtggactacagctcggatgacaatctgactggccaggcagctcacaaggaggaagacccgggccacgcgctggaggagaccccgccgagcagcggcgccgcgggcagcaccacgtctacgggcaagaaccggcggcggcggactgccttcaccagcgagcagctgctggagctagagaaggagttccactgcaaaaagtacctctccttgaccgagcgctcgcagatcgcccacgccctcaaactcagcgaggtgcaggtgaaaatctggttccagaaccgacgggccaagtggaaacgggtgaaggcaggcaatgccaattccaagacaggggagccctcccggaaccctaagatcgtcgtccccatccctgtccacgtcagcaggttcgctatcagaagtcagcatcagcagctagaacaggcccggccctgagggtccagaagggccagggcctggcacccacctggagaagcccccgcacccgagggaacccatggtggactccactgtgtttgaagcaacaaagtcacagcccagctgtggccatcccaagcaaattgagaatatattcactaaatgggcttaaaagactgcttttgaaggggcttacagccacaccagaagacacgctaaatatttattatactatcctactttgtacataaatatctctatagactgg

SEQ ID NO: 32 - DNA, 2518 bp, Homo sapiens
  ref <308>: DATABASE ACCESSION NUMBER: GenBank Accession No.: NM_003106.2
ctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaaaaaaaaaaaaaaaaa

SEQ ID NO: 33 - DNA, 1912 bp, Homo sapiens
  ref <308>: DATABASE ACCESSION NUMBER: GenBank Accession No.: NM_001008540.1
ttttttttcttccctctagtgggcggggcagaggagttagccaagatgtgactttgaaaccctcagcgtctcagtgcccttttgttctaaacaaagaattttgtaattggttctaccaaagaaggatataatgaagtcactatgggaaaagatggggaggagagttgtaggattctacattaattctcttgtgcccttagcccactacttcagaatttcctgaagaaagcaagcctgaattggttttttaaattgctttaaaaattttttttaactgggttaatgcttgctgaattggaagtgaatgtccattcctttgcctcttttgcagatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 34 - DNA, 4353 bp, Homo sapiens
  ref <308>: DATABASE ACCESSION NUMBER: GenBank Accession No.: NM_006892.3
acccactcccgctgccccgtccggcccgcgccgcttcctcgcagcagctgctcccggctccgcggccgcagcccgcgtggacgctccgagcgccccccgacggacgggaccggctccctggcggtcgggcgagcgggcggcaacgctgcccggccggcagcgctggggttaagtggcccaagtaaacctagctcggcgatcggcgccggagattcgcgagcccagcgccctgcacggccgccagccggcctcccgccagccagccccgacccgcggctccgccgcccagccgcgccccagccagccctgcggcaggaaagcatgaagggagacaccaggcatctcaatggagaggaggacgccggcgggagggaagactcgatcctcgtcaacggggcctgcagcgaccagtcctccgactcgcccccaatcctggaggctatccgcaccccggagatcagaggccgaagatcaagctcgcgactctccaagagggaggtgtccagtctgctaagctacacacaggacttgacaggcgatggcgacggggaagatggggatggctctgacaccccagtcatgccaaagctcttccgggaaaccaggactcgttcagaaagcccagctgtccgaactcgaaataacaacagtgtctccagccgggagaggcacaggccttccccacgttccacccgaggccggcagggccgcaaccatgtggacgagtcccccgtggagttcccggctaccaggtccctgagacggcgggcaacagcatcggcaggaacgccatggccgtcccctcccagctcttaccttaccatcgacctcacagacgacacagaggacacacatgggacgccccagagcagcagtaccccctacgcccgcctagcccaggacagccagcaggggggcatggagtccccgcaggtggaggcagacagtggagatggagacagttcagagtatcaggatgggaaggagtttggaataggggacctcgtgtggggaaagatcaagggcttctcctggtggcccgccatggtggtgtcttggaaggccacctccaagcgacaggctatgtctggcatgcggtgggtccagtggtttggcgatggcaagttctccgaggtctctgcagacaaactggtggcactggggctgttcagccagcactttaatttggccaccttcaataagctcgtctcctatcgaaaagccatgtaccatgctctggagaaagctagggtgcgagctggcaagaccttccccagcagccctggagactcattggaggaccagctgaagcccatgttggagtgggcccacgggggcttcaagcccactgggatcgagggcctcaaacccaacaacacgcaaccagtggttaataagtcgaaggtgcgtcgtgcaggcagtaggaaattagaatcaaggaaatacgagaacaagactcgaagacgcacagctgacgactcagccacctctgactactgccccgcacccaagcgcctcaagacaaattgctataacaacggcaaagaccgaggggatgaagatcagagccgagaacaaatggcttcagatgttgccaacaacaagagcagcctggaagatggctgtttgtcttgtggcaggaaaaaccccgtgtccttccaccctctctttgagggggggctctgtcagacatgccgggatcgcttccttgagctgttttacatgtatgatgacgatggctatcagtcttactgcactgtgtgctgcgagggccgagagctgctgctttgcagcaacacgagctgctgccggtgtttctgtgtggagtgcctggaggtgctggtgggcacaggcacagcggccgaggccaagcttcaggagccctggagctgttacatgtgtctcccgcagcgctgtcatggcgtcctgcggcgccggaaggactggaacgtgcgcctgcaggccttcttcaccagtgacacggggcttgaatatgaagcccccaagctgtaccctgccattcccgcagcccgaaggcggcccattcgagtcctgtcattgtttgatggcatcgcgacaggctacctagtcctcaaagagttgggcataaaggtaggaaagtacgtcgcttctgaagtgtgtgaggagtccattgctgttggaaccgtgaagcacgaggggaatatcaaatacgtgaacgacgtgaggaacatcacaaagaaaaatattgaagaatggggcccatttgacttggtgattggcggaagcccatgcaacgatctctcaaatgtgaatccagccaggaaaggcctgtatgagggtacaggccggctcttcttcgaattttaccacctgctgaattactcacgccccaaggagggtgatgaccggccgttcttctggatgtttgagaatgttgtagccatgaaggttggcgacaagagggacatctcacggttcctggagtgtaatccagtgatgattgatgccatcaaagtttctgctgctcacagggcccgatacttctggggcaacctacccgggatgaacaggcccgtgatagcatcaaagaatgataaactcgagctgcaggactgcttggaatacaataggatagccaagttaaagaaagtacagacaataaccaccaagtcgaactcgatcaaacaggggaaaaaccaacttttccctgttgtcatgaatggcaaagaagatgttttgtggtgcactgagctcgaaaggatctttggctttcctgtgcactacacagacgtgtccaacatgggccgtggtgcccgccagaagctgctgggaaggtcctggagcgtgcctgtcatccgacacctcttcgcccctctgaaggactactttgcatgtgaatagttccagccaggccccaagcccactggggtgtgtggcagagccaggacccaggaggtgtgattcctgaaggcatccccaggccctgctcttcctcagctgtgtgggtcataccgtgtacctcagttccctcttgctcagtgggggcagagccacctgactcttgcaggggtagcctgaggtgccgcctccttgtgcacaaatcagacctggctgcttggagcagcctaacacggtgctcattttttcttctcctaaaactttaaaacttgaagtaggtagcaacgtggctttttttttttcccttcctgggtctaccactcagagaaacaatggctaagataccaaaaccacagtgccgacagctctccaatactcaggttaatgctgaaaaatcatccaagacagttattgcaagagtttaatttttgaaaactggctactgctctgtgtttacagacgtgtgcagttgtaggcatgtagctacaggacatttttaagggcccaggatcgttttttcccagggcaagcagaagagaaaatgttgtatatgtcttttacccggcacattccccttgcctaaatacaagggctggagtctgcacgggacctattagagtattttccacaatgatgatgatttcagcagggatgacgtcatcatcacattcagggctattttttcccccacaaacccaagggcaggggccactcttagctaaatccctccccgtgactgcaatagaaccctctggggagctcaggaaggggtgtgctgagttctataatataagctgccatatattttgtagacaagtatggctcctccatatctccctcttccctaggagaggagtgtgaagcaaggagcttagataagacaccccctcaaacccattccctctccaggagacctaccctccacaggcacaggtccccagatgagaagtctgctaccctcatttctcatctttttactaaactcagaggcagtgacagcagtcagggacagacatacatttctcataccttccccacatctgagagatgacagggaaaactgcaaagctcggtgctccctttggagattttttaatccttttttattccataagaagtcgtttttagggagaacgggaattcagacaagctgcatttcagaaatgctgtcataatggtttttaacaccttttactcttcttactggtgctattttgtagaataaggaacaacgttgacaagttttgtggggctttttatacactttttaaaatctcaaacttctatttttatgtttaacgttttcattaaaattttttttgtaactggagccacgacgtaacaaatatggggaaaaaactgtgccttgtttcaacagtttttgctaatttttaggctgaaagatgacggatgcctagagtttaccttatgtttaattaaaatcagtatttgtctaaaaaaaaaaaaaaaaaa

SEQ ID NO: 35 - DNA, 4815 bp, Homo sapiens
  ref <308>: DATABASE ACCESSION NUMBER: GenBank Accession No.: NM_004360.3
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgctggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgccttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaatttttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaaccat

SEQ ID NO: 36 - DNA, 1321 bp, Homo sapiens
  ref <308>: DATABASE ACCESSION NUMBER: GenBank Accession No.: NM_001769.3
cttttcccggcacatgcgcaccgcagcgggtcgcgcgccctaaggagtggcactttttaaaagtgcagccggagaccagcctacagccgcctgcatctgtatccagcgccaggtcccgccagtcccagctgcgcgcgccccccagtcccgcacccgttcggcccaggctaagttagccctcaccatgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctagagtcagcttacatccctgagcaggaaagtttacccatgaagattggtgggattttttgtttgtttgttttgttttgtttgttgtttgttgtttgtttttttgccactaattttagtattcattctgcattgctagataaaagctgaagttactttatgtttgtcttttaatgcttcattcaatattgacatttgtagttgagcggggggtttggtttgctttggtttatattttttcagttgtttgtttttgcttgttatattaagcagaaatcctgcaatgaaaggtactatatttgctagactctagacaagatattgtacataaaagaatttttttgtctttaaatagatacaaatgtctatcaactttaatcaagttgtaacttatattgaagacaatttgatacataataaaaaattatgacaatgtcctggactggtaaaaaaa

SEQ ID NO: 37 - DNA, 5246 bp, Homo sapiens
  ref <308>: DATABASE ACCESSION NUMBER: GenBank Accession No.: NM_080759.4
atctttgatcaatgtacttgccagggagagcccaagtccttcaaacctcctccttttcaccttcatccttaactttgtgctagagcgagacccacacaacaacagccgaccctccccgccccacccccacccccaaaccagccctcgatcccagcccccggagaggactcgcatttcgacttgcgggacacttttgtgcgttcctctccagagcgcctctcgtgctcgcccctcttgcgctcgctctttattaccttcacctccttttctcccccttctctccctttctccttctcgttctctcccggagttgttgttgcccccctcgctccttctccccccttttttccccttcccctcccgggggtgtgtggcaacttttcctctcgcttctcctccgtctgtttccccttatatgtgaccatggcagtgccggcggctttgatccctccgacccagctggtcccccctcaacccccaatctccacgtctgcttcctcctctggcaccaccacctccacctcttcggcgacttcgtctccggctccttccatcggacccccggcgtcctctgggccaactctgttccgcccggagcccatcgcttcggcggcggcggcggcggccacagtcacctctaccggcggcggcggcggcggcggcggcggcggcagcggaggcggcggcggcagcagcggcaacggaggcggcggtggcggcggcggcggtggcagcaactgcaaccccaacctggcggccgcgagcaacggcagcggcggcggcggcggcggcatcagcgctggcggcggcgtcgcttccagcacccccatcaacgccagcaccggcagcagcagcagcagcagtagcagcagcagcagcagcagcagtagtagcagcagcagcagtagcagcagcagctgcggccccctccccgggaaacccgtgtactcaaccccgtccccagtggaaaacacccctcagaataatgagtgcaaaatggtggatctgaggggggccaaagtggcttccttcacggtggagggctgcgagctgatctgcctgccccaggctttcgacctgttcctgaagcacttggtggggggcttgcatacggtctacaccaagctgaagcggctggagatcacgccggtggtgtgcaatgtggaacaagttcgcatcctgaggggactgggcgccatccagccaggagtgaaccgctgcaaactcatctccaggaaggacttcgagaccctctacaatgactgcaccaacgcaagttctagacctggaaggcctcctaagaggactcaaagtgtcacctccccagagaactctcacatcatgccgcattctgtccctggtctcatgtctcctgggataattccaccaacaggtctgacagcagccgctgcagcagctgctgctgctaccaatgcagctattgctgaagcaatgaaggtgaaaaaaatcaaattagaagccatgagcaactatcatgccagtaataaccaacatggagcagactctgaaaacggggacatgaattcaagtgtcggactggaacttccttttatgatgatgccccaccctctaattcctgtcagcctacctccagcatctgtcaccatggcaatgagccagatgaaccacctcagcaccattgcaaatatggcagcagcagcacaagttcagagtcccccatccagagttgagacatcagttattaaggagcgtgttcctgatagcccctcacctgccccctctctggaggaggggagaaggcctggcagtcacccatcatcacatcgcagcagcagcgtgtccagctcccctgctcggactgagagctcttctgacagaatcccggtccatcagaatgggttgtccatgaaccagatgctgatgggcttatcaccaaatgtacttcctgggcccaaagagggagatttggccggtcatgacatgggacatgagtcaaaaaggatgcatattgaaaaagatgagaccccgctttctacaccaaccgcaagagacagccttgacaaactctctctaactgggcatggacaaccactgcctccaggttttccatctccttttctgtttcctgatggactgtcttccatcgagactcttctgactaacatacaggggctgttgaaagttgccatagataatgccagagctcaagagaaacaggtccaactggaaaaaactgagctgaagatggattttttaagggaaagagaactaagggaaacacttgagaagcagttggctatggaacaaaagaatagagccatagttcaaaagaggctaaagaaggagaagaaggcaaagagaaaattgcaggaagcacttgagtttgagacgaaacggcgtgaacaagcagaacagacgctaaaacaggcagcttcaacagatagtctcagggtcttaaatgactctctgaccccagagatagaggctgaccgcagtggcggcagaacagatgctgaaaggacaatacaagatggaagactgtatttgaaaactactgtcatgtactgaatctttcctgttgaagaaatccatgttatagaaaagaactttgcagtcagacattcgtcatgggaaagttcagaaaaaaataaagtccttttaagggaacttcctgaattttgtgtattaatgttctttaaaagtttaagtattctacaaaaaaaaaaaaagttttctccattgattttcacctgtggttcataccagagacctgagaatgtttgtaaatgtacaagtatcaaagttcttacagttaattactgcaacttgctgctggacaattgtatacagagttaaaggcaggtctgaataagacctagctttgtttttttctaatggaatgaaccattttcctcttctgaaaattctgtatctgagcacatcaagagactcttgtagcagtggttacccagacttacagaattatgtcctccagaaaccagcaagaacacttggaatgaacgaatgaacttgtagggggcatagaggattcttgaaaaaaaaaaatgcaagagtgattttctgttacattcaatttcaaactctctaattgtgggttttctcctgaagaattttttttcacatactttccaaaagaccaacaaatggatgttgacaacaacccaatgaaataacattttgcatatctgaaaagaagcattgaatataagccaaaagctttcactgaaggtttttttttcttaaaaataaaaaaaaatatataagtgtaacatgttttcattccaaactggtagtggtatatagaattaaagataataatgttgcttcttattcaaactgttggtcatatgtacagtatataaacataaaacacacaaggaaggtattatgtatgcagtagtatactagagtttaggaaaatgaaaattttagaaaatatgttttgtcaccctgttggtcagaaagatgtctttctggttttaacgcatgcaggcatgtaaatatttgtctggagtcacagtattaatgaatgagatcttaagcatctggtgacatcagaactctgtgtcagccacttttatttgtatattgaaccctagctagtgccccaagctgcactattgggaatggattgtggctgaacagcaaatcaaaacaccagaaatatttttatatgttaacgtcatattatgttaatgttgctgaaaacaaaacctaacaaaccttgatgtaccagtccaataccatgtagcgctgagtgataaagttaaaatgtgctgtgcttcccacccttgtcagagggaagggtggctatgtgttattttcactgtctttttgaaagttacagtatgtgttttcactttcgtgcagataactggaagtaaagcggcaaacagtgcttattacatgctaaagttaccttctctttgttttttgcatatctggaattacacctttaaagactgatatgaatcagtacggtcactatacattttatgatttttctgtcatcttaaaattgtatgatcgtaacattatttattaccacaaaacagcaaaatcttcaatgtctaagaaaactagcttaaaatgtttaaatatagttctgattgggtattaattacttgattaagaaaaaattaacattatagatactctggcattacgcttctataccttttaggtcttccttgcaatactggaacataattcttttgtgtagctcactattagccagctaagttcatctttttaataccataaaaaggttatatgtacagttcctattttagcttgcttacaaagggagcattatttttatttaaagtattgctagtaaatgatttgtagaaacttggttttctaagcatagttcttccataaccaccttttgttgtttgagcacaagggattcttttcctagttctatgtgtttgtttccctatatgcagtctttaaaggattacaacacttaaaattgaatggacttgtgtcaagctttttgcatcatacattttttgaaagatttttaaaaaagcctacaacttacatatgtagtagaatcagccattgctctgctcctggcatagagtcacctgtttgttatgtggattaaatagttttaaaatacatatttgaagacctttgagaatgctttagtgtttgatttgaaataaaaggaaattttagcaaggattaaagaaaaaagctatcagctgtatgttaagagagactcttactaacatgttgtaaatattacaattcatgaaatgttattgtaagtctgtaacttaattttttccctgttttagttatacaggttggtttggaaatttgtgttttggcataaacaagtaaaatgtgcccattttatggtttccatgcttttgtaatcctaaaaatattaatgtctagttgttctatattataaccacatttgcgctctatgcaagcccttggaacagaacatactcatcttcatgtaggacctatgaaaattgtctatttttatctatatatttaaagttttctaaaaatgataaaaggttattacgaattttgttgtacaaaatctgtacaaaaatctgtttttacatcataatgcaagaattggaaatttttctatggtagcctagttatttgagcctggtttcaatgtgagaaccacgtttactgttattgtatttaattttcttttccttttcaacaatctcctaataaaactgtctgaaatctcaaaaaaa

SEQ ID NO: 38 - DNA, 2908 bp, Homo sapiens
  ref <308>: DATABASE ACCESSION NUMBER: GenBank Accession No.: NM_004098.3
cgggcgccgcaggagcgagtgagctgggagcgaggggcgaaggcgcggagaagcccggccgcccggtgggcggcagaaggctcagccgaggcggcggcgccgactccgttccactctcggcccggatccaggcctccgggttcccaggcgctcacctccctctgacgcactttaaagagtctccccccttccacctcagggcgagtaatagcgaccaatcatcaagccatttaccaggcttcggaggaagctgtttatgtgatccccgcactaattaggctcatgaactaacaaatcgtttgcacaacttgtgaagaagcgaacacttccatggattgtccttggacttagggcgccctgcccgccttttgcagaggagaaaaaacttttttttttttttgcctcccccgagaactttccccccttctcctccctgcctctaactccgatccccccacgccatctcgccaaaaaaaaaaaaaaaaaaaaaaaagaaaaaaaaagaaaaaaaaagaaaaaaaattaccccaatccacgcctgcaaattcttctggaaggattttcccccctctcttcaggttgggcgcgtttggtgcaagattctcgggatcctcggctttgcctctccctctccctcccccctcctttcctttttcctttcctttcctttctttcttcctttccttccccccacccccacccccaccccaaacaaacgagtccccaattctcgtccgtcctcgccgcgggcagcgggcggcggaggcagcgtgcggcggtcgccaggagctgggagcccagggcgcccgctcctcggcgcagcatgttccagccggcgcccaagcgctgcttcaccatcgagtcgctggtggccaaggacagtcccctgcccgcctcgcgctccgaggaccccatccgtcccgcggcactcagctacgctaactccagccccataaatccgttcctcaacggcttccactcggccgccgccgccgccgccggtaggggcgtctactccaacccggacttggtgttcgccgaggcggtctcgcacccgcccaaccccgccgtgccagtgcacccggtgccgccgccgcacgccctggccgcccaccccctaccctcctcgcactcgccacaccccctattcgcctcgcagcagcgggatccgtccaccttctacccctggctcatccaccgctaccgatatctgggtcatcgcttccaagggaacgacactagccccgagagtttccttttgcacaacgcgctggcccgaaagcccaagcggatccgaaccgccttctccccgtcccagcttctaaggctggaacacgcctttgagaagaatcactacgtggtgggcgccgaaaggaagcagctggcacacagcctcagcctcacggaaactcaggtaaaagtatggtttcagaaccgaagaacaaagttcaaaaggcagaagctggaggaagaaggctcagattcgcaacaaaagaaaaaagggacgcaccatattaaccggtggagaatcgccaccaagcaggcgagtccggaggaaatagacgtgacctcagatgattaaaaacataaacctaaccccacagaaacggacaacatggagcaaaagagacagggagaggtggagaaggaaaaaaccctacaaaacaaaaacaaaccgcatacacgttcaccgagaaagggagagggaatcggagggagcagcggaatgcggcgaagactctggacagcgagggcacagggtcccaaaccgaggccgcgccaagatggcagaggatggaggctccttcatcaacaagcgaccctcgtctaaagaggcagctgagtgagagacacagagagaaggagaaagagggagggagagagagaaagagagagaaagagagagagagagagagagagagaaagctgaacgtgcactctgacaaggggagctgtcaatcaaacaccaaaccggggagacaagatgattggcaggtattccgtttatcacagtccacttaaaaaatgatgatgatgataaaaaccacgacccaaccaggcacaggacttttttgttttttgcacttcgctgtgtttcccccccatctttaaaaataattagtaataaaaaacaaaaattccatatctagccccatcccacacctgtttcaaatccttgaaatgcatgtagcagttgttgggcgaatggtgtttaaagaccgaaaatgaattgtaattttcttttccttttaaagacaggttctgtgtgctttttattttgattttttttcccaagaaatgtgcagtctgtaaacactttttgataccttctgatgtcaaagtgattgtgcaagctaaatgaagtaggctcagcgatagtggtcctcttacagagaaacggggagcaggacgacgggggggctgggggtggcgggggagggtgcccacaaaaagaatcaggacttgtactgggaaaaaaacccctaaattaattatatttcttggacattccctttcctaacatcctgaggcttaaaaccctgatgcaaacttctcctttcagtggttggagaaattggccgagttcaaccattcactgcaatgcctattccaaactttaaatctatctattgcaaaacctgaaggactgtagttagcggggatgatgttaagtgtggccaagcgcacggcggcaagttttcaagcactgagtttctattccaagatcatagacttactaaagagagtgacaaatgcttccttaatgtcttctataccagaatgtaaatatttttgtgttttgtgttaatttgttagaattctaacacactatatacttccaagaagtatgtcaatgtcaatattttgtcaataaagatttatcaatatgccctcaaaaaaaaaaaaa

SEQ ID NO: 39 - DNA, 1005 bp, Homo sapiens
  ref <308>: DATABASE ACCESSION NUMBER: GenBank Accession No.: NM_001446.3
gaggattgggaggaactcgacctactccgctaacccagtggcctgagccaatcacaaagaggattggagcctcactcgagcgctccttcccttctcctctctctgtgacagcctcttggaaagagggacactggaggggtgtgtttgcaatttaaatcactggatttttgcccaccctctttccaaataagaaggcaggagctgcttgctgaggtgtaaagggtcttctgagctgcagtggcaattagaccagaagatccccgctcctgtctctaaagaggggaaagggcaaggatggtggaggctttctgtgctacctggaagctgaccaacagtcagaactttgatgagtacatgaaggctctaggcgtgggctttgccactaggcaggtgggaaatgtgaccaaaccaacggtaattatcagtcaagaaggagacaaagtggtcatcaggactctcagcacattcaagaacacggagattagtttccagctgggagaagagtttgatgaaaccactgcagatgatagaaactgtaagtctgttgttagcctggatggagacaaacttgttcacatacagaaatgggatggcaaagaaacaaattttgtaagagaaattaaggatggcaaaatggttatgacccttacttttggtgatgtggttgctgttcgccactatgagaaggcataaaaatgttcctggtcggggcttggaagagctcttcagtttttctgtttcctcaagtctcagtgctatcctattacaacatggctgatcattaattagaaggttatccttggtgtggaggtggaaaatggtgatttaaaaacttgttactccaagcaacttgcccaattttaatctgaaaatttatcatgttttataatttgaattaaagttttgtcccccccccccttttttttataaacaagtgaatacattttataatttcttttggaatgtaaatcaaatttgaataaaaatcttacacgtgaaaaaaaa

SEQ ID NO: 40 - DNA, 2379 bp, Homo sapiens
  ref <308>: DATABASE ACCESSION NUMBER: GenBank Accession No.: NM_002467.4
gacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaaaaaaaaaaaaaaa

SEQ ID NO: 41 - DNA, 2518 bp, Homo sapiens
  ref <308>: DATABASE ACCESSION NUMBER: GenBank. Accession No. NM_003181
tttgcttttgcttatttccgtccatttccctctctgcgcgcggaccttccttttccagatggtgagagccgcggggacacccgacgccggggcaggctgatccacgatcctgggtgtgcgtaacgccgcctggggctccgtgggcgagggacgtgtggggacaggtgcaccggaaactgccagactggagagttgaggcatcggaggcgcgagaacagcactactactgcggcgagacgagcgcggcgcatcccaaagcccggccaaatgcgctcgtccctgggaggggagggaggcgcgcctggagcggggacagtcttggtccgcgccctcctcccgggtctgtgccgggacccgggacccgggagccgtcgcaggtctcggtccaaggggccccttttctcggaagggcggcggccaagagcagggaaggtggatctcaggtagcgagtctgggcttcggggacggcggggaggggagccggacgggaggatgagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgcttatcagaacgaggagatcacagctcttaaaattaagtacaatccatttgcaaaagctttccttgatgcaaaggaaagaagtgatcacaaagagatgatggaggaacccggagacagccagcaacctgggtactcccaatgggggtggcttcttcctggaaccagcaccctgtgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatgtgaagcagcaaggcccaggtcccgaaagatgcagtgactttttgtcgtggcagccagtggtgactggattgacctactaggtacccagtggcagtctcaggttaagaaggaaatgcagcctcagtaacttccttttcaaagcagtggaggagcacacggcacctttccccagagccccagcatcccttgctcacacctgcagtagcggtgctgtcccaggtggcttacagatgaacccaactgtggagatgatgcagttggcccaacctcactgacggtgaaaaaatgtttgccagggtccagaaactttttttggtttatttctcatacagtgtattggcaactttggcacaccagaatttgtaaactccaccagtcctactttagtgagataaaaagcacactcttaatcttcttccttgttgctttcaagtagttagagttgagctgttaaggacagaataaaatcatagttgaggacagcaggttttagttgaattgaaaatttgactgctctgccccctagaatgtgtgtattttaagcatatgtagctaatctcttgtgttgttaaactataactgtttcatatttttcttttgacaaagtagccaaagacaatcagcagaaagcattttctgcaaaataaacgcaatatgcaaaatgtgattcgtccagttattagtgaagcccctccttttgtgagtatttactgtttattg

SEQ ID NO: 42 - DNA, 19 bp, Artificial Sequence
  shRNA1
agtaaagagtggagaaaga

SEQ ID NO: 43 - DNA, 19 bp, Artificial Sequence
  shRNA2
acgtgtaggttgagaacaa

SEQ ID NO: 44 - DNA, 19 bp, Artificial Sequence
  shRNA3
gagaaaggtcagaaagaca

(19) United States
(12) Patent Application Publication
(10) Pub. No.: US 2012/0101024 A1
(43) Pub. Date: Apr. 26, 2012

(54) Methods and Compositions for Differentiating Embryonic Stem Cells

(75) Inventors: Sally Temple Stern, Slingerlands, NY (US); Christopher A. Fasano, Glenmont, NY (US)

(73) Assignee: REGENERATIVE RESEARCH FOUNDATION, Rensselaer, NY (US)

(21) Appl. No.: 13/282,132

(22) Filed: Oct. 26, 2011

Related U.S. Application Data
(60) Provisional application No. 61/406,843, filed on Oct. 26, 2010.

(51) Int. Cl. A61K 38/02; A61P 35/00; A61K 31/7088; C12N 5/079; C12N 5/0735

(52) U.S. Cl. 514/1.1; 435/377; 435/366; 514/44 R

(57) ABSTRACT

Methods and compositions for differentiating mammalian stem and progenitor cells are provided. More particularly, methods and compositions for obtaining neural cells from human embryonic stem cells are provided.

### RELATED APPLICATIONS

The present application claims the benefit of U.S. Provisional Patent Application Ser. No. 61/406,843, filed Oct. 26, 2010, which is herein incorporated by reference in its entirety.

### REFERENCE TO SEQUENCE LISTING

Pursuant to 37 C.F.R. 1.821(c), a sequence listing is submitted herewith via EFS-Web as an ASCII compliant text file named “SequenceListing.txt” that was created on Oct. 25, 2011, and has a size of 163,532 bytes. The content of the aforementioned file named “SequenceListing.txt” is hereby incorporated by reference in its entirety.

### FIELD OF THE INVENTION

The present invention provides methods and compositions for differentiating mammalian stem cells. More particularly, the invention relates to methods and compositions for deriving neural cells from human ESCs.

### BACKGROUND OF THE INVENTION

Studying the development of the nervous system dates back to the early 20th century, when scientists used animal models such as newts and xenopus to perform simple experiments testing what molecules were critical for nervous system induction. These studies have continued using rodent models, where new technology has allowed for genetic studies leading to the discovery of important genes involved in this process. While all of these studies have been invaluable in pushing the field forward, little has been done in humans due to the difficulty of gaining access to early fetal tissue to assess how the human nervous system is specified, i.e., how neural cells are differentiated from stem and progenitor cells.

In recent years, however, human embryonic stem cells (hESCs) have offered a potential answer to this problem, allowing for an accessible and manipulatable cell platform to model early human neural development. One recent study used hESCs to show that a gene expressed during the early stages of development is very important for human but not mouse brain development. This study not only demonstrated the power of using hESCs as a model system for studying human neural development, but also demonstrated a key evolutionary difference between rodent and man in regard to critical regulators of brain development, driving home the idea that more studies using human cells need to be performed.

With the advent of human pluripotent cell technology, the generation of human neural cell types to study and treat neural disorders has become a realistic endeavor. However, while significant progress has been made in generating neural cell types, most protocols employ expensive cytokine cocktails at concentrations well beyond that of physiological relevance and/or drugs that tend to have off-target effects at the concentrations used. Additionally, drugs are rarely specific and therefore non-specific effects cannot be ruled out. Thus, in order to efficiently generate neural cells in a more defined and biologically relevant manner, improved methods and compositions for deriving neural stem and progenitor cells from stem cells, and particularly from human stem cells are needed.

### SUMMARY OF THE INVENTION

As follows from the Background Section, there is a clear need in the art to develop novel methods for obtaining neural cells, such as, e.g., neural stem cells (NSCs) and neural progenitor cells (NPCs), from stem and/or progenitor cells.

Thus, in one embodiment, a method for obtaining a neural cell is provided, the method comprising administering to a stem or progenitor cell an inhibitor of the Nodal/TGF beta signaling pathway or an inhibitor of the BMP signaling pathway. In one embodiment, the inhibitor is a pentraxin selected from the group consisting of NPTX1, NPTX2 and CRP. A pentraxin polypeptide can be administered to the stem cell or progenitor cell or an expression construct encoding the pentraxin polypeptide can be administered to the stem cell or progenitor cell such that the stem cell or progenitor cell expresses the pentraxin polypeptide encoded by the expression construct. In a preferred embodiment, the pentraxin is NPTX1.

In a preferred embodiment, an inhibitor binds to CRIPTO.

A method for treating a disease or condition associated with aberrant Nodal/TGF beta signaling or aberrant BMP signaling is also provided, the method comprising administering to a subject in need thereof an effective amount for treating said condition of a pentraxin, such as NPTX1. NPTX1 or other pentraxin can be administered as a polypeptide or as a construct encoding NPTX1 polypeptide. A disease or condition for treatment can be a member selected from the group consisting of cancer, heart disease, muscular dystrophy, stroke, blood aneurisms and vessel aneurisms.

Also provided is a pharmaceutical composition comprising a pentraxin polypeptide, such as NPTX1 NPTX2, NPTXR, or CRP, and a pharmaceutically acceptable carrier. Preferably, the pentraxin is NPTX1 or CRP. A method for treating a disease or condition associated with aberrant Nodal/TGF beta signaling or aberrant BMP signaling is also provided, the method comprising administering to a subject in need thereof a therapeutically effective amount of a pharmaceutical composition provided herein.

In one embodiment, a method for maintaining a mammalian stem or progenitor cell in an undifferentiated state is provided, the method comprising incubating the stem or progenitor cell in the presence of an inhibitor of NPTX1.

In another embodiment, a method for increasing differentiation of stem or progenitor cells toward a mesodermal lineage is provided, the method comprising administering to the stem or progenitor cells an inhibitor of the Nodal/TGF beta signaling pathway or an inhibitor of the BMP signaling pathway is provided, wherein the stem or progenitor cells are cultured in conditions appropriate for mesodermal differentiation. Preferably, the mesodermal lineage is selected from the group consisting of blood, heart, skeletal muscle, and smooth muscle. Also preferred is that the inhibitor is administered in an effective amount for increasing mesodermal differentiation such that the resulting population of cells consists of at least 60% cells of the mesodermal lineage. More preferably, the population of cells consists of at least 90% cells of the mesodermal lineage.

In any of the above embodiments, a stem cell can be selected from the group consisting of a skin stem cell, a spermatagonial stem cell, a hair follicle stem cell, a cancer stem cell, a bone marrow stem cell, a gut stem cell, a hematopoietic stem cell, an adipose stem cell, a mammalian embryonic stem cell (ESC), a retinal pigment epithelial stem cell, a mesenchymal stem cell, an epiblast stem cell, a renal stem cell, an amniotic stem cell, an umbilical blood stem cell, an endothelial stem cell, a neural crest stem cell, and an induced pluripotent stem cell (iPSC). A mammalian ESC can be a human ESC.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a line graph showing endogenous gene expression (mRNA) of NPTX1 and PAX6 on day (D) 1, 3, 5, 7 and 11 of neural induction in hESCs. Data is expressed as fold change over day 1.

FIG. 2A is a bar graph showing fold change in gene expression (mRNA) of NPTX1, SOX1 and PAX6 genes on day 7 of neural induction after NPTX1 knockdown with the indicated shRNA constructs (1, 2 ord 3) or control (no shRNA) and FIG. 2B is a line graph showing fold change (from day 0) in mRNA expression of the early neuroectodermal markers GBX2 and SOX2 on days 0, 2, 3, 5 and 7, relative to day 0, in hESCs transduced with NPTX1-specific shRNA3 (indicated by square symbols) that knocks down NPTX1 gene expression, or transduced with a control shRNA (indicated by circle symbols) that does not knock down NPTX1 gene expression.

FIG. 3 is a bar graph showing the number of neurospheres on day 7 of differentiation in hESCs treated with NPTX1 shRNA2 or control (empty vector).

FIGS. 4A-4C contains line graphs showing fold change in gene expression of NPTX1 (FIG. 4A), PAX6 (FIG. 4B) and SOX1 (FIG. 4C) at the indicated time points (days, “D”) in differentiating hESCs transduced with NPTX1 shRNA or in control hESCs (empty vector transduced cells)

FIGS. 5A and 5C are line graphs showing the fold change in mRNA expression of mesodermal marker brachyury (FIG. 5A) and endodermal marker SOX17, on days 0, 4, 5 and 8, and FIG. 5B is a bar graph showing the percentage of KDR+ mesodermal cells in the cell culture on day 4, in control cells (no shRNA treatment) or in cells treated with NPTX1 shRNA to knock down NPTX1 that were differentiated toward mesodermal (FIGS. 5A, 5B) or endodermal (FIG. 5C) lineages.

FIGS. 6A-6D are line graphs showing fold change in gene expression of NPTX1 (FIG. 6A), NANOG (FIG. 6B), PAX6 (FIG. 6C) and SOX1 (FIG. 6D) at the indicated time points (days, “D”) in control and NPTX1-overexpressing hESCs (“NPTX1over”) under conditions driving neural differentiation.

FIG. 7 is a bar graph showing the fold change relative to control in gene expression of SOX17, Brachyury and PAX6 in spontaneously differentiating control and NPTX1-overexpressing hESCs (“NPTX1over”) on day 7 following lentiviral transduction of NPTX1 in NPTX1over cells. Control cells were transduced with empty vector.

FIG. 8 is a bar graph showing the percentage of PAX6+ cells on day 7 of spontaneous differentiation following transduction of hESCs with NPTX1 (“NPTX1over”) or empty vector (“control”).

FIG. 9 shows a bar graph of the percentage of KDR+ mesoderm cells (left panel) and the fold change in mRNA expression of the markers brachyury (middle panel) and PAX6 (right panel) in control or NPTX1 overexpressing (“NPTX1 Over”) cells cultured in conditions for mesodermal cell differentiation at the indicated time points (“D”=day).

FIG. 10 shows line graphs of the percentage of CXCR4+c-Kit+endodermal cells (left panel), and the fold change in mRNA expression of SOX17 (middle panel) or PAX6 (right panel) in control or NPTX1 overexpressing (“NPTX1 Over”) cells cultured in conditions for endodermal cell differentiation, at the indicated time points, “D”=day.

FIG. 11 is a bar graph showing the fold change in gene expression of NANOG and PAX6 in spontaneously differentiating NPTX1 shRNA-transduced hESCs on day 7 following treatment with conditioned media (CM) from 293 kidney cells transduced with NPTX1 (“NPTX1 CM”) or with control vector (“control CM”).

FIG. 12 is a bar graph showing the percentage of PAX6+ cells in cultures of spontaneously differentiating NPTX1 shRNA-transduced hESCs on day 7 following treatment with conditioned media (CM) from 293 kidney cells transduced with NPTX1 (“NPTX1 CM”) or with control vector (“control CM”).

FIG. 13 depicts gene expression profiles of cells treated with NPTX1 shRNA or control, with genes grouped into categories (grouped by Gene Ontology, including: “Ribosome”, “Embryonic Appendage Morphogenesis”, “Chromatin Binding”, “Cell Junction Organization”, “DNA Recombination”, “Eye Development”, “Forebrain Development”, “Positive Regulation of Cell Activation”, “Cell Fate Commitment”, “Muscle Tissue Development”, “RNA Splicing”, “mRNA Processing”, “Regulation of Cell Migration”, “Regulation of Cell Development”, “Brain Development”, “Heart Development”, “CNS Development”, and “Protein Kinase Cascade”). The data are expressed as Z-scores, with bars extending from the center vertical line to the left indicating depletion of that category of genes, and bars extending to the right indicating enrichment. In each category, two bars are shown, the upper, black bar in each category corresponds to NPTX1 shRNA treated groups and the lower, white bar corresponds to controls.

FIG. 14 shows line graphs of mRNA expression (expressed as percent change from day 0) of pluripotency genes OCT-4, DNMT3B, E-Cadherin, and CD9 over time (days 1, 2, 3, 5, 7) during neural differentiation of control hESCs (indicated by circle symbols) and hESCs treated with NPTX1 shRNA (indicated by square symbols) to knock down NPTX1. Asterisks indicate statistical significant with p<0.05.

FIG. 15 shows line graphs of mRNA expression (expressed as percent change from day 0) of genes associated with neural development, including PAX6, DACH1, EMX2, and FABP7 over time (days 1, 2, 3, 5, 7) during neural differentiation of control hESCs (indicated by circle symbols) and hESCs treated with NPTX1 shRNA to knock down NPTX1 (indicated by square symbols). Asterisks indicate statistical significant with p<0.05.

FIG. 16 is a bar graph showing fold change in gene expression of NANOG, CRIPTO, SMAD2 and SMAD3 in hESCs transduced with NPTX1-specific shRNA2 (“NPTX1 shRNA”) or control (empty vector) transduced hESCs.

FIG. 17 is a line graph showing the fold change in CRIPTO gene expression in hESCs transduced with control (empty vector) or NPTX1 shRNA2 at the indicated time points (days, “D”).

FIG. 18 is a Western blot result in an NPTX1/CRIPTO coimmunoprecipitation experiment. The experimental conditions for each group are indicated for each of the indicated lanes (lane 1, culture media from control hESCs; lane 2, culture media from NPTX1 Overexpressing cells following differentiation toward neural cells (“NPTX1 Over Diff”); lane 3, culture media from control hESCs; lane 4, culture media from NPTX1 overexpressing hESCs; lane 5, cell membrane lysate from hESCs treated with control media (“Membrane Lysis Control Media”); lane 6, cell membrane lysate from hESCs treated with NPTX-His tag Media (“Membrane Lysis NPTX1-His tag Media”).

FIG. 19 is a bar graph showing the fold change relative to control in PAX6 gene expression in control (empty-vector transduced) hESCs, NPTX1 shRNA-transduced hESCs, and in NPTX1 shRNA-transduced hESCs treated with a CRIPTO blocking antibody (“Cripto Anti”).

FIG. 20 contains two bar graphs showing percent of hESCs expressing PAX6 (left graph) or SMAD1 (right graph) on day 7 of neural differentiation following differentiation in the standard differentiation protocol (“Noggin/SB431542”), or with the indicated modification to the standard protocol: Noggin substituted with an anti-CRIPTO blocking antibody (“Cripto-Ab/SB431542”), Noggin and SB431542 substituted with an anti-CRIPTO antibody (“Cripto-Ab”) or with BMP7 (“BMP7”) or with SB431542 alone (“SB431542”). In one group, NPTX1 overexpressing hESCs were cultured without addition of Noggin or SB431542 (“NPTX1 Over”).

FIG. 21 contains bar graphs showing relative endogenous and total mRNA expression levels of cMyc, Klf4, SOX2 and Oct4 in human fibroblasts (“hFibr”) and iPSCs (induced pluripotent stem cells) “iPSC1”), compared to hESCs.

FIGS. 22A and 22B are line graphs showing the fold change in mRNA expression levels of Nanog, NPTX1, and PAX6 on day 7 of neural differentiation in iPSCs and iPSCs treated with NPTX1 shRNA to knock down NPTX1 expression.

FIG. 23 is a bar graph showing the fold-change in mRNA expression levels of NPTX1, PAX6, and Nanog on day 7 in hESCs treated with recombinant C-reactive protein (“CRP”) or control (“vehicle”) and cultured in conditions for neural differentiation.

### DETAILED DESCRIPTION

### I. Overview

As discussed supra, there is a need for novel methods and compositions for deriving neural stem and progenitor cells (NSCs and NPCs) and/or other neural cells from stem cells. In particular, it is desirable to obtain neural cells from human stem cells, such as human embryonic stem cells (hESCs). In certain embodiments, the methods disclosed herein provide cell populations that are highly enriched for NSCs and/or NPCs, which can be used, e.g., for studying neural diseases.

In certain embodiments, the present disclosure provides methods for deriving neural cells, such as NSCs and NPCs, from stem cells using inhibitors of the Nodal/TGF beta signaling pathway. In a specific embodiment, the stem cells are ESCs, preferably hESCs. Inhibitors of the Nodal/TGF beta signaling pathway include for example and without limitation, NPTX1 protein and active fragments thereof, mutants and variants of NPTX1, and CRIPTO blocking antibodies. As an instance, and not by way of limitation, the Examples provided herein describe experiments where NPTX1 is determined to be necessary and sufficient to drive nervous system induction (e.g., differentiation of NSCs and NPCs) in hESCs.

Further, it is presently demonstrated herein that NPTX1 interacts with the TGF-beta coreceptor CRIPTO and regulates CRIPTO-mediated Nodal/TGF beta signaling. Thus, methods are provided for inhibiting the Nodal/TGF beta signaling pathway, which in turn leads to pluripotency exit (i.e., differentiation) of stem cells. In a specific embodiment, methods for deriving a neural stem cell from an ESC comprising administering NPTX1 to the ESC are provided.

In other embodiments, methods disclosed herein are useful for treating a condition associated with aberrant Nodal/TGF beta or BMP signaling. In certain embodiments, such methods comprise administering to a subject having such a condition an inhibitor of the Nodal/TGF beta signaling pathway or an inhibitor of the BMP signaling pathway (e.g., NPTX1). For example, methods disclosed herein may be useful for the treatment of cancer. NPTX1 expression has been reported in a variety of cancers. As TGF beta signaling, and in particular CRIPTO, is implicated in the progression of some tumors, e.g., breast and brain cancers, NPTX1, as demonstrated herein, is a protein that can be used to inhibit CRIPTO-dependent TGF beta signaling in the context of tumor progression or any other disease where TGF beta signaling has been implicated, such as but not limited to heart disease and Marfan syndrome.

In yet other embodiments, the present disclosure provides methods for maintaining stem cells, such as, e.g., ESCs, in an undifferentiated state by inhibiting NPTX1 expression. Without being bound by theory or mechanism, because NPTX1 is thought to work by inhibiting CRIPTO-dependent, TGF-beta/Nodal signaling, a pathway known to maintain pluripotency, inhibiting the expression of NPTX1 advantageously provides an efficient way to maintain stem cells in a pluripotent state by inhibiting spontaneous neural differentiation.

In yet other embodiments, NPTX1 can be used as a TGF beta inhibitor in pathologies where TGF beta signaling is known to be aberrant. NPTX1 is expressed in tissues other than in the nervous system, such as, e.g., in bone, and is also expressed in a variety of cancers including brain and breast, where aberrant TGF beta signaling has been implicated in cancer progression. Thus, regulating NPTX1 according to the methods described herein can slow down tumor progression.

In yet other embodiments, the present invention provides methods for increasing differentiation of stem or progenitor cells toward the mesodermal lineage, the method comprising administering to the stem or progenitor cell an inhibitor of the Nodal/TGF beta signaling pathway and/or an inhibitor of the BMP signaling pathway. The invention is based in part on the discovery that increased percentages of mesodermal cells can be obtained following differentiation of stem cell cultures in the presence of such inhibitors (e.g. a pentraxin protein, such as NPTX1 or CRP) compared to conventional methods of differentiation.

### II. Definitions

As used herein, the term “stem cell” refers to a cell that retains the ability to renew itself through mitotic cell division, and can differentiate into a diverse range of specialized cell types. The term “stem cell” includes by way of non-limiting examples, neural stem cells (NSCs), embryonic stem cells (ESCs), retinal pigment epithelial stem cells (RPESCs), induced pluripotent stem cells (iPSCs) and epiblast stem cells. As used herein, the term “embryonic stem cell (ESC)” refers to a stem cell derived from the inner cell mass of a blastocyst, an early-stage embryo. Human embryos reach the blastocyst stage 4-5 days post fertilization, at which time they consist of 50-150 cells. Human ESCs are characterized for example by high expression of OCT4, NANOG, SOX2, TRA-181, SSEA-4, and SSEA3, and low expression of markers of differentiated cells, such as but limited to Brachury, Sox17, Foxa2, Pax6, Otx2, and Sox1. Appropriate markers of ESCs of various species of origin or known, and can be readily determined by one of ordinary skill in the art.

The term “progenitor cell” as used herein refers to an undifferentiated cell that has the ability to differentiate into one or more different cell lineages, but is thought to have no or limited ability to self renew. Typically, a stem cell culture, such as, e.g., an hESC culture or NSC culture, will contain some progenitor cells in addition to stem cells. In some instances, progenitor cells derive from stem cells, during the process losing the ability to self-renew but maintaining the ability to differentiate into one or more different cell lineages. In other words, stem cells can give rise to progenitor cells.

As used herein, the terms “neural stem cell (NSC)” and “neural progenitor cell (NPC)” describe undifferentiated cells that can generate nervous system cells. NSCs have the ability to self renew, whereas NPCs are thought to have very limited or no ability to self renew. Markers of NSCs and NPCs include without limitation, combinations of one or more markers such as Nestin, Lex (CD-15), Musashi, Bmi-1, Sox1, Sox2, Hes1, Hes5, BLBP (brain lipid binding protein) and CD133, which markers are well known in the art. As used herein, “neural” means the nervous system and includes glial cells and neurons. The term “neural cell”, as used herein, includes NSCs, NPCs, neurons, glia, astrocytes, retinal neurons, photoreceptors, oligodendrocytes, olfactory cells, hair cells, supporting cells, and the like, and other cell types of the nervous system.

As used herein, the term “pentraxin polypeptide” includes full-length polypeptides and active fragments thereof, i.e., any portion of a pentraxin polypeptide (e.g., such as an NPTX1 polypeptide), which has an amino acid length that is shorter than the full-length pentraxin protein, which retains at least one biological activity of a pentraxin protein according to the present invention, in particular, the ability to induce neural differentiation of a stem cell and/or the ability to inhibit the Nodal/TGF-beta and/or BMP signaling pathway. Assays for determining whether an active fragment retains at least one biological activity of a pentraxin protein are described in more detail, infra.

As uses herein, the term “increasing or improving mesodermal differentiation” means increasing the percentage of mesodermal cells in a stem cell culture following differentiation under the appropriate conditions compared to a reference control group, such as, e.g., mesodermal cells cultured according to conventional methods (as described e.g., in Kennedy, et al. (2007) Blood 109:2679-2687). Preferably, a stem cell culture (e.g., ESC or iPSC culture) having increased and/or improved mesodermal differentiation as provided by the methods disclosed herein will typically consist of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% cells of the mesodermal lineage following culture in conditions for mesodermal differentiation in the presence of an inhibitor of the Nodal/TGFbeta and/or BMP signaling pathway (e.g., a pentraxin polypeptide, such as, but not limited to, NPTX1 or CRP).

The terms “neural induction” and “neural cell induction” are used interchangeably herein to mean the generation of a neural cell from another cell type, such as, e.g., from a stem cell.

As used herein, a “retinal pigment epithelial stem cell (RPESC)” is a stem cell that is activated from the adult human retinal pigment epithelium (RPE). RPESCs can be expanded many fold in vitro and produce a wide variety of progeny from diverse developmental lineages (including mesoderm and ectoderm). RPESCs are capable of producing retinal cells, and they also are capable of producing a much wider repertoire of progeny, including bone, muscle and adipocytes. These cells and how to identify and/or isolate them are described in detail in U.S. Patent Application Publication No. 2009/0274667 by Temple et al. Such cells can also be cultured according to the methods of the present invention.

As used herein, the terms “mutant” and “mutation” refer to any detectable change in genetic material (e.g., DNA) or any process, mechanism, or result of such a change. This includes gene mutations, in which the structure (e.g., DNA sequence) of a gene is altered, any gene or DNA arising from any mutation process, and any expression product (e.g., protein or enzyme) expressed by a modified gene or DNA sequence.

As used herein, the term “isolated” means that the referenced material is removed from the environment in which it is normally found. Thus, an isolated biological material can be free of cellular components, i.e., components of the cells in which the material is found or produced. Isolated nucleic acid molecules include, for example, a PCR product, an isolated mRNA, a cDNA, or a restriction fragment. Isolated nucleic acid molecules also include, for example, sequences inserted into plasmids, cosmids, artificial chromosomes, and the like. An isolated nucleic acid molecule is preferably excised from the genome in which it may be found, and more preferably is no longer joined to non-regulatory sequences, non-coding sequences, or to other genes located upstream or downstream of the nucleic acid molecule when found within the genome. An isolated protein can be associated with other proteins or nucleic acids, or both, with which it associates in the cell, or with cellular membranes if it is a membrane-associated protein. An isolated material may or may not be “purified,” as defined herein. The term “purified” as used herein refers to a material (e.g., a cell) that has been isolated under conditions that detectably reduce or eliminate the presence of other contaminating materials. Contaminants may or may not include native materials from which the purified material has been obtained. A purified material preferably contains less than about 90%, less than about 75%, less than about 50%, less than about 25%, less than about 10%, less than about 5%, or less than about 2% by weight of other components with which it was originally associated.

The terms “express” and “expression” mean allowing or causing the information in a gene or DNA sequence to become manifest, for example, producing an non-coding (untranslated) RNA or a protein by activating the cellular functions involved in transcription and translation of a corresponding gene or DNA sequence. A DNA sequence is expressed in or by a cell to form an “expression product” such as RNA or a protein. The expression product itself, e.g. the resulting RNA or protein, may also be said to be “expressed” by the cell. The term “expression system” means a host cell and compatible vector under suitable conditions, e.g. for the expression of a protein coded for by foreign DNA (the “expression construct”) carried by the vector and introduced to the host cell. By “expression construct” is meant a nucleic acid sequence comprising a target nucleic acid sequence or sequences whose expression is desired, operatively associated with expression control sequence elements which provide for the proper transcription and translation of the target nucleic acid sequence(s) within the chosen host cells. Such sequence elements may include a promoter and a polyadenylation signal. The “expression construct” may further comprise “vector sequences”. By “vector sequences” is meant any of several nucleic acid sequences established in the art which have utility in the recombinant DNA technologies of the invention to facilitate the cloning and propagation of the expression constructs including (but not limited to) plasmids, cosmids, phage vectors, viral vectors, and yeast artificial chromosomes. By “operatively associated with” is meant that a target nucleic acid sequence and one or more expression control sequences (e.g., promoters) are physically linked so as to permit expression of the polypeptide encoded by the target nucleic acid sequence within a host cell.

As used herein, the term “maintains the cell in an undifferentiated state” refers to preventing or minimizing the amount of cell differentiation, e.g., spontaneous differentiation in culture. In certain embodiments, such as e.g., when culturing stem or progenitor cells, the term also includes maintaining the ability of the cell to differentiate into one more different cell lineages. For example, a multipotent stem cell or progenitor cell that is maintained in an undifferentiated state will express markers associated with stem or progenitor cells, but will express no or low levels of markers associated with differentiating or differentiated cells, and will also maintain its ability to differentiate into one more different cell lineages, i.e., will remain multipotent. Thus, a unipotent keratinocyte progenitor cell that is maintained in the undifferentiated state, for example, will not differentiate into a keratinocyte (i.e., will remain a progenitor cell), but will maintain the ability to differentiate into a keratinocyte (e.g., under appropriate culture conditions that signal the cell to undergo such differentiation).

The term “subject,” “patient” or “individual” as used herein refers to an animal having an immune system, preferably a mammal (e.g., rodent, such as mouse). In particular, the term refers to humans. As used herein, the term “mammal” has its ordinary meaning, and specifically includes primates, and more specifically includes humans. Other mammals that may be treated for the presence of a tumor, or in which tumor cell growth may be inhibited, include, but are not limited to, canine, feline, rodent (racine, murine, lupine, etc.), equine, bovine, ovine, caprine, and porcine species.

“Treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving the disease, i.e., causing regression of the state, disorder or condition or at least one of its clinical or subclinical symptoms.

The term “pharmaceutically acceptable derivative” as used herein means any pharmaceutically acceptable salt, solvate or prodrug, e.g., ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5th Edition, Vol 1: Principles and Practice, which is incorporated herein by reference to the extent of teaching such derivatives. Preferred pharmaceutically acceptable derivatives are salts, solvates, esters, carbamates, and phosphate esters. Particularly preferred pharmaceutically acceptable derivatives are salts, solvates, and esters. Most preferred pharmaceutically acceptable derivatives are salts and esters.

As used herein the terms “therapeutically effective” and “effective amount,” used interchangeably, applied to a dose or amount refers to a quantity of a composition, compound or pharmaceutical formulation that is sufficient to result in a desired activity upon administration to an animal in need thereof. Within the context of the present invention, the term “therapeutically effective” refers to that quantity of a composition, compound or pharmaceutical formulation that is sufficient to reduce or eliminate at least one symptom of a disease or condition specified herein. When a combination of active ingredients is administered, the effective amount of the combination may or may not include amounts of each ingredient that would have been effective if administered individually. The dosage of the therapeutic formulation will vary, depending upon the nature of the disease or condition, the patient's medical history, the frequency of administration, the manner of administration, the clearance of the agent from the host, and the like. The initial dose may be larger, followed by smaller maintenance doses. The dose may be administered, e.g., weekly, biweekly, daily, semi-weekly, etc., to maintain an effective dosage level.

Therapeutically effective dosages can be determined stepwise by combinations of approaches such as (i) characterization of effective doses of the composition or compound in in vitro cell culture assays using tumor cell growth and/or survival as a readout followed by (ii) characterization in animal studies using tumor growth inhibition and/or animal survival as a readout, followed by (iii) characterization in human trials using enhanced tumor growth inhibition and/or enhanced cancer survival rates as a readout.

As used herein “combination therapy” or “adjunct therapy” means that a subject in need of treatment with a certain composition or drug is treated or given another composition or drug for the disease in conjunction with the first composition or drug. Combination therapy can be sequential therapy where the subject is treated first with one composition or drug and then the other, or alternatively, the two drugs can be given simultaneously. In either case, these drugs are said to be “coadministered.”

As used herein, the term “tumor” refers to a malignant tissue comprising transformed cells that grow uncontrollably, and that sometimes, though not necessarily always, is capable of metastasizing. As used herein, the term “tumor” encompasses cancer. The term “cancer” refers to all types of cancer, neoplasm or malignant tumors found in mammals, including without limitation leukemia, carcinomas and sarcomas. The terms “treating a tumor” and “inhibits/inhibiting tumor growth” are used interchangeably and refer to a decrease in the rate of tumor growth, and/or in the size of the tumor and/or in the rate of local or distant tumor metastasis in the presence of a composition of the invention, and/or any decrease in tumor survival, and can include treating cancer.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition. Cold Spring Harbor, N.Y.: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994. These techniques include site directed mutagenesis as described in Kunkel, Proc. Natl. Acad. Sci. USA 82: 488-492 (1985), U.S. Pat. No. 5,071,743, Fukuoka et al., Biochem. Biophys. Res. Commun. 263: 357-360 (1999); Kim and Maas, BioTech. 28: 196-198 (2000); Parikh and Guengerich, BioTech. 24: 4 28-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999), U.S. Pat. Nos. 5,789, 166 and 5,932,419, Hogrefe, Strategies 14. 3: 74-75 (2001), U.S. Pat. Nos. 5,702,931, 5,780,270, and 6,242,222, Angag and Schutz, Biotech. 30: 486-488 (2001), Wang and Wilkinson, Biotech. 29: 976-978 (2000), Kang et al., Biotech. 20: 44-46 (1996), Ogel and McPherson, Protein Engineer. 5: 467-468 (1992), Kirsch and Joly, Nuc. Acids. Res. 26: 1848-1850 (1998), Rhem and Hancock, J. Bacteriol. 178: 3346-3349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218. The skilled person will know and be able to use these and other techniques routine in the art to practice the present invention.

### III. Pentraxins

It is presently demonstrated that the secreted protein, NPTX1, is necessary and sufficient for neural induction. Further, it is demonstrated herein that CRP can induce hESCs cultured in neural differentiation conditions to upregulate NPTX1 and the neural differentiation marker PAX6. Thus, the present invention is based in part on the discovery that pentraxin polypeptides are capable of driving neural differentiation. The present invention encompasses all pentraxin polypeptides that have neural-generating ability. Preferred, but non-limiting examples of pentraxin polypeptides encompassed by the present invention include NPTX1, NPTX2, NPTXR, C-reactive protein (CRP), PTX3, SVEP and APCS. These proteins are secreted and form pentamers and decamers that bind to a wide variety of ligands such as bacteria, toxins, carbohydrates, and chromatin (see, Kirkpatrick et al. (2000) J Biol Chem 275:17786-17792). The ability of the pentraxin proteins to form big protein complexes that can bind chromatin make them highly intriguing in the context of early human neural development. The petraxin family of proteins is widely studied, and one of its members, CRP, has been used as a clinical diagnostic for decades for stress and inflammation.

The NPTX1 protein has been reported to be expressed in the adult brain, in particular the hippocampus and cerebral cortex. Studies have suggested that NPTX1 plays a role in regulating neuronal synaptic activity by binding to machinery involved in synaptic activity (Bjartmar et al. (2006) J Neurosci 26:6269-6281; Perin et al. (1996) Biochemistry 35:13808-13816). To date, however, it is not known what function NPTX1 may have, if any, in neural development. An NPTX1 knockout mouse was generated, and these mice appeared normal; however, it was suggested that NPTX2, a protein with greater than 90% homology to NPTX1 might compensate for the loss of NPTX1, thus masking the role of this protein at all stages (Kirkpatrick et al., supra).

Human NPTX1 has a preferred nucleic acid sequence with GenBank® Accession No. NM_{—}002522 (SEQ ID NO: 1) and a preferred amino acid sequence with GenBank® Accession No. NP_{—}002513 (SEQ ID NO: 2). Murine Nptx1 has a preferred nucleic acid sequence with GenBank® Accession No. NM_{—}008730 (SEQ ID NO: 3) and a preferred amino acid sequence with GenBank® Accession No. NP_{—}032756 (SEQ ID NO: 4). Isolated or recombinant full length NPTX1 polypeptide can be administered to a cell according to the present methods in order, e.g., to induce neural differentiation of stem cells such as, e.g., ESCs. NPTX1 obtainable from any suitable species, preferably a mammalian species (e.g., human, murine, primate, etc.), is contemplated for use according to the present methods and compositions.

Human CRP has a preferred nucleic acid sequence with GenBank® Accession No. NM_{—}000567.2 (SEQ ID NO: 5) and a preferred amino acid sequence with GenBank® Accession No. CAA39671.1 (SEQ ID NO: 6). NPTX2 has a preferred nucleic acid sequence with GenBank® Accession No. NM_{—}002523.2 (SEQ ID NO: 7) and a preferred amino acid sequence with GenBank® Accession No. NP_{—}002514.1 (SEQ ID NO: 8). NPTXR has a preferred nucleic acid sequence with GenBank® Accession No. NM_{—}014293.3 (SEQ ID NO: 9) and a preferred amino acid sequence with GenBank® Accession No. NP_{—}055108.2 (SEQ ID NO: 10). PTX3 has a preferred nucleic acid sequence with GenBank® Accession No. NM_{—}002852.3 (SEQ ID NO: 11) and a preferred amino acid sequence with GenBank® Accession No. NP_{—}002843.2 (SEQ ID NO: 12). APCS has a preferred nucleic acid sequence with GenBank® Accession No. NM_{—}001639.3 (SEQ ID NO: 13) and a preferred amino acid sequence with GenBank® Accession No. NP_{—}001630.1 (SEQ ID NO: 14).

Also contemplated for use in the present methods are active polypeptide fragments of the pentraxin proteins disclosed, above (i.e., any portion of a pentraxin protein (e.g., such as NPTX1 protein)), which has an amino acid length that is shorter than the full-length pentraxin protein, which retains at least one biological activity of a pentraxin protein according to the present invention, in particular, the ability to induce neural differentiation of a stem cell and/or the ability to inhibit the Nodal/TGFbeta and/or BMP signaling pathway.

Thus, in a preferred embodiment, for example, an active polypeptide fragment of NPTX1 or other pentraxin polypeptide will retain the ability to induce neural differentiation of a stem cell. The skilled artisan will be able to determine which fragments of NPTX1 or other pentraxin polypeptide retain the desired activity, e.g., by determining whether the aforesaid fragment retains the ability to bind to CRIPTO and/or whether the fragment inhibits the Nodal/TGF beta and/or BMP signaling pathway. As shown in the present Examples, NPTX1 co-immunoprecipitates with CRIPTO, and, while not intending to be limited herein by one particular theory or mechanism, is thought to inhibit the CRIPTO-dependent Nodal/TGF beta signaling pathway, as well as the BMP signaling pathway, thereby inducing pluripotency exit and differentiation of stem cells. Therefore, as an example, an NPTX1 fragment that retains the ability to bind to CRIPTO is an “active fragment” of NPTX1, as used herein. The skilled artisan will understand that other methods for determining whether a fragment of NPTX1 or other pentraxin protein (e.g., CRP, NPTX2, NPTXR, etc.) is an active fragment, e.g., by testing the fragment in another biological assay designed to analyze a desired activity (e.g., ability to induce neural differentiation of a stem cell (e.g., as measured by PAX6 upregulation on day 7 of culture in conditions for neural differentiation), may also be used.

Also contemplated for use herein are mutants or variants of pentraxin polypeptides, e.g., NPTX1 and CRP that retain at least one biological activity of NPTX1, in particular, the ability to induce differentiation of stem cells. As described above for active NPTX1 fragments, the ordinary skilled artisan will be able to determine whether a mutant or variant of NPTX1 retains the desired biological activity using a relevant biological assay, e.g., to determine whether the mutant or variant protein retains the ability to bind to CRIPTO and/or to inhibit the Nodal/TGF beta signaling pathway. Also contemplated herein are mutants or variants of other pentraxin proteins, as described above, that retain at least one function of the wild-type pentraxin protein, preferably the ability to drive neural differentiation of stem cells and/or the ability to induce NPTX1 upregulation and/or the ability to induce PAX6 upregulation in stem cells by day 7 of culture in conditions for neural differentiation. In certain embodiments, NPTX1 and other pentraxin proteins encompassed by the present invention can be administered to a cell or subject as a protein. In other embodiments, NPTX1 can be administered as an expression construct encoding an NPTX1 polypeptide, wherein a cell administered the construct expresses the NPTX1 polypeptide encoded by the construct. Expression constructs of the present invention may comprise vector sequences that facilitate the cloning and propagation of the expression constructs. A large number of vectors, including plasmid and fungal vectors, have been described for replication and/or expression in a variety of eukaryotic and prokaryotic host cells. Standard vectors useful in the current invention are well known in the art and include (but are not limited to) plasmids, cosmids, phage vectors, viral vectors, and yeast artificial chromosomes. The vector sequences may contain a replication origin for propagation in E. coli; the SV40 origin of replication; an ampicillin, neomycin, or puromycin resistance gene for selection in host cells; and/or genes (e.g., dihydrofolate reductase gene) that amplify the dominant selectable marker plus the gene of interest. For example, NPTX1, an active fragment thereof, or a mutant or variant of NPTX1 can be expressed using E. coli bacteria, and does not need to be modified post-translationally to be active. Any proteins encompassed by the present invention can be expressed as recombinant protein, or isolated from a naturally occurring source, or purchased commercially, when available.

### IV. Regulation of Nodal/TGF Beta and BMP Signaling

In certain embodiments, methods for inhibiting the Nodal/TGF beta signaling pathway and/or the BMP signaling pathway and/or for inducing stem cell differentiation, e.g., into neural cells, are provided. In a preferred embodiment, the method comprises inhibiting CRIPTO expression and/or function.

Human CRIPTO, also known as teratocarcinoma-derived growth factor 1 (TDGF1), has a preferred nucleic acid sequence with GenBank® Accession No. NM_{—}003212 (SEQ ID NO: 15) and a preferred amino acid sequence with GenBank® Accession No. NP_{—}003203 (SEQ ID NO: 16). Human CRIPTO has another preferred nucleic acid sequence with GenBank® Accession No. NM_{—}001174136 (SEQ ID NO: 17) and another preferred amino acid sequence with GenBank® Accession No. NP_{—}001167607 (SEQ ID NO: 18). Murine Cripto, also known as teratocarcinoma-derived growth factor 1 (Tdgf1) has a preferred nucleic acid sequence with GenBank® Accession No. NM_{—}011562 (SEQ ID NO: 19) and a preferred amino acid sequence with GenBank® Accession No. NP_{—}035692 (SEQ ID NO: 20).

CRIPTO is a developmental oncoprotein and a member of the epidermal growth factor-CRIPTO, FRL-1, Cryptic family of extracellular signaling molecules. In addition to having essential functions during embryogenesis, CRIPTO is highly expressed in tumors and promotes tumorigenesis. During development, CRIPTO acts as an obligate coreceptor for transforming growth factor beta (TGF-beta) ligands, including nodals, growth and differentiation factor 1 (GDF1), and GDF3. CRIPTO is essential for Nodal/TGF beta signaling. CRIPTO/Nodal signaling is transmitted via the transcription factors SMAD2 and SMAD3, and this pathway is thought to be critical for maintenance of ESCs in an undifferentiated state and ESC pluripotency. Further, blockade of CRIPTO-mediated Nodal signaling results in neural differentiation of hESCs by loss of SMAD expression and in turn lower NANOG expression (Lonardo et al., (2010) Stem cells (Dayton, Ohio) 28, 1326-1337.

The first step in neural development involves the initial specification of a neuronal fate from undifferentiated ectoderm. Neural fate acquisition in vertebrate development is thought to occur through inhibition of the TGF-beta and BMP signaling pathways. See, Hemmati-Brivanlou, A. and Melton, D. A. (1994). Cell 77, 273-281. Thus, the BMP signaling pathway is also a target for driving neural differentiation according to the methods of the present invention.

### V. Stem and Progenitor Cells

In certain embodiments, the present methods are useful for obtaining a neural cell from a stem cell, or for maintaining a stem cell in an undifferentiated state. Non-limiting examples of stem cells and related progenitor cells that may be cultured according to the methods of the invention include, e.g., skin stem cells, spermatagonial stem cells, hair follicle stem cells, cancer stem cells, bone marrow stem cells, gut stem cells, hematopoietic stem cells, adipose stem cells, mouse embryonic stem cells, human embryonic stem cells, retinal pigment epithelial stem cells, mesenchymal stem cells, epiblast stem cells, renal stem cells, amniotic stem cells, umbilical blood stem cells, endothelial stem cells, neural crest stem cells, and induced pluripotent stem cells (iPSCs).

In a preferred embodiment, the stem cells are embryonic stem cells (ESC), and even more preferably, human embryonic stem cells (hESC). Stem and progenitor cells may be derived by the skilled artisan, and such methods are known in the art. Many stem cells are also commercially available or available from cell banks, such as, e.g., the WiCell Reseach Institute, National Stem Cell Bank, Madison, Wis.

Progenitor cells related to the above stem cells (i.e., derived from such stem cells) may also be cultured according to the present invention. Any stem or progenitor stem cells now known or to be discovered may also be cultured according to the methods of the present invention.

The present methods may be used for culturing any mammalian stem and/or progenitor cell, such as, but not limited to, human, rat, pig, sheep, mouse, or non-human primate stem and/or progenitor cells. Stem cells can be derived according to any suitable method known in the art [see, e.g., Thomson et al. (1998) Science 28:1145-47; Amit et al. (2000) Dev Biol. 2:271-78; and Cowan et al. (2004) N Engl J Med 350:1353-1356; see also, U.S. Pat. Nos. 5,843,780; 6,200,806; and 7,029,913 (all to Thomson)].

In one aspect of the invention, the present methods are also useful for deriving NSCs and/or NPCs from ESCs. NSCs and NPCs have the potential to differentiate into neural cells, such as, e.g., neurons, glia, astrocytes, retinal neurons, photoreceptors, oligodendrocytes, olfactory cells, hair cells, supporting cells, and the like. NSCs and NPCs which may be cultured according to the methods described herein can be identified by the expression of certain markers, such as one or more of Nestin, Lex (CD-15), Musashi, Bmi-1, Sox1, Hes1, Hes5, BLBP, and CD133. NPCs can also express high levels of helix-loop-helix transcription factors NeuroD, Atoh1, and neurogenin1 and neurogenin2 NSC cultures typically contain a mixture of NSCs and NPCs, and both may be cultured according to the methods of the present invention.

### VI. Cell Culture Methods

For culturing stem and/or progenitor cells, appropriate culture medium and culture methods are known and described in the art. For example, cells can be cultured in serum free DMEM/high-glucose supplemented with N2 and B27 solutions and growth factors. Typically cells are incubated at 37° C., and 5% CO₂ in tissue culture treated wells. Optionally, cells can be cultured in the presence of feeder cells, such as mouse embryonic fibroblasts. See, e.g., Amit et al., supra; Fasano et al. (2010) Cell Stem Cell 6:336-47; Ludwig et al. (2006) Nat Methods 8:637-46; Bendall et al. (2007) Nature 448:1015-21; Qian et al. (1997) Neuron 1:81-83; Fasano et al. (2007) Cell Stem Cell 1:87-99; and Shen et al. (2004)Science 304:1338-40. Specific culture conditions are readily determined and adjusted by the ordinarily skilled artisan.

Typically, ESCs are grown until density is deemed suitable for the appropriate experiments being carried out by the investigator (typically one week). At this point, ESCs are passaged either as single cells or cell aggregates onto MEF feeders, or tissue culture treated plastic dishes coated with an extracellular matrix (Fasano et al., 2010, supra). Typically, NSCs and/or NPCs are grown until density is deemed suitable for the appropriate experiments being carried out by the investigator (typically one week). At this point, NSCs and/or NPCs are passaged as either single, dissociated cells or cell aggregates onto tissue culture treated plastic dishes coated with an extracellular matrix or non-tissue culture treated plates with no extracellular matrix when floating NSC (neurosphere) cultures are needed (Fasano et al., 2007, supra).

Neural cell induction can be carried out, e.g., as described in Chambers et al. (2009) Nat Biotechnol 27:275-280. Briefly, stem cells, such as WA-09 cells or any other suitable source of stem cells, are disaggregated, washed and pre-plated on gelatin in the presence of ROCK inhibitor. Nonadherent cells are removed and replated on suitable matrix coated dishes in mouse embryonic fibroblast (MEF) conditioned stem cell differentiation media (CM) containing knock out serum replacement (KSR) media with 10 nM TGF-β inhibitor (SB431542) and 500 ng/mL of Noggin and spiked with 10 ng/mL of FGF-2 and ROCK-inhibitor. ROCK inhibitor is then withdrawn, and the stem cells are allowed to expand in CM for 3 days or until they are nearly confluent. Upon day 5 of differentiation, increasing amounts of N2 media (see, Fasano et al., 2010, supra) (25%, 50%, 75%) is added to the KSR media every two days while maintaining 500 ng/mL of Noggin and TGF-β inhibitor. Any suitable method for neural induction is contemplated by the present disclosure.

In a specific embodiment, the method for inducing neural cells includes addition of NPTX1 to the stem cell culture with or without other active agents, such as, e.g., those described above (e.g., TGF-β inhibitor and/or Noggin and/or FGF-2). In a preferred embodiment, NPTX1 alone (i.e., without other active agents) is administered to a cell.

### VII. Assaying Cell Differentiation

Generally, it is possible to determine if a stem or progenitor cell is “maintained” as a stem or progenitor cell (i.e., maintained in an undifferentiated state) by determining whether it continues to express one or more markers associated with such cells. For example, markers of human embryonic stem cells, include, but are not limited to the markers OCT4, NANOG, TRA-181, SOX2, SSEA-4, and/or SSEA3. Markers of NSCs and NPCs include, without limitation, Nestin, Lex (CD-15), Musashi, BMI-1, SOX1, HES1, HES5, BLBP, and CD133. In addition, an ESC that is maintained in an undifferentiated state generally will not express, or will express relatively low levels of markers indicative of differentiation such as Brachyury, SOX17, FOXA2, PAX6, OTX2, and SOX1. An NSC that is maintained in an undifferentiated state will not express, or will express relatively low levels (compared to a differentiated cell) of markers including, without limitation, Tuj 1, S100β, Galactocerebroside and/or MBP (myelin basic protein). For instance, as shown in the present Examples, hESCs treated with NPTX1 downregulate expression of NANOG and upregulate expression of SOX1 and PAX6. The above examples are not intended to be limiting and these and other markers indicative of differentiated and undifferentiated states are known and routinely used in the art for characterizing stem cells and differentiated cells.

In other embodiments, stem cells may be differentiated toward endodermal lineages. Sox17 is an example of an endodermal marker. Other, non-limiting examples of endodermal markers include, e.g., goosecoid, AFP1, and FOXA2.

The present invention advantageously provides a method for improving differentiation of stem or progenitor cells toward mesodermal linages. Cells of the mesodermal lineage are, for example, c-Kit+CXCR4+cells that express KDR (also known as VEGFR2). Other, non-limiting examples of markers expressed on cells of the mesodermal lineage include, e.g., CD31, EOMES, HAND1, and HAND2.

The above-described and other markers are well known in the art and contemplated for use herein. Moreover, non-limiting examples of tissues formed by cells of the mesodermal lineage include, e.g., blood, heart, skeletal muscle, smooth muscle, liver, pancreas, gut, glands, pancreas, and respiratory tract. The present invention thus provides methods for improving mesodermal differentiation, i.e., increasing the percentage of mesodermal cells in a stem cell culture following differentiation under the appropriate conditions).

Non-limiting, exemplary markers for identification of stem cells, NSCs/NPCs and differentiated cells (e.g., neural cells or cells of the endodermal or mesodermal lineages), as described above, are listed in the table, below, along with their GenBank Accession Numbers (for the human sequences).

| | | GENBANK | SEQ |
|---|---|---|---|
| MARKER | CELL TYPE | ACCESSION NO. | ID NO |
| PAX6 | NEURAL | NM_000280 | 21 |
| SOX1 | NPC/NSC | NM_005986.2 | 22 |
| NANOG | NEURAL | NM_024865.2 | 23 |
| KLF4 | STEM CELL | NM_004235.4 | 24 |
| KDR | MESODERMAL | NM_002253 | 25 |
| C-KIT | MESODERMAL | NM_000222.2 | 26 |
| SOX17 | MESODERMAL | NM_022454.3 | 27 |
| SMAD1 | ESC | NM_005900.2 | 28 |
| SMAD2 | STEM CELL | NM_005901.4 | 29 |
| SMAD3 | STEM CELL | NM_005902.3 | 30 |
| GBX2 | NEUROECTODERM | NM_001485.2 | 31 |
| SOX2 | NEUROECTODERM | NM_003106.2 | 32 |
| CXCR4 | ENDODERM | NM_001008540.1 | 33 |
| DNMT3B | STEM CELL | NM_006892.3 | 34 |
| E-CADHERIN | STEM CELL | NM_004360.3 | 35 |
| CD9 | STEM CELL | NM_001769.3 | 36 |
| DACH1 | NEURAL | NM_080759.4 | 37 |
| EMX2 | NEURAL | NM_004098.3 | 38 |
| FABP7 | NEURAL | NM_001446.3 | 39 |
| cMYC | STEM CELL | NM_002467.4 | 40 |
| BRACHYURY | MESODERMAL | NM_003181 | 41 |

Other undifferentiated and differentiated cells encompassed by the present invention will also express similar and/or different markers characteristic of the undifferentiated state and/or of a differentiated state of the cell. Such markers are known or may be readily determined by the skilled artisan, and the expression of such markers may be analyzed to determine whether the cell is maintained in an undifferentiated state or is differentiated into, e.g., a neural cell or a cell of the mesodermal or endodermal lineage.

A variety of methods can be utilized to determine whether, for example, neural differentiation of a stem cell or differentiation into another cell type or lineage has been induced and/or whether a stem cell has been maintained in an undifferentiated state. Such methods are well known in the art, and include, for example and without limitation, RT-PCR and/or Northern blot for analysis of gene expression, and Western blot, ELISA, immunohistochemistry, and/or fluorescence activated cells sorting (FACS) for analysis of protein expression of any of the above described markers. Such analyses are also useful for characterizing differentiating/differentiated and undifferentiated cells of non-human species of origin, and appropriate markers for such species, which may be the same, some of the same, or different than those described above for human cells, are known and readily determined by the ordinary skilled artisan.

It is to be understood that the methods of the present invention can be used for culturing many types of undifferentiated cells, such as but not limited to stem cells, progenitor cells, neural cells such as immature neural cells, etc., from any species of origin. A person of skill in the art can readily determine which markers are appropriate for characterizing the specific cell being cultured.

For stem cells, specifically, assessment of stem cell differentiation (or maintenance of stem cells in an undifferentiated state) also can be determined by analysis of morphological features of the stem cell culture. hESCs exhibit high nucleus to cytoplasm ratio with prominent nucleoli, and are rounded and typically grow in colonies that lie tightly packed together. The borders of these colonies are very tight, rigid and well-defined. NSCs exhibit a flat, in some cases pavemented morphology. NSCs tend to grow as clones, or in groups held very closely together, where they might take on a square or roughly triangular appearance (Temple, 1989; Thomson et al., supra). Additionally, NSCs can be put in culture as floating aggregates known as neurospheres. After one week, these neurospheres are dissociated into single cells and replated in the same conditions. A properly maintained NSC line will continue to generate new spheres at the same rate or higher than the previous passage. A deficit in NSC maintenance would result in reduced neurosphere formation after passage (Fasano et al, 2007, supra).

### VIII. Compositions and Pharmaceutical Formulations

The compositions described herein can be administered to a cell, such as but not limited to a stem cell, e.g., an ESC, to induce neural differentiation of the ESC. Thus, in certain embodiments, a composition comprising an inhibitor of the Nodal/TGF beta signaling pathway is provided. In a specific embodiment, a composition comprises an isolated NPTX1 polypeptide. In another embodiment, a composition comprises an expression construct encoding NPTX1 polypeptide. In still another embodiment, a composition comprises a CRIPTO blocking antibody. In one embodiment, a composition comprises a small molecule inhibitor of the Nodal/TGF beta signaling pathway.

Small molecules are typically organic, non-peptide molecules, having a molecular weight less than 10,000 Da, preferably less than 5,000 Da, more preferably less than 1,000 Da, and most preferably less than 500 Da. This class of modulators includes chemically synthesized molecules, for instance, compounds from combinatorial chemical libraries. Synthetic compounds may be rationally designed or identified utilizing screening methods. Alternative appropriate modulators of this class are natural products, particularly secondary metabolites from organisms such as plants or fungi, which can also be identified by screening compound libraries for tumor-killing activity. Methods for generating and obtaining small molecules are well known in the art (Schreiber, Science 2000; 151:1964-1969; Radmann et al., Science 2000; 151:1947-1948).

By way of example, and without limitation, SB43152 is a broad TGF beta antagonist that can be used to inhibit the Nodal/TGF beta signaling pathway according to the methods described herein.

Certain compositions described herein can be administered to a cell, such as, but not limited to, a stem cell, e.g., an ESC, to maintain the cell in an undifferentiated state. Thus, in certain embodiments, a composition of the invention comprises an inhibitor of NPTX1. Non-limiting examples of NPTX1 inhibitors include blocking antibodies and small molecules.

Compositions can be formulated for administration in any convenient way for use in human or veterinary medicine. The active agents described herein, e.g., NPTX1 or NPTX1 inhibitor, can be incorporated into liposomes, microemulsions, micelles, unilamellar or multilamellar vesicles, erythrocyte ghosts or spheroblasts. In one embodiment, the active agent can be delivered in one or more vesicles, including as a liposome (see Langer, Science, 1990; 249:1527-1533; Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.).

While not intending to be bound by theory, in certain embodiments, the use of NPTX1 for inducing neural differentiation of stem cells is advantageous in that only a single, transient treatment with NPTX1 is required for neural induction. However, in certain embodiments, it may be desirable to deliver NPTX1 or another Nodal/TGF beta inhibitor over a prolonged period of time. Thus, in some embodiments, a composition can be a sustained release composition. A “sustained release composition” can include any suitable vehicle that releases one or more factors (e.g., NPTX1) over a period of time. As used herein, sustained release compositions are suitable for culturing with undifferentiated cells, such as but not limited to stem and/or progenitor cells. Non-limiting examples of sustained release compositions of the invention include microspheres (e.g., poly(DL-lactide-co-glycolide) (PLGA) microspheres), anhydrous poly-vinyl alcohol (PVA), millicylinders, alginate gels, biodegradable hydrogels, complexing agents and nanoparticles. [See, e.g., Ashton, et al. (2007) Biomaterials, 28, 36, 5518; Drury, J. L. et al. (2003) Biomaterials; 24:4337-4351; U.S. Pat. No. 7,226,617 to Ding et al.; Simmons, C. A. et al. (2004) Bone; 35:562-569; Zhu, G. et al. (2000) Nat Biotech; 18:52-57; Derwent et al.(2008) Trans Am Ophthalmol Soc 106:206-13.] Mechanical methods for time-release are also included, for example a mechanical device can be used to provide a continuous, or near continuous, sustained supply of growth factor to a cell culture over time and thereby maintain the stem or progenitor cells at a stable level of differentiation. Sustained release compositions suitable for administration to a cell or subject are described in detail in U.S. Patent Application Publication No. 2010/0021422 and in U.S. Patent Application No. 61/360,741 (both by Temple et al.).

The subject invention also concerns the use of NPTX1, other inhibitors of the Nodal/TGF beta signaling pathway, and NPTX1 inhibitors in the preparation of pharmaceutical formulations. While it is possible to use a composition for therapy as is, it may be preferable to administer compositions as pharmaceutical formulations, e.g., in admixture with a suitable pharmaceutical excipient, diluent, or carrier selected with regard to the intended route of administration and standard pharmaceutical practice. Pharmaceutical formulations comprise at least one active compound, or a pharmaceutically acceptable derivative thereof, in association with a pharmaceutically acceptable excipient, diluent, and/or carrier. The excipient, diluent and/or carrier must be “acceptable” in the sense of being compatible with the other ingredients of the formulation and not deleterious to the recipient thereof.

As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

Pharmaceutical formulations may comprise, for example, and without limitation, an inhibitor of the Nodal/TGF beta signaling pathway and a pharmaceutical carrier. Pharmaceutical formulations may also comprise, e.g., NPTX1 or an active fragment, mutant or variant thereof, or an inhibitor of NPTX1, and a pharmaceutical carrier.

### IX. Kits

The compositions described herein can be provided in a kit. The kit can include one or more compositions of the invention (e.g., NPTX1 or active fragment thereof) suitable for inducing neural differentiation of stem cells; and, optionally, (b) informational material. In one embodiment, the kit provides an NPTX1 inhibitor suitable for maintaining stem cells in an undifferentiated state, and, optionally informational material.

The informational material can be descriptive, instructional, marketing or other material that relates to the methods described herein and/or to the use of the sustained release composition for the methods described herein. The kits can also include paraphernalia for administering one or more compounds to a cell (e.g., pipette, dropper, etc.).

The informational material of the kits is not limited in its form. In many cases, the informational material (e.g., instructions) is provided in printed matter, such as in a printed text, drawing, and/or photograph, such as a label or printed sheet. However, the informational material can also be provided in other formats, such as Braille, computer readable material, video recording, or audio recording. Of course, the informational material can also be provided in any combination of formats.

The kit can include one or more containers for the composition(s). In some embodiments, the kit contains separate containers, dividers or compartments for the composition and informational material. For example, the composition can be contained in a bottle, tube or vial, and the informational material can be contained in a plastic sleeve or packet. In other embodiments, the separate elements of the kit are contained within a single, undivided container. For example, the sustained release composition is contained in a bottle, tube or vial that has attached thereto the informational material in the form of a label.

### X. Methods of Treatment

In certain embodiments, methods for inducing neural differentiation in vivo are provided. For example, compositions can be administered to a site of neural cell damage, e.g., spinal cord injury, to induce neural differentiation from stem or progenitor cells. In certain embodiments, a composition, e.g., an NPTX1-containing composition, is administered with a stem cell directly to a nervous system site to promote regeneration of nervous system cells at a site of nervous system injury. In a specific embodiment, a composition comprising NPTX1 and a stem cell is administered to a site of spinal cord injury In other embodiments, a composition comprising NPTX1 is administered to a site of neural cell damage or nervous system injury and/or systemically, and another composition comprising a stem cell is administered to the same site or to another site, or systemically (i.e., the NPTX1 and stem cell are administered separately to the same or different sites, by the same or different routes of administration).

In certain embodiments, methods for treating a condition associated with aberrant Nodal/TGF beta signaling are provided. In one embodiment, the condition is a tumor. In another embodiment, the condition is heart disease.

Tumors include without limitation leukemias, lymphomas, myelomas, plasmacytomas, and the like; and solid tumors. Examples of solid tumors that can be treated according to the invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilns' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Further, as discussed above, the term tumor encompasses cancer. Exemplary cancers include without limitation cancer of the breast, brain, cervix, colon, head & neck, liver, kidney, lung, non-small cell lung, melanoma, mesothelioma, ovary, sarcoma, stomach, uterus and Medulloblastoma. Additional examples include, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

In other embodiments, a composition of the invention (e.g., comprising a therapeutically effective amount of an inhibitor of the Nodal/TGFbeta and/or BMP signaling pathway) may be used to treat heart disease, or diseases of other mesodermal lineages, such as blood, heart, skeletal muscle, smooth muscle, etc. For example, the compositions of the present invention may be used to treat muscle atrophy diseases such as muscular dystrophy, stroke, blood and vessel aneurisms.

In yet other embodiments, a composition of the invention (e.g., comprising an inhibitor of the Nodal/TGF-beta and/or BMP signaling pathway) may be used to treat congenital disease and/or prevent miscarriage. In a specific embodiment, an antagonist of CRP may be administered to a pregnant patient to prevent miscarriage. Such treatment might be appropriate for a patient suffering, e.g., from repeated miscarriages.

In certain embodiments, compositions may be administered with another agent or drug. For example, a composition for inhibiting Nodal/TGF beta signaling in a tumor can be coadministered with a chemotherapeutic agent. As used herein, the terms “chemotherapeutic agent” and “chemotherapeutic drug” are used interchangeably and refer to a compound that is capable of inhibiting, disrupting, preventing or interfering with cell growth and/or proliferation. Chemotherapeutic agents include, for example and without limitation, taxanes such as taxol, taxotere or their analogues; alkylating agents such as cyclophosphamide, isosfamide, melphalan, hexamethylmelamine, thiotepa or dacarbazine; antimetabolites such as pyrimidine analogues, for instance 5-fluorouracil, cytarabine, capecitabine, and gemcitabine or its analogues such as 2-fluorodeoxycytidine; folic acid analogues such as methotrexate, idatrexate or trimetrexate; spindle poisons including vinca alkaloids such as vinblastine, vincristine, vinorelbine and vindesine, or their synthetic analogues such as navelbine, or estramustine and a taxoid; platinum compounds such as cisplatin; epipodophyllotoxins such as etoposide or teniposide; antibiotics such as daunorubicin, doxorubicin, bleomycin or mitomycin, enzymes such as L-asparaginase, topoisomerase inhibitors such as topotecan or pyridobenzoindole derivatives; and various agents such as procarbazine, mitoxantrone, and biological response modifiers or growth factor inhibitors such as interferons or interleukins. Other chemotherapeutic agents include, though are not limited to, a p38/JAK kinase inhibitor, e.g., SB203580; a phospatidyl inositol-3 kinase (P13K) inhibitor, e.g., LY294002; a MAPK inhibitor, e.g. PD98059; a JAK inhibitor, e.g., AG490; preferred chemotherapeutics such as UCN-01, NCS, mitomycin C (MMC), NCS, and anisomycin; taxoids in addition to those describe above (e.g., as disclosed in U.S. Pat. Nos. 4,857,653; 4,814,470; 4,924,011, 5,290,957; 5,292,921; 5,438,072; 5,587,493; European Patent No. 0 253 738; and PCT Publication Nos. WO 91/17976, WO 93/00928, WO 93/00929, and WO 96/01815.

### XI. Administration

Compositions and formulations can be administered topically, parenterally, orally, by inhalation, as a suppository, or by other methods known in the art. The term “parenteral” includes injection (for example, intravenous, intraperitoneal, epidural, intrathecal, intramuscular, intraluminal, intratracheal or subcutaneous).

Compositions may be administered once a day, twice a day, or more often. Frequency may be decreased during a treatment maintenance phase of the disease or disorder, e.g., once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art. More generally, dose and frequency will depend in part on recession of pathological signs and clinical and subclinical symptoms of a disease condition or disorder contemplated for treatment with the present compounds.

It will be appreciated that the amount of active agent (e.g., NPTX1 or NPTX1 inhibitor) required for use in treatment will vary with the route of administration, the nature of the condition for which treatment is required, and the age, body weight and condition of the patient, and will be ultimately at the discretion of the attendant physician or veterinarian. Compositions will typically contain an effective amount of the active agent(s), alone or in combination. Preliminary doses can be determined according to animal tests, and the scaling of dosages for human administration can be performed according to art-accepted practices.

Exemplary dosages of NPTX1 for administration to humans range from about 0.0001 mg/kg to about 10 mg/kg of body weight, although lower or higher concentrations are possible.

Length of treatment, i.e., number of days, will be readily determined by a physician treating the patient; however, the number of days of treatment may range from 1 day to about 20 days, or longer.

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### EXAMPLES

### Materials and Methods

The following materials and methods were employed in the Examples described herein.

Cells and Culture Conditions

Human embryonic stem cells (hESCs) (WA-09; passages 35-45, (Global Stem)) were cultured on mouse embryonic fibroblasts (MEFs) plated at 12-15,000 cells/cm². A medium of DMEM/F12, 20% knockout serum replacement (GIBCO), 0.1 mM β-mercaptoethanol, 6 ng/mL FGF-2 (R&D Systems, Minneapolis, Minn.) was changed daily. Cells were passaged using 6 U/mL of dispase (Worthington Biochemical Corp., Lakewood, N.J.) in hESCs media, washed and re-plated at dilutions of 1:5 to 1:10. 293 kidney cells were obtained from Invitrogen (Carlsbad, Calif.).

Neural Induction (“Dual SMAD Inhibition Protocol”)

Feeder free neural induction was carried out as previously described (Chambers et al., supra). Briefly, hESC cultures (WA-09 cells) were disaggregated using Accutase (Innovative Cell Technologies, Inc., San Diego, Calif.) for 20 minutes, washed using hESC media and pre-plated on gelatin for 1 hour at 37° C. in the presence of ROCK inhibitor (Sigma, Carlsbad, Calif.) to remove MEFs. The nonadherent hESCs were washed and plated at a density of 20,000 cells/cm² on BD Matrigel™ matrix (BD Biosciences, San Diego, Calif.) coated dishes in MEF conditioned hESCs media (CM) spiked with 10 ng/mL of FGF-2 and ROCK-inhibitor (Sigma). The ROCK inhibitor was withdrawn, and hESCs were allowed to expand in CM for 3 days or until they were nearly confluent. The initial differentiation media conditions included knock out serum replacement (KSR) media with 10 nM TGF-β inhibitor (SB431542, Tocris) and 500 ng/mL of Noggin (R&D Systems). Upon day 5 of differentiation, increasing amounts of N2 media (Fasano et al., 2010, supra) (25%, 50%, 75%) was added to the KSR media every two days while maintaining 500 ng/mL of Noggin and TGF-β inhibitor.

Spontaneous Differentiation

The hESCs cultures were disaggregated using Accutase for 20 minutes, washed using hESC media and pre-plated on gelatin for 1 hour at 37° C. in the presence of ROCK inhibitor to remove MEFs. The nonadherent hESCs were washed and plated at a density of 17,500 cells/cm² on matrigel-coated dishes in MEF conditioned hESCs media (CM) spiked with 10 ng/mL of FGF-2 and ROCK-inhibitor. The next day the media was changed to KSR medium with no growth factor. Media was changed every other day for 7-10 days.

Quantitative Real-Time PCR

Total RNA was extracted using an RNeasy kit (Qiagen). For each sample, 1 μg of total RNA was treated for DNA contamination and reverse transcribed using Quantitect first strand kit (Qiagen). Amplified material was detected using Taqman probes and PCR mix (ABI) on a Mastercycler RealPlex2 (Eppendorf). All results were normalized to a HPRT control and were from 3 technical replicates of 3 independent biological samples at each data point.

Microscopy

Cells were fixed using 4% paraformaldehyde for 15 minutes, washed with PBS, permeabilized using 0.3% Triton X in PBS, and blocked using 10% normal goat serum. Primary antibody used for microscopy was PAX6 (DSHB).

Western Blots and Co-Immunoprecipitation (IP)

For WB, cells were lysed and ran on SDS page gels (Invitrogen) and blotted with the following antibodies: anti-SMAD3 and anti-Phospho-SMAD3 (Cell Signaling). Blots were developed by using the appropriate horseradish peroxidase conjugated secondary antibody. For the co-IPs, the media was collected and performed using a Mu1tiMACS kit from Miltenyi Biotec Inc. The media was incubated with immobilized anti-CRIPTO antibody or rabbit polyclonal IgG isotype control antibody, and the bound material was then eluted and run on a gel for Western blot. To determine whether NPTX1 was bound to CRIPTO, the blots were stained with anti-NPTX1 antibody. Antibodies used were the following: anti-NPTX1 (gift from Dr. Paul Worley), anti-NPTX1 (Abcam) and anti-CRIPTO (Abcam). Blocking anti-CRIPTO antibody was obtained from Abcam.

Vector Design and Lentiviral Production

NPTX1 open reading frame (ORF) was generated by PCR. This fragment was cloned into the BamH1 and NotI sites of the lentiviral vector, pCDH-EF1-MCS-IRES GFP(SBI Biosciences). A third generation lentiviral vector (Lois et al. (2002) Science 295:868-872) was modified to express an NPTX1 shRNA from the H1 promoter as described (see, Fasano et al., 2007, supra; Ivanova et al. (2006) Nature 442:533-538). The shRNA sequences in the vectors had the following sequences: shRNA1: AGT AAA GAG TGG AGA AAG A (SEQ ID NO: 42); shRNA2: ACG TGT AGG TTG AGA ACA A (SEQ ID NO: 43); and shRNA3: GAG AAA GGT CAG AAA GAC A (SEQ ID NO: 44). The shRNA-expressing lentiviral plasmid was co-transfected with plasmids pVSV-G and pCMVd8.9 into 293FT cells (Invitrogen). Virus-containing media were collected, filtered, and concentrated by ultracentrifugation. Viral titers were measured by serial dilution on NIH 3T3 cells (ATCC Deposit No. CRL-1658) followed by flow cytometric analysis after 72 hours.

Generation of BF1 shRNA and Over-Expressing Human ES Lines

hESCs (WA-09; passages 35) were dissociated and plated on Matrigel with the ROCK inhibitor as single cells. 24 hrs post plating the ESCs were transduced with either control (empty vector), NPTX1 shRNA, or NPTX10RF containing vectors. One (1) week later, GFP-expressing colonies were manually picked and plated on MEFs. Cells were then expanded, tested for mycoplasma.

Conditioned Media and ELISA

293FTs were transduced with NPTX1 and control vector. 48 hours post transduction, media was collected every day for 5 days. This media was used for differentiation of hESCs. To assay levels of NPTX1 during neural induction, hESCs were differentiated to NSCs and NPCs, and PAX6+NSCs and NPC and media was harvested at various time points. Using a human Netrin-1 ELISA kit (Abgent) according to the manufacturer's protocol, NPTX1 protein levels were detected.

iPSC Generation and Characterization.

Adult human fibroblasts from an apparently healthy 21-year old female were purchased from Coriell (#AG09309). iPSC were derived by transduction of the human fibroblasts with four retroviral supernatants containing OCT4, SOX2, KLF4 and c-MYC, according to (Carvajal-Vergara et al. (2010) Nature 465:808-812). Expression of pluripotency markers and viral integration were verified according to standard verification procedures (see, Maherali N, Hochedlinger K. Guidelines and techniques for the generation of induced pluripotent stem cells. Cell Stem Cell 2008; 3:595-605). Briefly, the resulting iPSC lines were analyzed by immunostaining and FACS analysis for the expression of pluripotency markers, including Oct4 (Cell Signaling C30A3), Sox2 (SCBT sc-17319), KLF4 (SCBT sc-20691), c-myc (SCBT sc-764), Tra-1-60 (BD 560173), Nanog (R&D AF1997), SSEA4 (BD 560308) and SSEA1 (BD 560127). qPCR analysis confirmed that these iPSC expressed a similar expression pattern of endogenous pluripotency genes compared to hESCs, the gold standard of pluripotency. Primers used were as described in Takahashi et al. (2009) PloS one 4:e8067. The iPSC clones were able to give rise to teratoma in vivo.

Microarray Analysis

Normalization and model-based expression measurements were performed with GC-RMA. GC-RMA, as well as analytical and annotation packages, are available as part of the open-source Bioconductor project (www.bioconductor.org) within the statistical programming language R (http://cran.r-project.org/)(Team, R.D.C. (2011). R: A Language and Environment for Statistical Computing). Time of Maximum (TOM) values were calculated in R as previously reported (Venezia et al. (2004) PLoS Biol 2:e301.). Briefly, expression profiles for each gene were analyzed by regressing the normalized expression values using polynomial least squares regression. ANOVA was performed on the coefficients of regression to identify genes with significant time patterns (p<0.05). The smooth curve fitting assumed the expression trajectory for each gene followed a continuous time pattern. The class of fourth-degree polynomials was chosen for the fits, because it was the highest degree polynomial which did not interpolate the time point means. Enrichment of a Gene Ontology category (as described at http://www.geneontology.org/) was calculated utilizing the genes uniquely changing in either H1 Control or NPTX1 shRNA cells over the time course. A Z-test was used to calculate a p-value determining the probability that the association between the genes in either group and the Gene Ontology category was explained by chance alone. A two sample Z-test was used to calculate a p-value determining the probability that the association between the genes in either group was enriched compared to the other group.

Endoderm and Mesoderm Differentiation and FACS

Prior to differentiation, iPSCs were feeder-depleted by passaging onto matrigel. To generate EBs, iPSC were dissociated into small clusters of 10-20 cells, then plated in low attachment plates (Costar, Corning) with the appropriate differentiation media (see, Nostro, et al. (2011) Development 138:861-871). For endoderm differentiation: serum-free media supplemented with 100 ng/ml activin A for 5 days (Id.). For mesoderm differentiation: StemPro34 (Invitrogen) media supplemented with 20 ng/ml BMP4 and 10 ng/ml bFGF (D1-D3), then 10 ng/ml VEGF and 10 ng/ml bFGF (D4-D6) (Kennedy et al., 2007, supra). EBs were collected at different time points and dissociated in 0.25% Trypsin-EDTA, then stained with the appropriate antibodies. Stained cells were analyzed on a FACS Aria 2 Cell Sorter (BD Biosciences). Data were analyzed using FlowJo software.

Western Blot, Co-Immunoprecipitation, and ELISA

ELISA for NPTX1 was performed using a kit from USCN Life Sciences (Wuhan, China) according to the manufacturer's protocol. For CRIPTO co-immunoprecipitation, media from either H1 control cells or NPTX1 over-expressing cells was isolated during the neural induction protocol. CRIPTO antibody (Abcam, ab19917 (Boston, Mass.)) in conjunction with μMACS Protein G Microbeads (Miltenyi Biotec (Auburn, Calif.), 130-071-101) and MACS Separation Columns (Miltenyi Biotec, 130-042-701) was used to immunoprecipitate CRIPTO. All experiments were performed according to manufacturer's protocol and Rabbit polyclonal IgG was used as an isotype control. Samples were run in each well of a 4-12% Bis-Tris mini-gel (Invitrogen, NP0335Box) with MES buffer (Bio-Rad, #101-0789). Samples were transferred to a PVDF membrane (Invitrogen,(Carlsbad, Calif.) LC2005) in Transfer buffer (Invitrogen, NP0006-1). Membranes were placed on a rocking shaker in blocking buffer (TBS, 0.1% Tween, 5% BSA) for 1 hour at room temperature. Membranes were then placed on a rocking shaker overnight at 4° C. in staining buffer (TBS, 0.1% Tween, 3% BSA) and stained with NPTX1 (1 μg/mL, kindly provided by Paul Worley, Johns Hopkins) Membranes were washed 5× with TBST and placed in blocking buffer for 1 hour at room temperature. Membranes were then stained with anti-rabbit IgG-HRP (Abcam, ab6721, 1:3000) in staining buffer for 1 hour at room temperature. Finally, membranes were washed 5× with TBST, then stained with WB luminol reagent (SCBT, sc-2048) for one minute, and imaged on a ChemiDoc XRS (Bio-Rad, #170-8070).

Immunocytochemistry Antibodies

Antibodies used for performing immunocytochemistry, methods for which are described in Fasano et al., 2007, and 2010, supra, were obtained commercially as follows: PAX6 (Developmental Studies Hybridoma Bank (Iowa City, Iowa)), SOX2 (Santa Cruz Biotechnology (SCBT) (Santa Cruz, Calif.)), Nestin (Chemicon (Temecula, Calif.)), Nanog (SCBT), OCT-4 (SCBT), SMAD3 and SMAD1 (Cell Signaling Technology, Inc., Danvers, Mass.), FOXG1 (NeuraCell, Rensselaer, N.Y.), SSEA-4, SSEA-3, SSEA-1 (BD Pharmingen (San Diego, Calif.)). Alexa secondary antibodies were purchased from Invitrogen.

Statistical Analysis

Results shown are mean+SEM. Asterisks and pound signs identify experimental groups that were significantly different from control groups by a t-test, one way ANOVA, or two way ANOVA with a Bonferroni correction for multiple comparisons (p-value, 0.05), where applicable. All experiments were performed with a minimum of n=3 except for iPSC NPTX1 knockdown, which was n=2.

### Example 1

### NPTX1 Expression in Differentiating Neural Cells

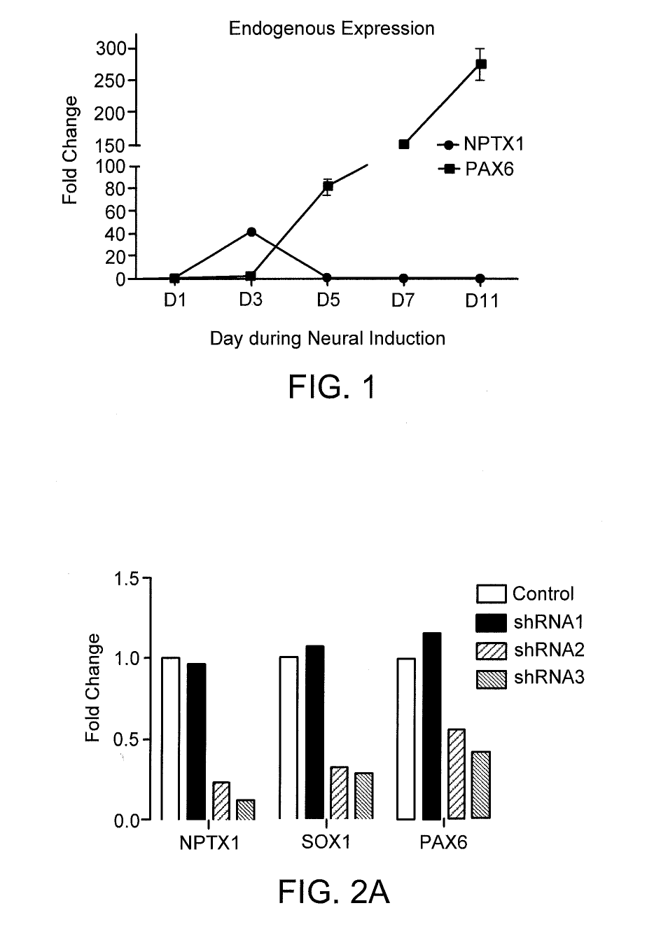

Neural cells were derived from WA-09 hESCs and RNA was isolated along an 11-day time course. NPTX1 Gene expression was determined using RT-PCR. NPTX1 gene expression peaked transiently around day 3 then rapidly returned to baseline expression level (FIG. 1). This transient peak in expression occurred before the upregulation of the neural marker PAX6 (FIG. 1).

### Example 2

### Effect of Knock Down of NPTX1 on Neural Differentiation

Lentiviral shRNAs specific for knock down of NPTX1 were made and hESC were transduced. It was verified that 2 out of 3 shRNAs (shRNA2 and shRNA3) successfully knocked down NPTX1 at the message level at day 3 of neural differentiation (a time point when NPTX1 expression is highest). shRNA1 did not knock down NPTX1 as was thus used as a negative control in certain subsequent experiments.

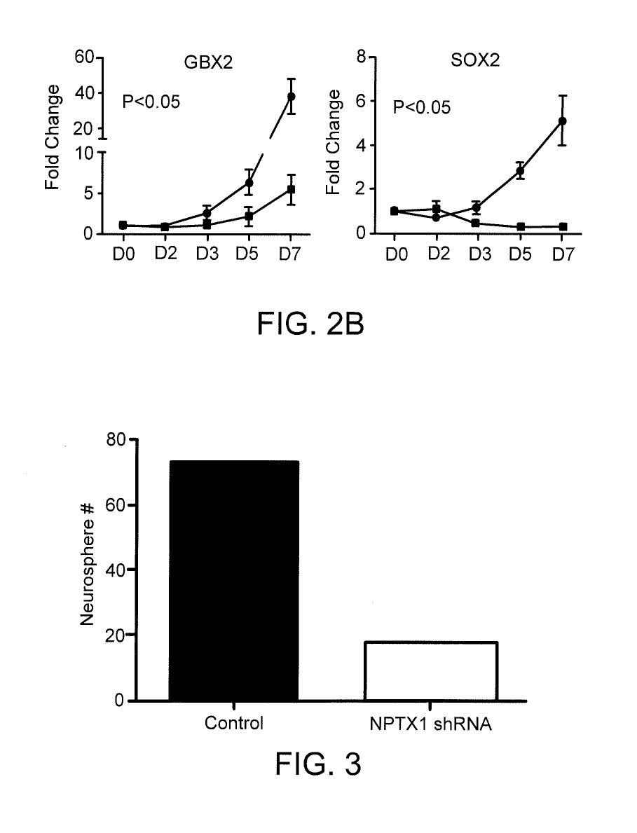

To assess NPTX1 function, WA-09 hESCs transduced with NPTX1 shRNA or control vector were differentiated towards the neural lineage (i.e., into NSCs and NPCs) and expression of NPTX1 and the neural genes SOX1 and PAX6 was assessed at day 7 (FIG. 2A). Further, expression levels of the neuroectodermal markers GBX2 and SOX2 were assessed on days 0, 2, 3, 6 and 7 (FIG. 2B).

Cells treated with shRNA2 or with shRNA3 had significantly reduced levels of expression of the neural genes SOX1 and PAX6 compared to control (empty vector transduced cells), as determined by the level of mRNA expression (FIG. 2A). This effect was verified at the protein level using an anti-PAX6 antibody. As another control, the shRNA that did not successfully knock down NPTX1 (shRNA1) was included in this experiment and, as expected, had no effect on the levels of SOX1 and PAX6 genes (FIG. 2A).

Along the neural differentiation time course (days 0-7), the cells were monitored for expression of the early neuroectodermal markers GBX2 and SOX2 by qRT-PCR. In the shRNA treated cells (treated with shRNA3), there was a drastic reduction in expression of these markers (with days 5 and 7 showing statistical significance), suggesting that NPTX1 regulates the early stages of neural commitment from hESCs.

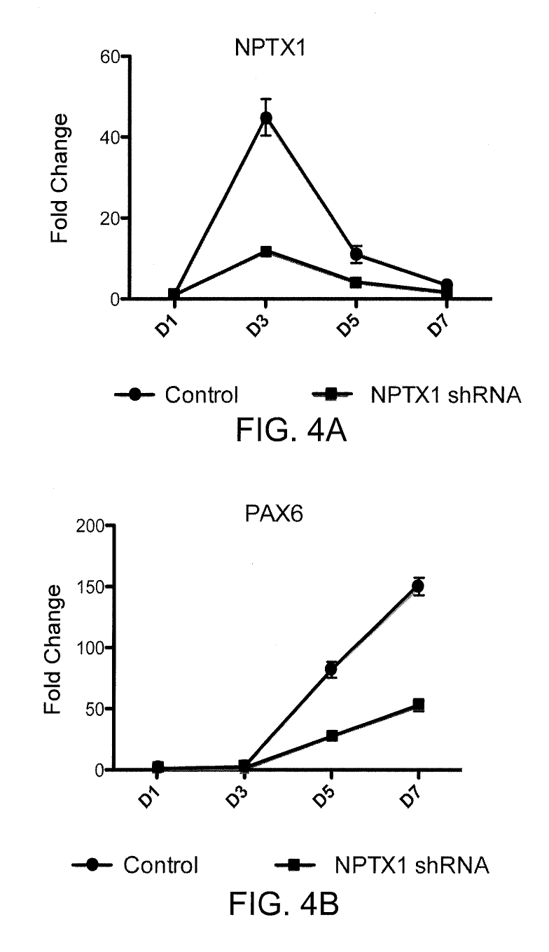

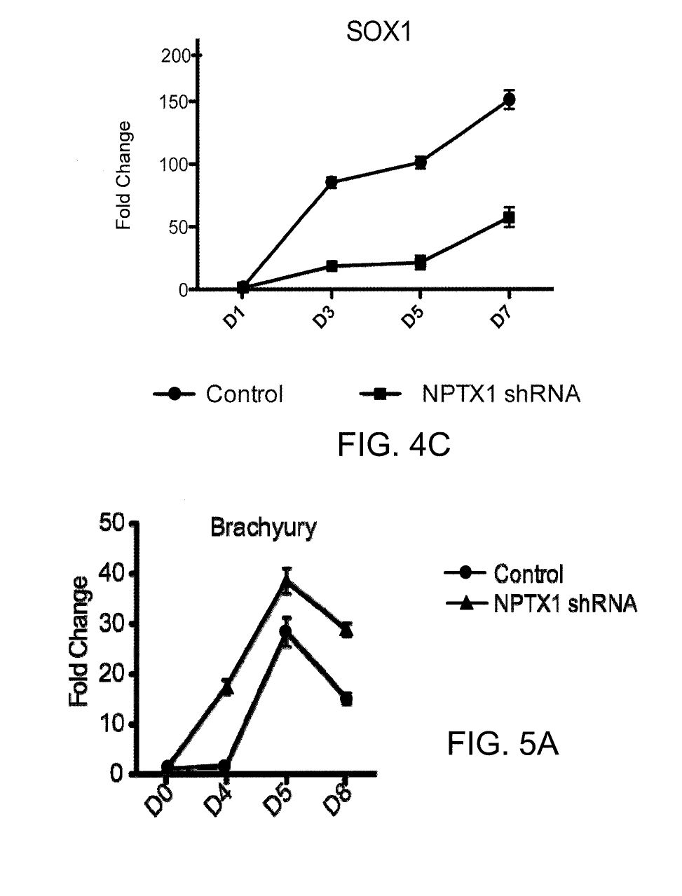

To assess the neural character of the cells generated, the resulting cells were isolated at day 7 of differentiation and subjected to a neurosphere assay. As compared to the control, cells generated with reduced levels of NPTX1 (using shRNA2) resulted in a significant reduction in the number of neurospheres (FIG. 3). To expand on these results, RNA was then isolated along the time course of neural differentiation to characterize expression trends of the neural genes PAX6 and SOX1 in the knockdowns. The fold change in expression of NPTX1 in control cells and in cells treated with NPTX1 shRNA2 is shown in FIG. 4A. In control conditions, the neural genes PAX6 and SOX1 started to appear around day 4 (FIGS. 4B and 4C, respectively). When NPTX1 was reduced using shRNA2 (FIG. 4A), however, gene expression of PAX6 and SOX1 was much delayed and at much lower levels compared to control (FIGS. 4B and 4C).

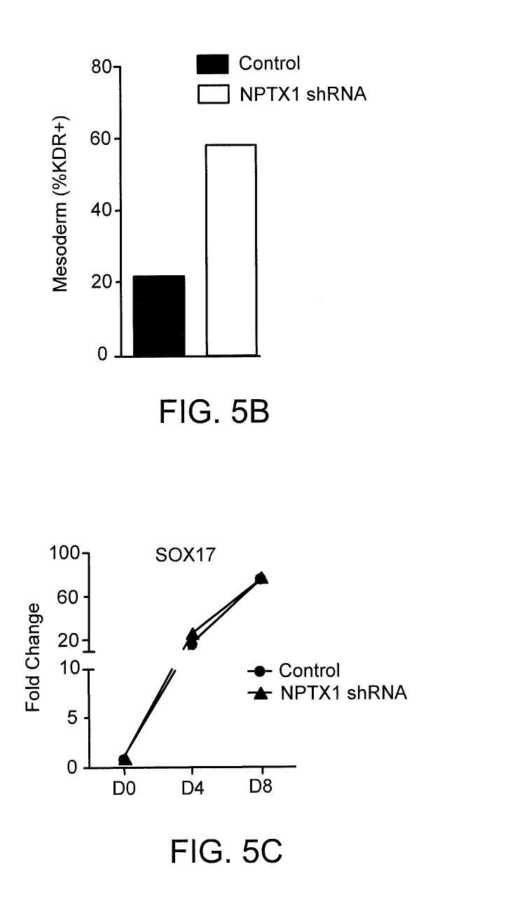

It was also possible that NPTX1 regulated the general differentiation potential of the hESCs, and knocking it down would inhibit its ability to differentiate into any lineage, not just neural. To test this possibility, hESCS treated with NPTX1 shRNA, as above, were differentiated towards endodermal or mesodermal fates as previously described (Fasano et al., 2010, supra). It was found that cells treated with NPTX1 shRNA were able to give rise to brachyury-expressing cells (FIG. 5A), as determined by qRT-PCR, as well as KDR+ (FIG. 5B), as determined by flow analysis, indicating that the cells differentiated toward the mesodermal fate, despite knock down of NPTX1. Interestingly, the NPTX1 shRNA treated cells had higher percentages of mesodermal populations (KDR+, brachyury-expressing cells) compared to the control. At no time point measured for either mesodermal or endodermal differentiation was expression of PAX6 found, indicating that no neural differentiation was occurring in the absence of NTPX1. Additionally, the cells expressed the endodermal marker SOX17, indicating that knock down of NPTX1 using shRNA allowed the cells to give rise to endoderm equally as well as controls (FIG. 5C). Thus, these experiments demonstrated the important role of NPTX1 in neural differentiation, as well as identified a novel method for increasing production of mesodermal and endodermal cells from ESCs in vitro by inhibiting NPTX1 during differentiation.

### Example 3

### Effect of Overexpression of NPTX1 on Neural Differentiation

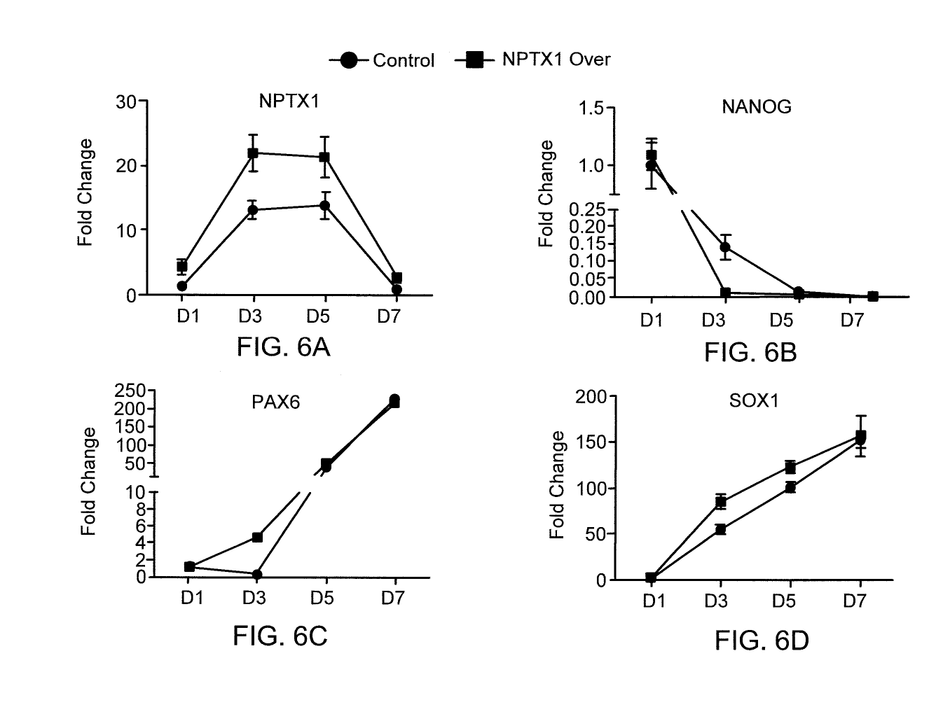

To determine if NPTX1 would enhance neural differentiation, a lentiviral vector was used to force expression of NPTX1 in hESCs (“NPTX1over”). Following lentiviral transduction of the hESCs with NPTX1, the NPTX1over cells, along with an empty vector-transduced line (control), were differentiated to the neural lineage and RNA was collected along the time course. As seen in the control, NPTX1 expression peaked early and rapidly dropped off as expression of the neural markers PAX6 and SOX1 increased (FIGS. 6A, 6C and 6D, respectively). In the NPTX1over cells, NPTX1 levels were elevated from day 1, and PAX6 and SOX1 levels were increased earlier, as determined by qRT-PCR (FIGS. 6A, 6C and 6D, respectively). Further, overexpression of NPTX1 caused hESCs to exit pluripotency faster than control cells, as indicated by decreased levels of NANOG expression in NPTX1over cells (FIG. 6B). This effect was confirmed by immunostaining, which demonstrated that 27% of the NPTX1over cells were PAX6+cells by day 3, whereas only 5.1% of the control cells were PAX6+. On day 5, 59% of the NPTX1over cells were PAX6+, whereas only 24% of the control cells were Pax6+. These data demonstrated that NPTX1 enhances the rate of hESC differentiation into neural cells under directed differentiation conditions.

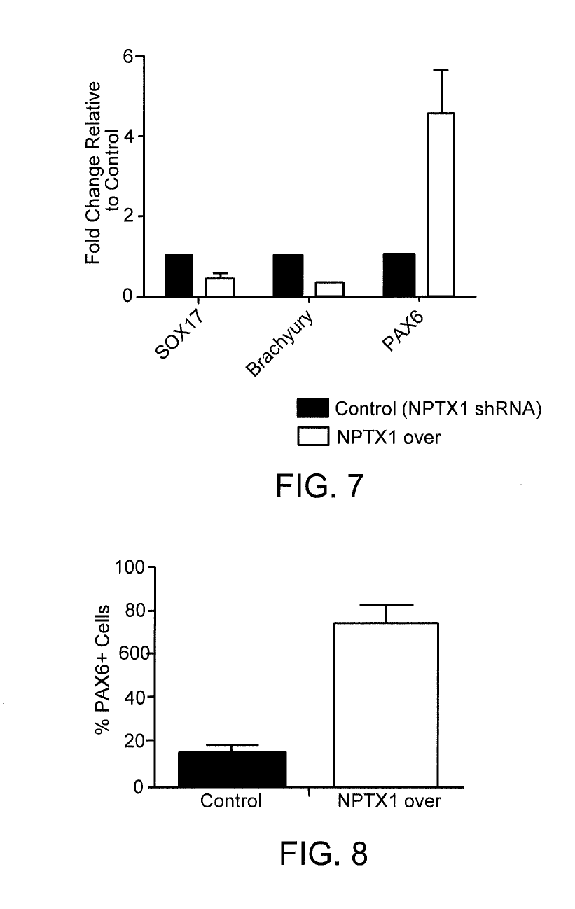

To test whether NPTX1 was sufficient to drive neural differentiation, NPTX1 over cells were allowed to spontaneously differentiate in a KSR-based medium with no exogenous growth factors. After one (1) week in culture, NPTXover cells exhibited robust PAX6 expression with a decrease in Brachyury and SOX17 expression (markers of mesoderm and endoderm respectively) (FIG. 7). hESCs transduced with empty vector (“control”) demonstrated the opposite result; specifically, a decrease in PAX6 expression and an increase in brachyury expression (FIG. 7). The increased neural differentiation in NPTX1 over cells was confirmed at the protein level by immunostaining of PAX6. The percentage of PAX6+cells was significantly higher in NPTX1 over cells compared to the control cells (FIG. 8).

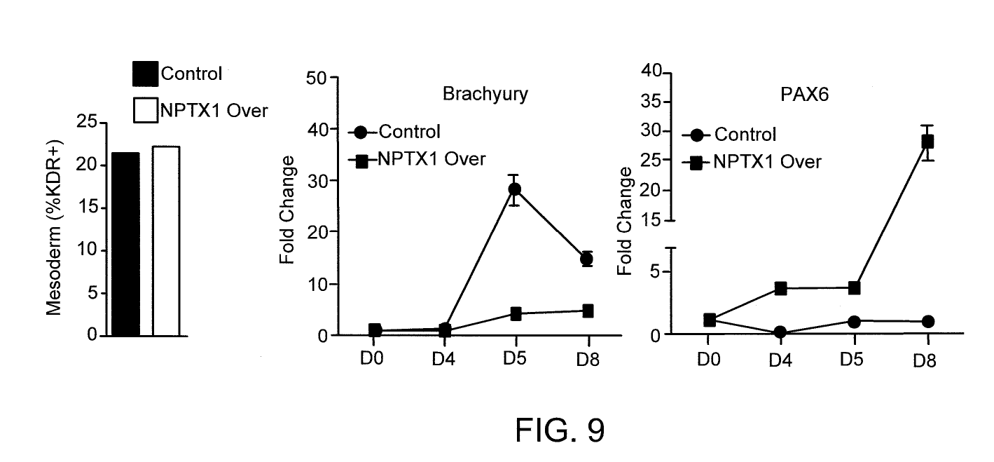

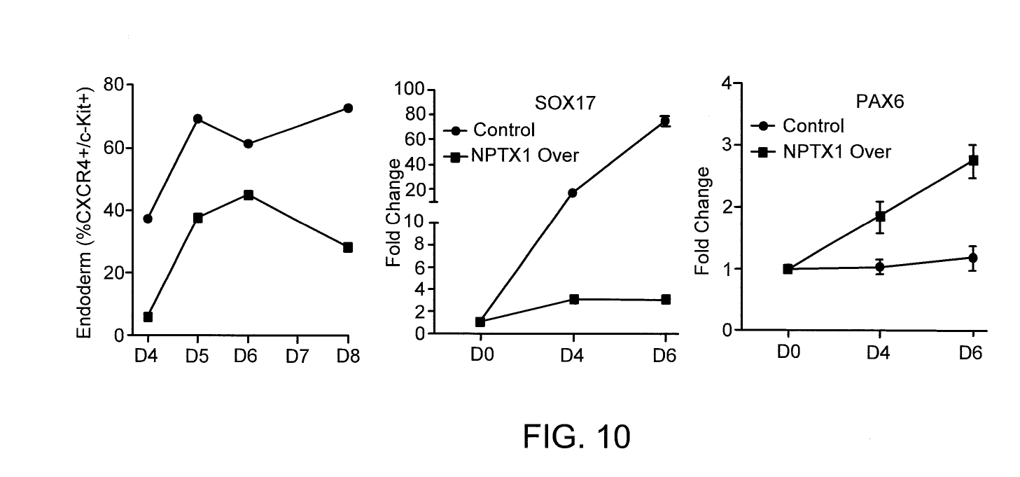

Next, the ability of NPTX1 to drive differentiation of NPTX1 over cells toward neural lineages in culture conditions designed to drive differentiation into other lineages (e.g., mesoderm and endoderm) was tested, in order to determine the relative “strength” of NPTX1 for driving neural differentiation. Using flow cytometry and qRT-PCR, it was determined that NPTX1 overexpressing cells were able to be driven to differentiate into both mesoderm and endoderm; however, in both differentiation conditions, the NPTX1 overexpressing cells gave rise to more neural cells, compared to control, as measured by PAX6 expression. In particular, NPTX1over cells cultured in conditions specific for mesodermal differentiation expressed lower levels of mesodermal marker brachyury at days 5 and 8, and expressed higher levels of neural marker PAX6 (FIG. 9). Similarly, NPTX1 over cells cultured in conditions specific for endodermal differentiation, differentiated into a cell population having a greater percentage of CXCR4+/cKit+ cells (endodermal cells), as determined by flow cytometry, and expressed lower levels of SOX17 mRNA, and higher levels of neural marker PAX6 mRNA on days 4 and 6 (FIG. 10). These results indicated that NPTX1 strongly compels neural induction in hESCs.

### Example 4

### Conditioned Medium Containing NPTX1 Drives Neural Differentiation

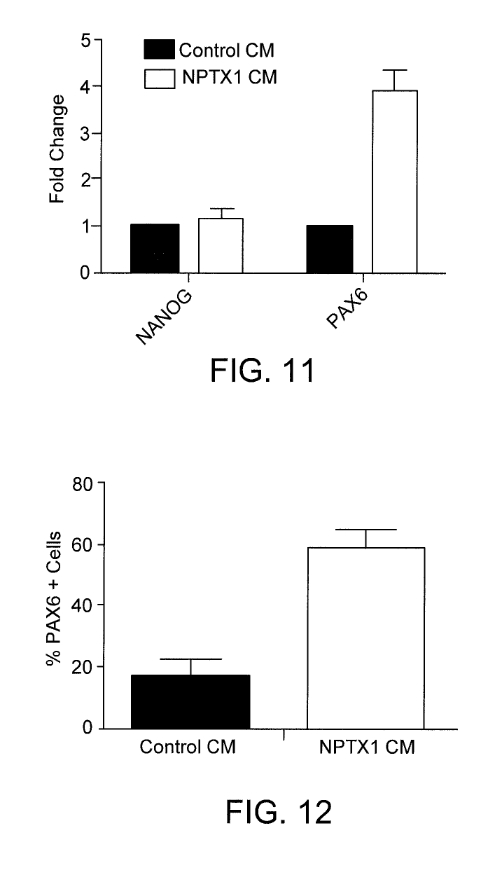

In order to test whether the observed effect of NPTX1 on neural differentiation could be mimicked without genetic manipulation, 293 kidney cells were transduced with NPTX1 and a control vector. The conditioned media (CM) was isolated and the CM from NPTX1-transduced 293 kidney cells contained NPTX1 (which is a secreted protein). The CM was then added to hESCs in culture and the cells were then left to differentiate spontaneously for 7 days. Cells that were treated with the NPTX1 CM had higher PAX6 mRNA message levels at day 7 compared to cells treated with the control CM (FIG. 11). By day 3 the cells treated with NPTX1 CM also appeared more columnar than the cells treated with control CM and at both days 5 and 7, the cultures treated with NPTX1 CM had significantly more PAX6+cells compared to control treated cells (2.2% vs. 22.3% at day 5 and 19.5% vs. 73.2% at day 7) (FIG. 12, showing day 7 results).

### Example 5

### Effect of NPTX1 Expression on Pluripotency Gene Expression and Role in the CRIPTO-Dependent Nodal/TGF Beta Signaling Pathway

In order to further characterize the role of NPTX1 in hESC differentiation, expression of pluripotency genes (genes expressed in undifferentiated stem cells) in hESCs in which NPTX1 expression was knocked down was determined.

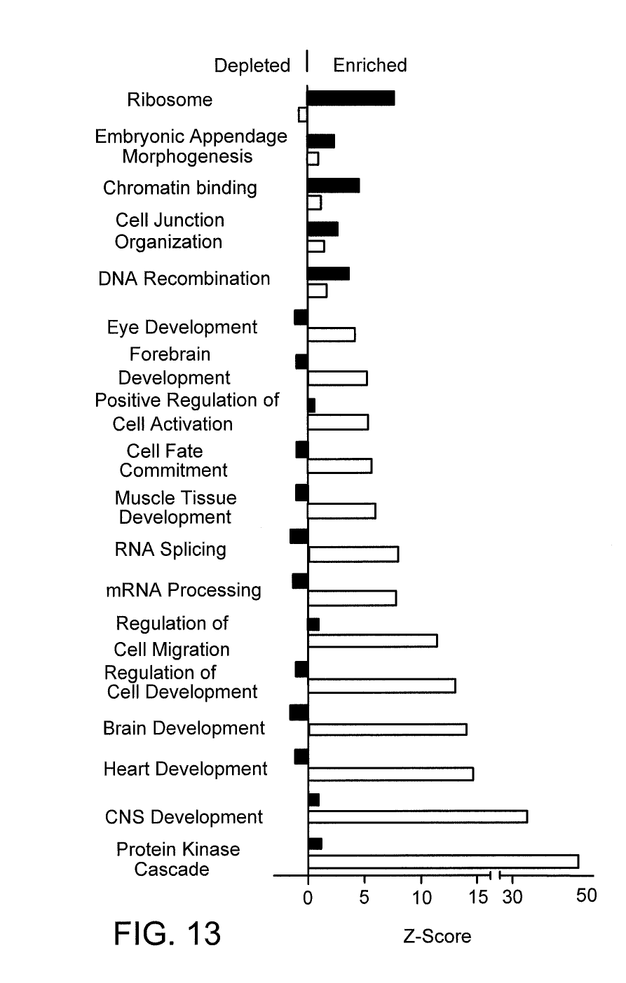

High-resolution temporal gene expression profiles were generated at five time points during the nine-day, dual SMAD inhibition protocol for neural differentiation of hESCs. Global gene expression studies were carried out in three independent samples for each time point and culture condition. The data was analyzed for genes with significant changes in their expression profiles by regressing the normalized expression values using polynomial least squares regression and performing an ANOVA on the coefficients of regression to identify genes with significant changes and at least a 2 fold difference between their high and low expression over the time course for each group. Notably, Gene Ontology categories of genes involved in development of various tissues and cell differentiation, including brain development and CNS categories, were greatly enriched in control cells, whereas these categories were either not significantly enriched or in some cases were depleted in cells treated with NPTX1 shRNA, indicating that NPTX1 shRNA reduced neural differentiation (FIG. 13). FIG. 13 shows the data for day 7 of the differentiation protocol.

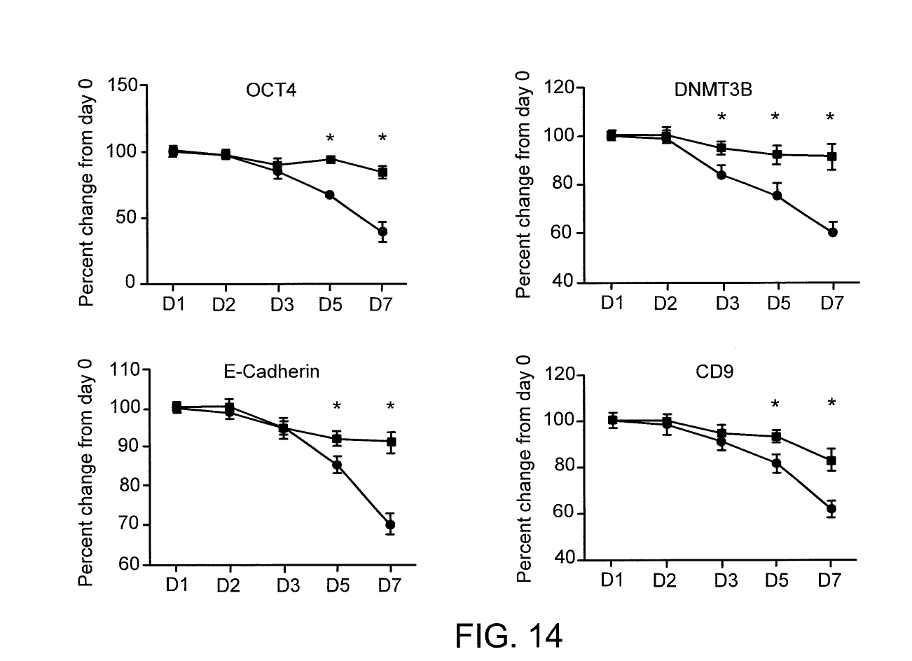

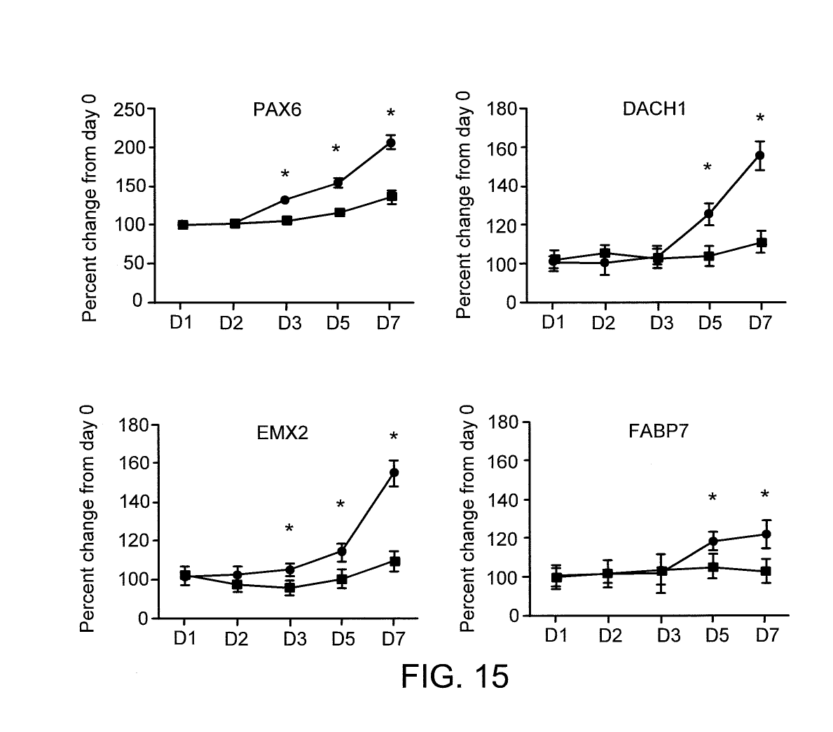

Furthermore, the NPTX1 shRNA expression profile showed enrichment for genes involved in pluripotency compared to the control expression profile. In particular, genes such as OCT-4, DNMT3B, E-Cadherin, and CD9 failed to reduce over time during neural differentiation, as they did in control hESCs. This was confirmed by qRT-PCR (FIG. 14). Among the transcripts highly decreased in NPTX1 shRNA-treated versus control cultures at day 7 of differentiation were genes associated with neural development such as PAX6, DACH1, EMX2, and FABP7 (FIG. 15). Together these data confirmed previous experiments showing NPTX1 shRNA reduced neural commitment from hESCs, and demonstrates that NPTX1 is a potential regulator of pluripotency.

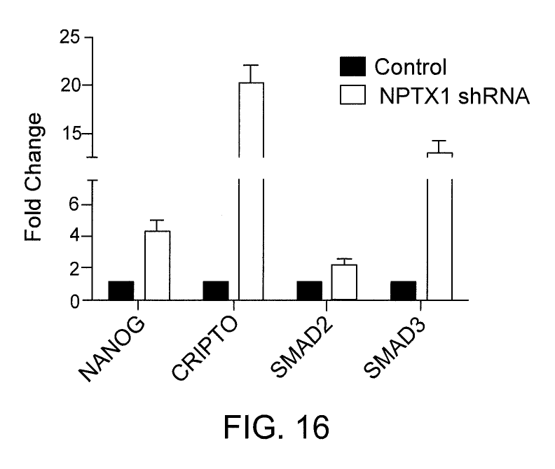

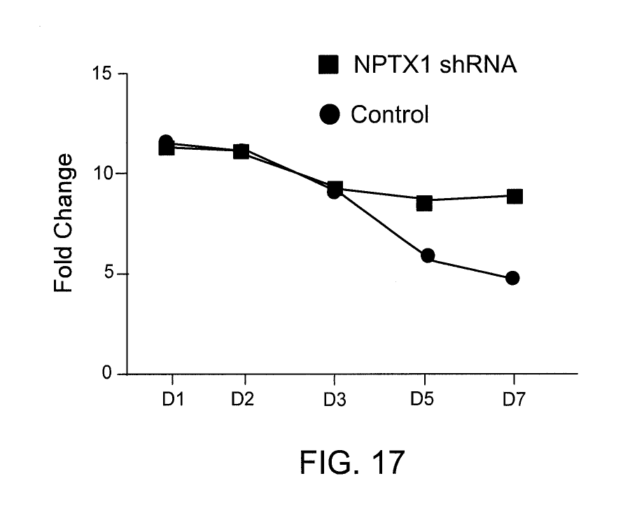

Moreover, in cells transduced with NPTX1 shRNA2, elevated levels of the undifferentiated stem cell genes, NANOG and OCT4, were observed at day 7 of differentiation compared to control (cells transduced with empty vector) (FIG. 16), indicating less differentiation. Additionally, there was a substantial elevation of CRIPTO, a gene expressed in hESCs. Along the neural differentiation time course, CRIPTO expression failed to completely drop off in cells treated with NPTX1 shRNA2 resulting in a 20-fold higher expression at day 7 of neural differentiation, compared to control-treated cells (FIG. 17). In cells transduced with NPTX1 shRNA2, elevated gene expression, determined by RT-PCR, of both SMAD2 and SMAD3, along with NANOG, was observed (FIG. 16).

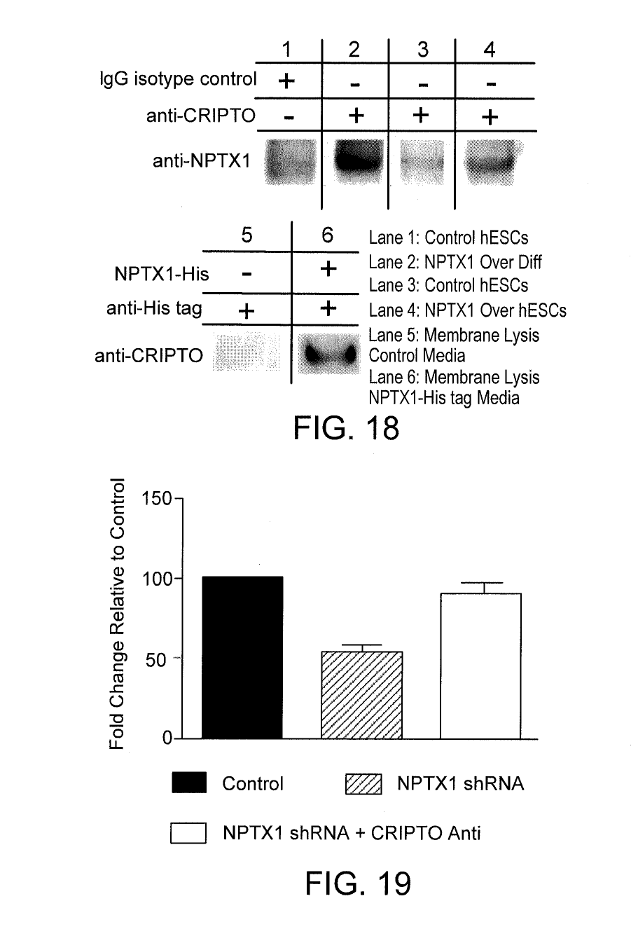

Finally, an NPTX1-His tagged vector was made by adding six HIS amino acids onto the N-terminus of the NPTX1 protein and used to make a conditioned media containing NPTX1-His. This media was applied to hESCs for 1 hour and a co-IP was performed on the membrane fraction of the lysed cells. CRIPTO was found to coimmunoprecipitate with the NPTX1-His protein (FIG. 18, lane 6), whereas CRIPTO did not coimmunoprecipitate in the membrane lysate from control treated cells (FIG. 18, lane 5).

Together, these data demonstrated that NPTX1 binds CRIPTO on the cell surface, as well as in the extracellular space, providing a mechanism for how NPTX1 can modulate Nodal signaling. NPTX1 could bind to CRIPTO, thereby lowering the availability of CRIPTO for the Nodal receptor, and in turn blocking the signaling mediated by CRIPTO that leads to neural induction. It was hypothesized that, if this indeed was the case, then blocking CRIPTO should rescue the deficit in neural induction brought about by NPTX1 shRNA. To test this hypothesis, a CRIPTO blocking antibody was used to block CRIPTO function in hESCs and in hESCs transduced with NPTX1 shRNA2 to knock down NPTX1 expression (“NPTX1 shRNA”). It was found that blocking CRIPTO in NPTX1 shRNA2-transduced cells allowed for neural induction to proceed in these NPTX1-deficient cells like the control (cells transduced with empty vector), as shown by the recovery of PAX6 gene expression in the cells treated with CRIPTO blocking antibody (FIG. 19). These data strongly suggested that NPTX1 induces differentiation of neural cells from the human embryo by binding CRIPTO, which in turn reduces the level of Nodal signaling, thereby allowing for pluripotency exit, and neural commitment.

### Example 6

### NPTX1 Regulates BMP Signaling via CRIPTO Regulation

Secreted proteins expressed during development have been shown to inhibit multiple signaling pathways. Cerberus is a secreted protein that has been shown to inhibit both the Nodal and BMP pathways. The directed neural differentiation paradigm used in this study employed the use of both a TGF-beta blocker (SB431542) and Noggin, a BMP blocker, as it has been shown that blocking only TGF-beta is not sufficient to drive neural commitment. While CRIPTO works to enhance NODAL signaling, it has been shown that NODAL itself can bind up BMPs and thus lower BMP signaling. Because NPTX1 depletes available CRIPTO in the extracelluar space and cell surface, it seemed plausible that the resulting excess Nodal could dampen BMP signaling. To test whether NPTX1 expression can reduce BMP signaling, the dual SMAD protocol was employed to generate neural cells (addition of Noggin and TGF-beta inhibitor SB431542).

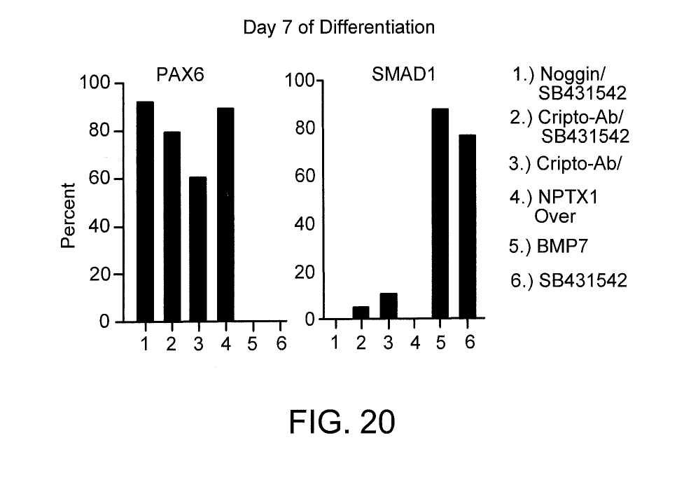

In one condition, Noggin was substituted with a CRIPTO blocking antibody to see if CRIPTO loss could result in robust neural differentiation. As a positive control for BMP signaling, another group was treated with high levels of BMP7. After 7 days of differentiation, neural marker expression and SMAD1 levels were determined. As expected, dual SMAD inhibition, via Noggin and SB431542, led to robust PAX6 expression and negligible levels of pSMAD1 (FIG. 20). hESCS treated with BMP7 or SB431542 (TGF-beta antagonist) alone gave rise to few PAX6 cells and exhibited high pSMAD1 levels (i.e., differentiated into neural cells) (FIG. 20). When SB431542 was combined with the CRIPTO blocking antibody, robust PAX6 expression was observed, along with low levels of SMAD1 (FIG. 20). Finally, the CRIPTO blocking antibody alone gave high PAX6 expression and lowered SMAD1 levels as well (FIG. 20). While this effect was quite robust, it did not yield PAX6 levels significantly similar to that of the dual SMAD inhibition (Noggin/SB431542) and NPTX1 over-expression. Together these data indicate that NPTX1 increases neural specification in part by reducing BMP signaling via CRIPTO regulation.

### Example 7

### Knockdown of NPTX1 in Induced Pluripotent Stem Cells

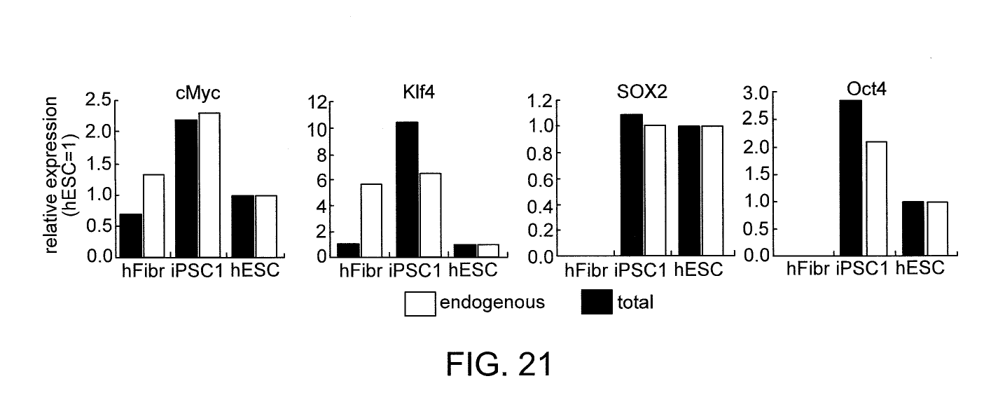

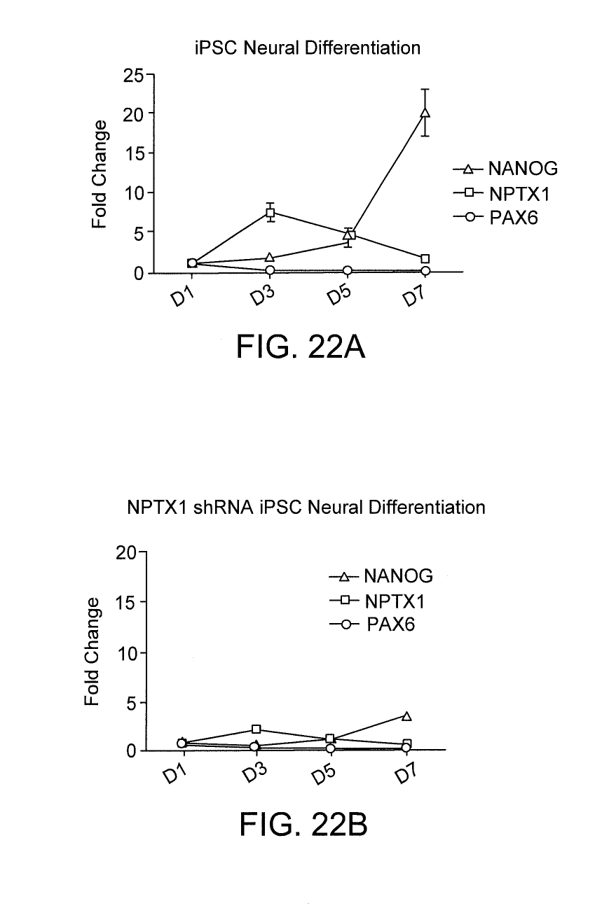

To verify NPTX1's role in other pluripotent cell lines, induced pluripotent stem cells (iPSCs) were generated, as describe above, and expression of pluripotency markers and viral integration were verified according to standard verification procedures (see, Maherali N, Hochedlinger K. Guidelines and techniques for the generation of induced pluripotent stem cells. Cell Stem Cell 2008; 3:595-605). The iPSCs expressed KLF4, c-Myc, Nanog, SSEA-4, Oct4, Sox2, Tra-1-60 and SSEA1, as determined by immunohistochemistry. Using qRT-PCR, levels of endogenous and total mRNA for cMyc, Klf4, SOX2 and Oct4 was quantified (FIG. 21). The total mRNA level included the endogenous and the exogenous gene expression, i.e., the exogenous transcripts made from the viral vector. The exogenous and endogeous mRNA was distinguished by using primer sequences that are specific to either endogenous or exogenous mRNA as described in Takahashi K, et al. PLoS One 2009;4:e8067. Subtraction between the two gave the endogenous expression. The iPSCs or iPSCs treated with NPTX1 shRNA to knock down NPTX1 expression were then differentiated toward the neural lineage using the dual SMAD inhibition protocol for neural differentiation. Expression levels of nanog, NPTX1 and PAX6 were determined by qRT-PCR. Similar to hESCs, a transient increase in NPTX1 expression preceding the increase of the neural marker PAX6 was observed in iPSCs (FIG. 22A). In NPTX1 shRNA treated iPSCs differentiated toward the neural lineage, PAX6 levels were reduced (FIG. 22B), which was consistent with results obtained for hESCs treated with NPTX1 shRNA (Example 2, supra).

### Example 8

### Role of Pentraxins in Neural Differentiation

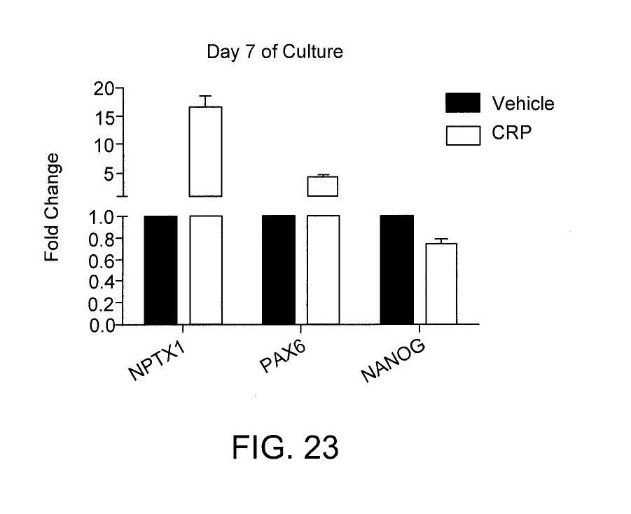

C-Reactive Protein (CRP) is a pentraxin protein that is found in the blood, and levels of which rise in response to stress and/or inflammation. Interestingly, it has been reported that serum CRP levels are higher in pregnant woman than in non-pregnant woman. It was also reported that, when CRP levels went over a particular threshold at 4-weeks of gestation, the time at which nervous system development occurs, an increased rate of miscarriage was identified. In the present Example, it was thus tested whether another member of the pentraxin protein family, in addition to NPTX1 might play an important role in neural differentiation. Recombinant CRP(R&D Systems, 2 μg/ml) or a vehicle control was added to hESCs cultured in conditions that would allow for neural development to proceed normally in the dish. After 7 days of CRP treatment, with CRP administered on days 0, 3, and 5, a striking up-regulation of neural marker PAX6 compared to the control was observed. Interestingly, this increase was accompanied by an increase in NPTX1 (FIG. 23). This data suggested that high levels of CRP may induce NPTX1 leading to pre-mature nervous system induction. Such acceleration of human development could be a cue for miscarriage and/or neural birth defects in fetuses exposed to high levels of CRP during this critical time of gestation. These data also suggested that other members of the pentraxin family of proteins can play an important and potentially overlapping role in directing neural differentiation.

### Discussion

The loss of function studies using NPTX1 shRNA (Example 2) demonstrated that hESCs are significantly impaired in generating neural cells using standard protocols in the absence of NPTX1, while the gain-in-function studies (NPTX1 overexpression) (Example 3) demonstrated that NPTX1 drives robust, spontaneous neural differentiation of hESCs. Importantly, CM from NPTX1-expressing fibroblasts can induce neural differentiation of hESCs (Example 4). These results are accompanied by a time course transcriptome analysis and functional assays (Example 5) revealing a role for NPTX1 in inhibiting the CRIPTO-dependent TGF-Beta/Nodal signaling pathway, a process previously described in mouse to be essential for neural induction. Further, it was demonstrated that NPTX1 regulates BMP signaling, another pathway critically involved in neural differentiation (Example 6). The critical role of NPTX1 in neural differentiation was not limited to hESCs, as similar findings were obtained in studies using iPSCs (Example 7). These studies thus reveal for the first time a transiently expressed protein (NPTX1) that, by itself, can initiate neural induction in hESCs and iPSCs. It is also demonstrated that hESCs cultured in the presence of recombinant CRP upregulate NPTX1 and PAX6, indicating that the ability to drive neural differentiation may involve other pentraxin proteins in addition to NPTX1 (Example 8). Thus, it is possible that CRP and other pentraxins could mimic the functions of NPTX1 demonstrated herein, and that NPTX1 may be used like CRP as a diagnostic tool during early pregnancy to monitor early nervous system developmental defects.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are incorporated by reference in their entirety as if physically present in this specification and to the same extent as if each item had been incorporated by reference individually. However, the citation of any such material, even in discussing the Background of this invention, is not to be construed as an admission that the material was or is available as prior art to the present invention.

What is claimed is:

1. A method for obtaining a neural cell, the method comprising administering to a stem or progenitor cell an inhibitor of the Nodal/TGF beta signaling pathway or an inhibitor of the BMP signaling pathway.

2. The method according to claim 1, wherein said inhibitor is a pentraxin selected from the group consisting of NPTX1, NPTX2 and CRP.

3. The method according to claim 2, wherein a pentraxin polypeptide is administered to the stem cell or progenitor cell.

4. The method according to claim 2, wherein an expression construct encoding the pentraxin polypeptide is administered to the stem cell or progenitor cell such that the stem cell or progenitor cell expresses the pentraxin polypeptide encoded by the expression construct.

5. The method according to claim 2, wherein said pentraxin is NPTX1.

6. The method according to claim 1, wherein the stem cell is selected from the group consisting of a skin stem cell, a spermatagonial stem cell, a hair follicle stem cell, a cancer stem cell, a bone marrow stem cell, a gut stem cell, a hematopoietic stem cell, an adipose stem cell, a mammalian embryonic stem cell (ESC), a retinal pigment epithelial stem cell, a mesenchymal stem cell, an epiblast stem cell, a renal stem cell, an amniotic stem cell, an umbilical blood stem cell, an endothelial stem cell, a neural crest stem cell, and an induced pluripotent stem cell (iPSC).

7. The method according to claim 1, wherein said inhibitor binds to CRIPTO.

8. The method according to claim 6, wherein said mammalian ESC is a human ESC.

9. A method for treating a disease or condition associated with aberrant Nodal/TGF beta signaling or aberrant BMP signaling, the method comprising administering to a subject in need thereof an effective amount for treating said condition of NPTX1.

10. The method according to claim 9, wherein NPTX1 is administered as a polypeptide or as a construct encoding NPTX1 polypeptide.

11. The method according to claim 9, wherein said disease or condition is a member selected from the group consisting of cancer, heart disease, muscular dystrophy, stroke, blood aneurisms and vessel aneurisms.

12. A pharmaceutical composition comprising NPTX1 and a pharmaceutically acceptable carrier.

13. A method for treating a disease or condition associated with aberrant Nodal/TGF beta signaling or aberrant BMP signaling, the method comprising administering to a subject in need thereof a therapeutically effective amount of the pharmaceutical composition of claim 9.

14. A method for maintaining a mammalian stem or progenitor cell in an undifferentiated state, the method comprising incubating the stem or progenitor cell in the presence of an inhibitor of NPTX1.

15. The method according to claim 14, wherein the stem cell is an embryonic stem cell (ESC).

16. The method according to claim 15, wherein the ESC is a human ESC.

17. A method for increasing differentiation of stem or progenitor cells toward a mesodermal lineage, the method comprising administering to the stem or progenitor cells an inhibitor of the Nodal/TGF beta signaling pathway or an inhibitor of the BMP signaling pathway, wherein the stem or progenitor cells are cultured in conditions appropriate for mesodermal differentiation.

18. The method according to claim 17, wherein the mesodermal lineage is selected from the group consisting of blood, heart, skeletal muscle, and smooth muscle.

19. The method according to claim 17, wherein said inhibitor is administered in an effective amount for increasing mesodermal differentiation such that the resulting population of cells consists of at least 60% cells of the mesodermal lineage.

20. The method according to claim 19, wherein said population of cells consists of at least 90% cells of the mesodermal lineage.
